(19) United States
(12) Patent Application Publication
(10) Pub. No.: US 2026/0317404 A1
(43) Pub. Date: Oct. 15, 2026

(54) B. BURGDORFERI PEPTIDOGLYCAN ANTIBODIES AND USES THEREOF

(71) Applicant: VIRGINIA TECH INTELLECTUAL PROPERTIES, INC., Blacksburg, VA (US)

(72) Inventor: Brandon Jutras, Blacksburg, VA (US)

(21) Appl. No.: 19/482,683

(22) PCT Filed: May 9, 2024
(86) PCT No.: PCT/US2024/028602

§ 371 Date: Nov. 7, 2025

Related U.S. Application Data
(60) Provisional application No. 63/465,024, filed on May 9, 2023.

(51) Int. Cl. C07K 16/1203; A61K 31/43; A61K 31/53; A61K 31/65; A61K 45/06; A61K 47/68; G01N 33/569

(52) U.S. Cl. CPC C07K 16/1207; A61K 31/43; A61K 31/53; A61K 31/65; A61K 45/06; A61K 47/6835; G01N 33/56911; C07K 2317/24; G01N 2333/20

(57) ABSTRACT

Described herein are compositions, assays, techniques, methods, for treating and/or detecting a Borrelia burgdorferi microorganism, the causative agent of Lyme disease.

### CROSS-REFERENCE TO RELATED APPLICATIONS

This application claims the benefit of and priority to U.S. Provisional Patent Application No. 63/465,024, filed on May 9, 2023, the contents of which is incorporated by reference herein in its entirety.

### SEQUENCE LISTING

This application contains a sequence listing filed in electronic form as an xml file entitled “VTIP-0430WP_ST26.xml”, created on May 8, 2024, and having a size of 32,266 bytes. The content of the sequence listing is incorporated herein in its entirety.

### TECHNICAL FIELD

The subject matter disclosed herein is generally directed to antibodies that target B. burgdorferi, components thereof, or produced therefrom.

### BACKGROUND

The pathogenic Borreliae spirochaetes are capable of causing significant disease in humans and animals. For example, Borrelia burgdorferi is estimated to cause more than 450,000 cases of Lyme disease each year in the U.S. alone. B. burgdorferi causes a bi-phasic infection with the acute stage being characterized by ‘flu-like’ symptoms, which is followed by a severe late-stage infection that can involve multiple organ systems and cause severe morbidity and can be fatal. Very little is known about what causes the clinical symptoms and there are few clinically relevant assays to test for infection, particularly active infection. As such there is a critical need for improved disease characterization and understanding and for methods of detecting B. burgdorferi for assessment and management of the infection and resulting Lyme disease.

Citation or identification of any document in this application is not an admission that such a document is available as prior art to the present invention.

### SUMMARY

In some aspects, the techniques described herein relate to an antibody or fragment thereof capable of specifically binding a Borreliae peptidoglycan.

In some aspects, the techniques described herein relate to an antibody or fragment thereof, wherein the Borreliae peptidoglycan is a Borrelia burgdorferi, Borrelia afzelii, Borrelia garinii, Borrelia japonica, Borrelia andersonii, Borrelia bissettii, Borrelia sinica, Borrelia turdi, Borrelia tanukii, Borrelia valaisiana, Borrelia lusitaniae, Borrelia spielmanii, Borrelia miyamotoi, or a Borrelia Lonestar peptidoglycan.

In some aspects, the techniques described herein relate to an antibody or fragment thereof, wherein the antibody or fragment thereof is capable of binding (a) a glycan component of the Borreliae peptidoglycan, optionally a GlcNAc-GlcNAc-anhMurNAc, (b) an alpha-Orn linkage in a Borreliae peptidoglycan, (c) a gamma-Orn linkage in a Borreliae peptidoglycan, or (d) a Borreliae muropeptide including AnuMurNAc.

In some aspects, the techniques described herein relate to an antibody or fragment thereof, wherein the antibody or fragment thereof includes one or more polypeptides each independently having a sequence that is 80-100 percent identical to one or more of SEQ ID NO: 1-8.

In some aspects, the techniques described herein relate to an antibody or fragment thereof, wherein the antibody is a chimeric antibody, a human antibody, or a humanized antibody.

In some aspects, the techniques described herein relate to an antibody or fragment thereof, wherein the antibody or fragment thereof includes a Fab, a Fab′, a F(ab′)2, a Fv, a single chain Fv (scFv), or combinations thereof.

In some aspects, the techniques described herein relate to an antibody or fragment thereof, wherein the antibody or fragment thereof is selected from the group consisting of: an IgG1, an IgG2, an IgG3, an IgG4, a variant thereof, and any combination thereof.

In some aspects, the techniques described herein relate to an antibody or fragment thereof, wherein the antibody or fragment thereof is linked to an agent, thereby forming an immunoconjugate.

In some aspects, the techniques described herein relate to an antibody or fragment thereof, wherein the antibody or fragment thereof is a monoclonal antibody or fragment thereof.

In some aspects, the techniques described herein relate to an antibody or fragment thereof, wherein the monoclonal antibody is selected from 1A4-1, 1E10, 1G11, or 2H10.

In some aspects, the techniques described herein relate to a polynucleotide encoding the antibody or fragment thereof of the present disclosure.

In some aspects, the techniques described herein relate to a polynucleotide encoding the antibody or fragment thereof of the present disclosure, wherein the polynucleotide includes or consists of a sequence that is 70-100 percent identical to any one of SEQ ID NOs: 9-16.

In some aspects, the techniques described herein relate to a vector including a polynucleotide encoding the antibody or fragment thereof of the present disclosure.

In some aspects, the techniques described herein relate to a cell or population thereof that includes (a) an antibody or fragment thereof of the present disclosure, (b) a polynucleotide encoding the antibody or fragment thereof of the present disclosure, (c) a vector of the present disclosure, or (d) any combination of (a)-(c)

In some aspects, the techniques described herein relate to a pharmaceutical formulation that includes (a) an antibody or fragment thereof of the present disclosure, (b) a polynucleotide encoding the antibody or fragment thereof of the present disclosure, (c) a vector of the present disclosure, (d) a cell or population thereof of the present disclosure, or (e) any combination of (a)-(d); and a pharmaceutically acceptable carrier. In some aspects, the techniques described herein relate to a pharmaceutical formulation, further including a co-therapeutic agent, wherein the co-therapeutic agent is effective to treat Lyme disease or a symptom thereof.

In some aspects, the techniques described herein relate to a kit including: (a) an antibody or fragment thereof of the present disclosure, (b) a polynucleotide encoding the antibody or fragment thereof of the present disclosure, (c) a vector of the present disclosure, (d) a cell or population thereof of the present disclosure, or (e) any combination of (a)-(d); (e) a pharmaceutical formulation of the present disclosure; or (f) any combination of (a)-(e).

In some aspects, the techniques described herein relate to a method of detecting and/or quantifying a Borreliae peptidoglycan, and/or diagnosing a Borreliae infection, and/or Lyme disease in a subject in need thereof, including the step of contacting the antibody or fragment thereof of the present disclosure with a biological sample from the subject in need thereof and detecting and/or quantifying the amount of Borreliae peptidoglycan in the sample.

In some aspects, the techniques described herein relate to a method wherein the Borreliae peptidoglycan is a Borrelia burgdorferi, Borrelia afzelii, Borrelia garinii, Borrelia japonica, Borrelia andersonii, Borrelia bissettii, Borrelia sinica, Borrelia turdi, Borrelia tanukii, Borrelia valaisiana, Borrelia lusitaniae, Borrelia spielmanii, Borrelia miyamotoi, or a Borrelia Lonestar peptidoglycan.

In some aspects, the techniques described herein relate to a method wherein the biological sample of the subject has a greater amount of Borreliae peptidoglycan as compared to a reference or control sample.

In some aspects, the techniques described herein relate to a method wherein the level of Borreliae peptidoglycan in the biological sample of the subject is greater than at least about 5%, at least about 10%, at least about 20%, at least about 30%, at least about 40%, at least about 50%, at least about 60%, at least about 70%, at least about 80%, at least about 90%, at least about 100%, at least about 150%, at least about 200%, at least about 300% or more compared to a corresponding level in the reference or control sample.

In some aspects, the techniques described herein relate to a method wherein the contacting and detecting and/or quantifying is performed in vitro.

In some aspects, the techniques described herein relate to a method wherein the biological sample comprises synovial fluid or serum.

In some aspects, the techniques described herein relate to a method that further comprises treating the subject with one or more therapeutic agents.

In some aspects, the techniques described herein relate to a method wherein the one or more therapeutic agents comprise (a) an antibody or fragment thereof of the present disclosure; (b) a polynucleotide of the present disclosure; (c) a vector of the present disclosure; (c) a cell or population thereof of the present disclosure; (d) a pharmaceutical formulation of the present disclosure; (e) a therapeutic agent effective to treat Lyme disease or a symptom thereof; or (f) any combination of (a)-(e).

In some aspects, the techniques described herein relate to a method wherein the one or more therapeutic agents comprise an antibody of the present disclosure, an antibiotic, a peptidoglycan-specific hydrolase, or any combination thereof. In some aspects, the techniques described herein relate to a method wherein the antibiotic comprises doxycycline, amoxicillin, ceftriaxone, or any combination thereof.

In some aspects, the techniques described herein relate to a method wherein treating occurs after detecting and/or diagnosing.

In some aspects, the techniques described herein relate to a method wherein the contacting is performed within about 3 weeks after suspected infection with a Borreliae organism.

In some aspects, the techniques described herein relate to a method the Borreliae organism is Borrelia burgdorferi, Borrelia afzelii, Borrelia garinii, Borrelia japonica, Borrelia andersonii, Borrelia bissettii, Borrelia sinica, Borrelia turdi, Borrelia tanukii, Borrelia valaisiana, Borrelia lusitaniae, Borrelia spielmanii, Borrelia miyamotoi, or Borrelia Lonestar.

In some aspects, the techniques described herein include a method of treating a Borreliae peptidoglycan disease or symptom thereof, a Borreliae infection or a symptom thereof, and/or Lyme disease or a symptom thereof in a subject in need thereof that includes administering (a) an antibody or fragment thereof of the present disclosure; (b) the polynucleotide of any one of the present disclosure; (c) the vector of the present disclosure; (c) the cell or population thereof of the present disclosure; (d) the pharmaceutical formulation of the present disclosure; or (e) any combination of (a)-(d) to the subject in need thereof.

In some aspects, the techniques described herein include a method of reducing, inhibiting, or ameliorating one or more symptoms of Lyme and/or Borrelia peptidoglycan disease in a subject in need thereof that includes administering to the subject an amount (a) an antibody or fragment thereof of the present disclosure; (b) the polynucleotide of the present disclosure; (c) the vector of the present disclosure; (c) the cell or population thereof of the present disclosure; (d) the pharmaceutical formulation of the present disclosure; or (e) any combination of (a)-(d).

In some aspects, the techniques described herein include a method wherein administering reduces the level of one or more pro-inflammatory mediators in the subject compared to a reference or a control.

In some aspects, the techniques described herein include a method wherein the one or more pro-inflammatory mediators comprise TNF-α, IL-1α, IL-1β, IL-6, IL-8, IFN-γ, IL-17F, or combinations thereof.

In some aspects, the techniques described herein include a method wherein the level of one or more pro-inflammatory mediators is reduced by at least about 5%, at least about 10%, at least about 20%, at least about 30%, at least about 40%, at least about 50%, at least about 60%, at least about 70%, at least about 80%, at least about 90% or more compared to the reference or the control.

In some aspects, the techniques described herein include a method wherein the subject is administered an additional therapeutic agent. In some aspects, the techniques described herein include a method wherein the additional therapeutic agent comprises an antibiotic or a peptidoglycan-specific hydrolase.

In some aspects, the techniques described herein include a method (a) wherein the antibiotic comprises doxycycline, amoxicillin, ceftriaxone, or combinations thereof, (b) wherein the peptidoglycan-specific hydrolase comprises an amidase or a lysozyme, or (c) both (a) and (b).

In some aspects, the techniques described herein include a method wherein the subject has or is suspected of having Lyme arthritis, neuroborreliosis or other Lyme disease-related clinical manifestations.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1A-1C shows a rabbit immunization strategy and response to Borrelia burgdorferi muropeptides. Two New Zeeland White Rabbits were immunized every 6 weeks with 5 doses of B. burgdorferi peptidoglycan that had been digested in the fragments (muropeptides). Both animals were tested for antibody titers against AnhMurNAc-L-Ala-D-Glu-alpha-L-Orn, (FIG. 1A) AnhMurNAc-L-Ala-D-Glu-gamma-L-Orn (FIG. 1B), or GlcNAc-GlcNAc-AnhMurNAc (FIG. 1C). Animal E19728 was used to collect splenocytes and test >500 B cell clones for specificity against each fragment above.

FIG. 2 can demonstrate refinement of B cell clones specific for muropeptides of interest and for counterselection. After screening >500 B cell clones, a subset was used for counter selection against the commonly released peptidoglycan fragments AnhMurNAc-L-Ala-D-Glu-L-Lys (gray) or AnhMurNAc-L-Ala-D-Glu-mDAP (black). 1A4, 1E10, 2G10, and 2H10 were selected for large-scale recombinant mAb production based on their specificity for target antigens of interest.

FIG. 3A-3B can demonstrate purification and analysis of select mAb on released peptidoglycan by live B. burgdorferi. (FIG. 3A) Purified mAb H and L chains were analyzed by SDS-PAGE. Lanes 1~4 correspond to mAbs 1A4, 1E10, 2G10, and 2H10, respectively. (FIG. 3B) ELISAs using recombinant mAb to detect released peptidoglycan present in culture supernatants following culture expansion (spent media) or intact peptidoglycan from cells (Intact PG). The results indicate that all four mAb are capable of detecting released peptidoglycan.

FIG. 4 shows a graph with results from immuno-PCR to detect fragments of Lyme disease causing Borreliae species peptidoglycan.

FIG. 5 shows a graph with results from an ELISA assay evaluating combinations of recombinant monoclonal antibodies for detecting fragments of released peptidoglycan in the serum of infected mice.

FIG. 6 shows a representative western blot demonstrating the efficacy of the anti-Borreliae peptidoglycan antibodies to detect Borreliae peptidoglycan in the urine of infected mice.

The figures herein are for illustrative purposes only and are not necessarily drawn to scale.

### DETAILED DESCRIPTION OF THE EXAMPLE EMBODIMENTS

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Where a range is expressed, a further aspect includes from the one particular value and/or to the other particular value. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure. For example, where the stated range includes one or both of the limits, ranges excluding either or both of those included limits are also included in the disclosure, e.g., the phrase “x to y” includes the range from ‘x’ to ‘y’ as well as the range greater than ‘x’ and less than ‘y’. The range can also be expressed as an upper limit, e.g., ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlett, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2nd edition (2011).

Definitions of common terms and techniques in chemistry and organic chemistry can be found in Smith. Organic Synthesis, published by Academic Press. 2016; Tinoco et al. Physical Chemistry, 5^{th} edition (2013) published by Pearson; Brown et al., Chemistry, The Central Science 14^{th} ed. (2017), published by Pearson, Clayden et al., Organic Chemistry, 2^{nd} ed. 2012, published by Oxford University Press; Carey and Sunberg, Advanced Organic Chemistry, Part A: Structure and Mechanisms, 5^{th} ed. 2008, published by Springer; Carey and Sunberg, Advanced Organic Chemistry, Part B: Reactions and Synthesis, 5^{th} ed. 2010, published by Springer, and Vollhardt and Schore, Organic Chemistry, Structure and Function; 8^{th} ed. (2018) published by W.H. Freeman.

Definitions of common terms, analysis, and techniques in genetics can be found in e.g., Hartl and Clark. Principles of Population Genetics. 4^{th} Ed. 2006, published by Oxford University Press. Published by Booker. Genetics: Analysis and Principles, 7^{th} Ed. 2021, published by McGraw Hill; Isik et la., Genetic Data Analysis for Plant and Animal Breeding. First ed. 2017. published by Springer International Publishing AG; Green, E. L. Genetics and Probability in Animal Breeding Experiments. 2014, published by Palgrave; Bourdon, R. M. Understanding Animal Breeding. 2000 2^{nd} Ed. published by Prentice Hall; Pal and Chakravarty. Genetics and Breeding for Disease Resistance of Livestock. First Ed. 2019, published by Academic Press; Fasso, D. Classification of Genetic Variance in Animals. First Ed. 2015, published by Callisto Reference; Megahed, M. Handbook of Animal Breeding and Genetics, 2013, published by Omniscriptum Gmbh & Co. Kg., LAP Lambert Academic Publishing; Reece. Analysis of Genes and Genomes. 2004, published by John Wiley & Sons. Inc; Deonier et al., Computational Genome Analysis. 5th Ed. 2005, published by Springer-Verlag, New York; Meneely, P. Genetic Analysis: Genes, Genomes, and Networks in Eukaryotes. 3rd Ed. 2020, published by Oxford University Press.

As used herein, the singular forms “a”, “an”, and “the” include both singular and plural referents unless the context clearly dictates otherwise.

As used herein, “about,” “approximately,” “substantially,” and the like, when used in connection with a measurable variable such as a parameter, an amount, a temporal duration, and the like, are meant to encompass variations of and from the specified value including those within experimental error (which can be determined by e.g. given data set, art accepted standard, and/or with e.g., a given confidence interval (e.g. 90%, 95%, or more confidence interval from the mean), such as variations of +/−10% or less, +/−5% or less, +/−1% or less, and +/−0.1% or less of and from the specified value, insofar such variations are appropriate to perform in the disclosed invention. As used herein, the terms “about,” “approximate,” “at or about,” and “substantially” can mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, “administering” refers to any suitable administration for the agent(s) being delivered and/or subject receiving said agent(s) and can be oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravireal, intracerebral, and intracerebroventricular, intratympanic, intracochlear, rectal, vaginal, by inhalation, by catheters, stents or via an implanted reservoir or other device that administers, either actively or passively (e.g. by diffusion) a composition the perivascular space and adventitia. For example, a medical device such as a stent can contain a composition or formulation disposed on its surface, which can then dissolve or be otherwise distributed to the surrounding tissue and cells. The term “parenteral” can include subcutaneous, intravenous, intramuscular, intra-articular, intra-synovial, intrasternal, intrathecal, intrahepatic, intralesional, and intracranial injections or infusion techniques. Administration routes can be, for instance, auricular (otic), buccal, conjunctival, cutaneous, dental, electro-osmosis, endocervical, endosinusial, endotracheal, enteral, epidural, extra-amniotic, extracorporeal, hemodialysis, infiltration, interstitial, intra-abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal (dental), intracoronary, intracorporus cavernosum, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratympanic, intrauterine, intravascular, intravenous, intravenous bolus, intravenous drip, intraventricular, intravesical, intravitreal, iontophoresis, irrigation, laryngeal, nasal, nasogastric, occlusive dressing technique, ophthalmic, oral, oropharyngeal, other, parenteral, percutaneous, periarticular, peridural, perineural, periodontal, rectal, respiratory (inhalation), retrobulbar, soft tissue, subarachnoid, subconjunctival, subcutaneous, sublingual, submucosal, topical, transdermal, transmucosal, transplacental, transtracheal, transtympanic, ureteral, urethral, and/or vaginal administration, and/or any combination of the above administration routes, which typically depends on the disease to be treated, subject being treated, and/or agent(s) being administered.

As used herein, a “biological sample” refers to a sample obtained from, made by, secreted by, excreted by, or otherwise containing part of or from a biologic entity. A biologic sample can contain whole cells and/or live cells and/or cell debris, and/or cell products, and/or virus particles. The biological sample can contain (or be derived from) a “bodily fluid”. The biological sample can be obtained from an environment (e.g., water source, soil, air, and the like). Such samples are also referred to herein as environmental samples. As used herein “bodily fluid” refers to any non-solid excretion, secretion, or other fluid present in an organism and includes, without limitation unless otherwise specified or is apparent from the description herein, amniotic fluid, aqueous humor, vitreous humor, bile, blood or component thereof (e.g. plasma, serum, etc.), breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from an organism, for example by puncture, or other collecting or sampling procedures.

As used herein, “fragment” as used throughout this specification with reference to a peptide, polypeptide, or protein generally denotes a portion of the peptide, polypeptide, or protein, such as typically an N- and/or C-terminally truncated form of the peptide, polypeptide, or protein. Preferably, a fragment may comprise at least about 30%, e.g., at least about 50% or at least about 70%, preferably at least about 80%, e.g., at least about 85%, more preferably at least about 90%, and yet more preferably at least about 95% or even about 99% of the amino acid sequence length of said peptide, polypeptide, or protein. For example, insofar not exceeding the length of the full-length peptide, polypeptide, or protein, a fragment may include a sequence of ≥5 consecutive amino acids, or ≥10 consecutive amino acids, or ≥20 consecutive amino acids, or ≥30 consecutive amino acids, e.g., ≥40 consecutive amino acids, such as for example ≥50 consecutive amino acids, e.g., ≥60, ≥70, ≥80, ≥90, ≥100, ≥200, ≥300, ≥400, ≥500 or ≥600 consecutive amino acids of the corresponding full-length peptide, polypeptide, or protein.

The term “optional” or “optionally” means herein that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

As used herein, “pharmaceutically acceptable carrier or excipient” refers to a carrier or excipient that is useful in preparing a pharmaceutical formulation that is generally safe, non-toxic, and is neither biologically or otherwise undesirable, and includes a carrier or excipient that is acceptable for veterinary use as well as human pharmaceutical use. A “pharmaceutically acceptable carrier or excipient” as used in the specification and claims includes both one and more than one such carrier or excipient.

As used herein, “pharmaceutical formulation” refers to the combination of an active agent, compound, or ingredient with a pharmaceutically acceptable carrier or excipient, making the composition suitable for diagnostic, therapeutic, or preventive use in vitro, in vivo, or ex vivo.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed. The subject can be any organism capable of being infected with or otherwise harboring (such as a vector) B. burgdorferi.

As used herein, the term “vector” or is used in reference to a vehicle used to introduce an exogenous nucleic acid sequence into a cell. A vector may include a DNA molecule, linear or circular (e.g., plasmids), which includes a segment encoding an RNA and/or polypeptide of interest operatively linked to additional segments that provide for its transcription and optional translation upon introduction into a host cell or host cell organelles. Such additional segments can include promoter and/or terminator sequences, and can also include one or more origins of replication, one or more selectable markers, an enhancer, a polyadenylation signal, etc. Expression vectors are generally derived from yeast or bacterial genomic or plasmid DNA, or viral DNA, or may contain elements of both. Expression vectors can be adapted for expression in prokaryotic or eukaryotic cells. Expression vectors can be adapted for expression in mammalian, fungal, yeast, or plant cells. Expression vectors can be adapted for expression in a specific cell type via the specific regulator or other additional segments that can provide for replication and expression of the vector within a particular cell type.

The term “Lyme disease” (also known as Lyme borreliosis) refers to a zoonotic, vector-borne disease caused by extracellular spirochetal bacteria from the genus Borrelia. Lyme disease is most often transmitted by the bite from an infected tick (e.g. the Ixodes tick). The infected nymphal tick transmits B. burgdorferi via its saliva to the human during its blood meal. Clinical presentation of Lyme disease is best known for the characteristic bull's-eye rash (also known as erythema chronicum migrans) but can also include myocarditis, cardiomyopathy, arrhythmia, arthritis, arthralgia, acrodermatitis, meningitis, neuropathies, and facial nerve palsy depending on the stage of infection. Lyme disease generally has three separate stages. Stage 1 is known as the Early Localized stage and occurs approximately 3 days-1 month after inoculation. Stage 1 is most commonly characterized by erythema migrans (a red “bull's-eye” rash seen as an erythematous circle encircling a defined center that expands outward). This skin lesion is often followed by flu-like symptoms, such as myalgia, arthralgia, headache and fever. If left untreated, the disease progresses to Stage 2 or the Early Disseminated stage, which generally occurs weeks-months after an infection. During Stage 2 the bacteria move into the blood stream from the site of infection and to more distant tissues and organs. Neurological, cardiovascular and arthritic symptoms that occur in this stage include meningitis, cranial neuropathy and intermittent inflammatory arthritis. Stage 3 is known as the Late Disseminated stage and occurs months-years after an initial infection. Symptoms can become chronic and can include encephalitis, meningitis, and arthritis (particularly of the knee).

The term “peptidoglycan” (also known as murein), a major component of the bacterial cell wall, refers to a polymer consisting of sugars and amino acids that forms a mesh-like layer that surrounds the cytoplasmic membrane of almost all bacteria (including Borreliae organisms such as Borrelia burgdorferi, Borrelia afzelii, Borrelia garinii, Borrelia japonica, Borrelia andersonii, Borrelia bissettii, Borrelia sinica, Borrelia turdi, Borrelia tanukii, Borrelia valaisiana, Borrelia lusitaniae, Borrelia spielmanii, Borrelia miyamotoi, or Borrelia Lonestar). The sugar component consists of alternating residues of β-(1,4) linked N-acetylglucosamine (NAG) and N-acetylmuramic acid (NAM). Attached to the N-acetylmuramic acid is a peptide chain of three to five amino acids. The peptide chain can be cross-linked to the peptide chain of another strand forming the 3D mesh-like layer. Peptidoglycan serves a structural role in the bacterial cell wall, giving structural strength, as well as counteracting the osmotic pressure of the cytoplasm. Peptidoglycan is also involved in binary fission during bacterial cell reproduction. In Gram-negative bacteria and Borreliae, the peptidoglycan is not surface-exposed, as it is encased by the outer membrane. (Rosa, P. A., et al., Nat Rev Microbiol 2005, 3(2): 129-43(2005); Höltje, J. V., Microbiol Mol Biol Rev 62(1): 181-203(1998)). To accommodate cell growth and incorporation of new peptidoglycan material, about 40% of the existing peptidoglycan is degraded at every generation (Park et al., Microbiol Mol Biol Rev 72(2): 211-227(2008)). Compared to most other bacteria, the peptidoglycan of B. burgdorferi, and other Borreliae species, has an unusual chemical composition. The peptidoglycan of most Gram-negative and Gram-positive bacteria have m-diaminopimelate and L-lysine, respectively, at the third amino acid position. In contrast, the peptidoglycan of B. burgdorferi has L-ornithine at the third amino acid position. This ornithine is linked to a glycine.

The term “recombinant host cell” (or simply “host cell”), as used herein, is intended to refer to a cell that comprises a nucleic acid that is not naturally present in the cell, and maybe a cell into which a recombinant expression vector has been introduced. It should be understood that such terms are intended to refer not only to the particular subject cell but to the progeny of such a cell. Because certain modifications can occur in succeeding generations due to either mutation or environmental influences, such progeny cannot, in fact, be identical to the parent cell, but are still included within the scope of the term “host cell” as used herein.

As used herein, the term “linked” refers to the association of two or more molecules. The linkage can be covalent or non-covalent. The linkage also can be genetic (i.e., recombinantly fused). Such linkages can be achieved using a wide variety of art recognized techniques, such as chemical conjugation and recombinant protein production.

As used herein, “administering” refers to the physical introduction of a therapeutic agent or a composition comprising a therapeutic agent to a subject, using any of the various methods and delivery systems known to those skilled in the art. Preferred routes of administration for antibodies described herein include intravenous, intraperitoneal, intramuscular, subcutaneous, spinal or other parenteral routes of administration, for example by injection or infusion. The phrase “parenteral administration” as used herein means modes of administration other than enteral and topical administration, usually by injection, and includes, without limitation, intravenous, intraperitoneal, intramuscular, intraarterial, intrathecal, intralymphatic, intralesional, intracapsular, intraorbital, intracardiac, intradermal, transtracheal, subcutaneous, subcuticular, intraarticular, subcapsular, subarachnoid, intraspinal, epidural and intrasternal injection and infusion, as well as in vivo electroporation. Alternatively, an antibody described herein can be administered via a non-parenteral route, such as a topical, epidermal or mucosal route of administration, for example, intranasally, orally, vaginally, rectally, sublingually or topically. Administering can also be performed, for example, once, a plurality of times, and/or over one or more extended periods.

The terms “treat,” “treating,” and “treatment,” as used herein, refer to any type of intervention or process performed on, or administering an active agent to, the subject with the objective of reversing, alleviating, ameliorating, inhibiting, or slowing down or preventing the progression, development, severity or recurrence of a symptom, complication, condition or biochemical indicia associated with a disease. Treatment can be of a subject having a disease or a subject who does not have a disease (e.g., for prophylaxis).

The term “diagnosis,” as used herein, refers to methods that can be used to determine or predict whether a patient is suffering from a given disease or condition (e.g., Lyme disease-related pathologies), thereby identifying a subject who is suitable for a treatment. A skilled artisan can make a diagnosis on the basis of one or more diagnostic marker (e.g., level of Borreliae species peptidoglycan (also referred to herein as a “Borreliae peptidoglycan”), (e.g., a Borrelia burgdorferi, Borrelia afzelii, Borrelia garinii, Borrelia japonica, Borrelia andersonii, Borrelia bissettii, Borrelia sinica, Borrelia turdi, Borrelia tanukii, Borrelia valaisiana, Borrelia lusitaniae, Borrelia spielmanii, Borrelia miyamotoi, and/or a Borrelia Lonestar peptidoglycan or level of antibodies against a Borreliae species peptidoglycan, (e.g., a Borrelia burgdorferi, Borrelia afzelii, Borrelia garinii, Borrelia japonica, Borrelia andersonii, Borrelia bissettii, Borrelia sinica, Borrelia turdi, Borrelia tanukii, Borrelia valaisiana, Borrelia lusitaniae, Borrelia spielmanii, Borrelia miyamotoi, and/or a Borrelia Lonestar peptidoglycan), where the presence, absence, amount, or change in amount of the diagnostic marker is indicative of the presence, severity, or absence of the condition. In some embodiments, an increase in the level of Borreliae peptidoglycan (e.g., a Borrelia burgdorferi, Borrelia afzelii, Borrelia garinii, Borrelia japonica, Borrelia andersonii, Borrelia bissettii, Borrelia sinica, Borrelia turdi, Borrelia tanukii, Borrelia valaisiana, Borrelia lusitaniae, Borrelia spielmanii, Borrelia miyamotoi, and/or a Borrelia Lonestar peptidoglycan) in a biological sample (e.g., synovial fluid) from a subject is indicative of Lyme disease or a Borreliae infection or a Borrelial disease. In some embodiments, an increase in the level of antibodies against Borreliae peptidoglycan (e.g., a Borrelia burgdorferi, Borrelia afzelii, Borrelia garinii, Borrelia japonica, Borrelia andersonii, Borrelia bissettii, Borrelia sinica, Borrelia turdi, Borrelia tanukii, Borrelia valaisiana, Borrelia lusitaniae, Borrelia spielmanii, Borrelia miyamotoi, and/or a Borrelia Lonestar peptidoglycan) in a biological sample (e.g., synovial fluid or serum) from a subject is indicative of Lyme disease or a Borreliae infection or a Borrelial disease. The term “diagnosis” does not refer to the ability to determine the presence or absence of a particular disease or disorder with 100% accuracy, or even that a given course or outcome is more likely to occur than not. Instead, the skilled artisan will understand that the term “diagnosis” refers to an increased probability that a certain disease or disorder is present in the subject. In some embodiments, the term “diagnosis” includes one or more diagnostic methods of identifying a subject who has Lyme disease.

The term “therapeutically effective amount” as used herein refers to an amount of a drug, alone or in combination with another therapeutic agent, effective to “treat” a disease or disorder in a subject or reduce the risk, potential, possibility or occurrence of a disease or disorder (e.g., Lyme disease and/or Lyme disease-related pathologies, e.g., arthritis, neurological disorder, and/or inflammatory condition). A “therapeutically effective amount” includes an amount of a drug or a therapeutic agent that provides some improvement or benefit to a subject having or at risk of having a disease or disorder (e.g., a Borreliae infection (i.e., an infection with a Borreliae species) or a Borrelial disease (i.e., a disease caused by a Borrelia infection), Lyme disease and/or Lyme disease-related pathologies, e.g., arthritis, neurological disorder, and/or inflammatory condition). Thus, a “therapeutically effective” amount is an amount that reduces the risk, potential, possibility or occurrence of a disease or provides disorder or some alleviation, mitigation, and/or reduces at least one indicator (e.g., arthritis), and/or decrease in at least one clinical symptom of a disease or disorder.

As used herein, “anti-infective” refers to compounds or molecules that can either kill an infectious agent and/or modulate or inhibit its activity, infectivity, replication, and/or spreading such that its infectivity is reduced or eliminated and/or the disease or symptom thereof that it is associated is less severe or eliminated. Anti-infectives include, but are not limited to, antibiotics, antibacterials, antifungals, antivirals, and antiprotozoals. Suitable anti-infectives include, but are not limited to, amebicides (e.g., nitazoxanide, paromomycin, metronidazole, tinidazole, chloroquine, miltefosine, amphotericin b, and iodoquinol), aminoglycosides (e.g., paromomycin, tobramycin, gentamicin, amikacin, kanamycin, and neomycin), anthelmintics (e.g., pyrantel, mebendazole, ivermectin, praziquantel, albendazole, thiabendazole, oxamniquine), antifungals (e.g., azole antifungals (e.g., itraconazole, fluconazole, posaconazole, ketoconazole, clotrimazole, miconazole, and voriconazole), echinocandins (e.g., caspofungin, anidulafungin, and micafungin), griseofulvin, terbinafine, flucytosine, and polyenes (e.g., nystatin, and amphotericin b), antimalarial agents (e.g., pyrimethamine/sulfadoxine, artemether/lumefantrine, atovaquone/proquanil, quinine, hydroxychloroquine, mefloquine, chloroquine, doxycycline, pyrimethamine, and halofantrine), antituberculosis agents (e.g., aminosalicylates (e.g., amino salicylic acid), isoniazid/rifampin, isoniazid/pyrazinamide/rifampin, bedaquiline, isoniazid, ethambutol, rifampin, rifabutin, rifapentine, capreomycin, and cycloserine), antivirals (e.g., amantadine, rimantadine, abacavir/lamivudine, emtricitabine/tenofovir, cobicistat/elvitegravir/emtricitabine/tenofovir, efavirenz/emtricitabine/tenofovir, abacavir/lamivudine/zidovudine, lamivudine/zidovudine, emtricitabine/tenofovir, emtricitabine/lopinavir/ritonavir/tenofovir, interferon alfa-2v/ribavirin, peginterferon alfa-2b, maraviroc, raltegravir, dolutegravir, enfuvirtide, foscarnet, fomivirsen, oseltamivir, zanamivir, nevirapine, efavirenz, etravirine, rilpivirine, delavirdine, nevirapine, entecavir, lamivudine, adefovir, sofosbuvir, didanosine, tenofovir, abacavir, zidovudine, stavudine, emtricitabine, zalcitabine, telbivudine, simeprevir, boceprevir, telaprevir, lopinavir/ritonavir, fosamprenavir, darunavir, ritonavir, tipranavir, atazanavir, nelfinavir, amprenavir, indinavir, saquinavir, ribavirin, valacyclovir, acyclovir, famciclovir, ganciclovir, and valganciclovir), carbapenems (e.g., doripenem, meropenem, ertapenem, and cilastatin/imipenem), cephalosporins (e.g., cefadroxil, cephradine, cefazolin, cephalexin, cefepime, ceflaroline, loracarbef, cefotetan, cefuroxime, cefprozil, loracarbef, cefoxitin, cefaclor, ceftibuten, ceftriaxone, cefotaxime, cefpodoxime, cefdinir, cefixime, cefditoren, cefizoxime, and ceftazidime), glycopeptide antibiotics (e.g., vancomycin, dalbavancin, oritavancin, and telavancin), glycylcyclines (e.g., tigecycline), leprostatics (e.g., clofazimine and thalidomide), lincomycin and derivatives thereof (e.g., clindamycin and lincomycin), macrolides and derivatives thereof (e.g., telithromycin, fidaxomicin, erythromycin, azithromycin, clarithromycin, dirithromycin, and troleandomycin), linezolid, sulfamethoxazole/trimethoprim, rifaximin, chloramphenicol, fosfomycin, metronidazole, aztreonam, bacitracin, penicillins (amoxicillin, ampicillin, bacampicillin, carbenicillin, piperacillin, ticarcillin, amoxicillin/clavulanate, ampicillin/sulbactam, piperacillin/tazobactam, clavulanate/ticarcillin, penicillin, procaine penicillin, oxacillin, dicloxacillin, and nafcillin), quinolones (e.g., lomefloxacin, norfloxacin, ofloxacin, moxifloxacin, ciprofloxacin, levofloxacin, Gemifloxacin, moxifloxacin, cinoxacin, nalidixic acid, enoxacin, grepafloxacin, gatifloxacin, trovafloxacin, and sparfloxacin), sulfonamides (e.g., sulfamethoxazole/trimethoprim, sulfasalazine, and sulfasoxazole), tetracyclines (e.g., doxycycline, demeclocycline, minocycline, doxycycline/salicylic acid, doxycycline/omega-3 polyunsaturated fatty acids, and tetracycline), and urinary anti-infectives (e.g., nitrofurantoin, methenamine, fosfomycin, cinoxacin, nalidixic acid, trimethoprim, and methylene blue).

The term “antibiotic” as used herein includes all commonly used bacteristatic and bactericidal antibiotics, usually those administered orally. Antibiotics include aminoglycosides, such as amikacin, gentamicin, kanamycin, neomycin, streptomycin, and tobramycin; cephalosporins, such as cefamandole, cefazolin, cephalexin, cephaloglycin, cephaloridine, cephalothin, cephapirin, and cephradine; macrolides, such as erythromycin and troleandomycin; penicillins, such as penicillin G, amoxicillin, ampicillin, carbenicillin, cloxacillin, dicloxacillin, methicillin, nafcillin, oxacillin, phenethicillin, and ticarcillin; polypeptide antibiotics, such as bacitracin, colistimethate, colistin, polymyxin B; tetracyclines, such as chlortetracycline, demeclocycline, doxycycline, methacycline, minocycline, tetracycline, and oxytetracycline; and miscellaneous antibiotics such as chloramphenicol, clindamycin, cycloserine, lincomycin, rifampin, spectinomycin, vancomycin, and viomycin. Additional antibiotics are described in “Remington's Pharmaceutical Sciences,” 16th Ed., (Mack Pub. Co., 1980), pp. 1121-1178.

As used herein with reference to the relationship between DNA, cDNA, CRNA, RNA, protein/peptides, and the like “corresponding to” or “encoding” (used interchangeably herein) refers to the underlying biological relationship between these different molecules. As such, one of skill in the art would understand that operatively “corresponding to” can direct them to determine the possible underlying and/or resulting sequences of other molecules given the sequence of any other molecule which has a similar biological relationship with these molecules. For example, from a DNA sequence an RNA sequence can be determined and from an RNA sequence a cDNA sequence can be determined.

As used herein, “suitable control” is a control that will be instantly appreciated by one of ordinary skill in the art as one that is included such that it can be determined if the variable being evaluated an effect, such as a desired effect or hypothesized effect. One of ordinary skill in the art will also instantly appreciate based on inter alia, the context, the variable(s), the desired or hypothesized effect, what is a suitable or an appropriate control needed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### Overview

The pathogenic spirochaete Borrelia burgdorferi is estimated to cause more than 450,000 cases of Lyme disease each year in the U.S. alone. B. burgdorferi causes a bi-phasic infection with the acute stage being characterized by ‘flu-like’ symptoms, which is followed by a severe late-stage infection that can involve multiple organ systems and cause severe morbidity and can be fatal. Very little is known about what causes the clinical symptoms and there are few clinically relevant assays to test for infection, particularly active infection. Other Borreliae species, such as Borrelia afzelii (B. afzelii) and Borrelia garinii (B. garinii) can also lead to Lyme disease. As such there is a critical need for improved disease characterization and understanding and for methods of detecting B. burgdorferi for assessment and management of the infection and resulting Lyme disease.

B. burgdorferi has similar structural features to other Borreliae in that it has a flexible cell wall composed of outer and inner/cytoplasmic membranes surrounding periplasmic flagella (Rosa, P. A., et al., Nat Rev Microbiol 3(2): 129-43 (2005)). The inner and outer membranes enclosed a thin layer of peptidoglycan. The outer membrane of B. burgdorferi is easily disrupted and does not contain lipopolysaccharide (LPS). A number of antigenic B. burgdorferi cell surface proteins have been identified. These include the outer membrane surface proteins (Osp) OspA, OspB, OspC and VIsE. OspA and OspB are encoded by tightly linked tandem genes which are transcribed as a single transcriptional unit (Brusca, J. et al., J. Bacteriol. 173:8004-8008 (1991)). The most-studied B. burgdorferi membrane protein is OspA, a lipoprotein antigen expressed by Borreliae in testing ticks and the most abundant protein expressed in vitro by most borrelial isolates (Barbour, A. G., et al., Infection & Immunity 41:795-804 (1983); Howe, T. R., et al., Science 227:645 (1985)).

Compared to most other bacteria, the peptidoglycan of B. burgdorferi has an unusual chemical composition. The peptidoglycan of most Gram-negative and Gram-positive bacteria have m-diaminopimelate and L-lysine, respectively, at the third amino acid position. In contrast, the peptidoglycan of B. burgdorferi has L-ornithine at the third amino acid position. This ornithine is linked to a glycine.

During infection, the B. burgdorferi sheds muropeptides into its extracellular environment. Without being bound by theory, the B. burgdorferi peptidoglycan can stimulate an immune response in the subject and is believed to play a role in disease progression, symptoms, and secondary conditions.

With that said, embodiments disclosed herein can provide an antibody or fragment thereof capable of specifically binding a B. burgdorferi peptidoglycan. Further embodiments can also provide polynucleotides encoding the antibody or fragment thereof, vectors, cells, pharmaceutical formulations, kits, and methods of detecting and/or diagnosing B. burgdorferi peptidoglycan, B. burgdorferi infection and/or Lyme disease. As supported by the Working Examples herein, these compositions and methods can extend to include other species of Borreliae that cause Lyme disease, such as Borrelia afzelii and Borrelia garinii. Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

### Antibodies and Fragments Thereof

Described in several exemplary embodiments herein are antibodies or fragments thereof capable of specifically binding a Borrelia burgdorferi (B. Burgdorferi) peptidoglycan. In certain example embodiments, the B. burgdorferi anti-peptidoglycan antibody or fragment thereof is capable of binding (a) a glycan component of the B. burgdorferi peptidoglycan, optionally a GlcNAc-GlcNAc-anhMurNAc, (b) an alpha-Orn linkage in a B. burgdorferi peptidoglycan, (c) a gamma-Orn linkage in a B. burgdorferi peptidoglycan, or (d) a B. burgdorferi muropeptide comprising AnhMurNAc. In some embodiments, the B. burgdorferi anti-peptidoglycan antibody or fragment thereof is capable of specifically binding (a) a glycan component of the B. burgdorferi peptidoglycan, optionally a GlcNAc-GlcNAc-anhMurNAc, (b) an alpha-Orn linkage in a B. burgdorferi peptidoglycan, (c) a gamma-Orn linkage in a B. burgdorferi peptidoglycan, or (d) a B. burgdorferi muropeptide comprising AnuMurNAc.

Described in several exemplary antibodies herein are antibodies or fragments thereof composed of or including one or more polypeptides each independently having a sequence that is 80%-100% identical to any one of SEQ ID NO: 1-8, such as 80%, to/or 81%, 82%, 83%, 84%, 85%, 86%, 87%, 88%, 89%, 90%, 91%, 92%, 93%, 94%, 95%, 96%, 97%, 98%, 99%, 100% identical to any one of SEQ ID NO: 1-8. In some embodiments, the antibody is a monoclonal antibody. In some embodiments the antibody is a polyclonal antibody. Methods of making antibodies, including polyclonal and monoclonal antibodies are described elsewhere herein and are generally known in the art. Without being bound by theory, the antibodies and fragments thereof described herein can be useful in methods of detecting and/or treating a Borreliae infection and/or a Borrelial disease, Lyme disease and/or Lyme disease-related pathologies as described in greater detail elsewhere herein. In certain example embodiments, the antibody or fragment thereof described herein comprises or is one or more of SEQ ID NO: 1-8. In certain example embodiments, the antibody or fragment thereof described herein comprises a chimeric antibody, a human antibody, or a humanized antibody. In certain example embodiments, the antibody comprises a monoclonal antibody or fragment thereof.

In some embodiments, the antibody or fragment thereof of the present disclosure binds preferentially to a Borreliae peptidoglycan over other types of bacterial peptidoglycans. In some embodiments, the antibody or fragment thereof of the present disclosure preferentially binds to a Borrelia burgdorferi, Borrelia afzelii, Borrelia garinii, Borrelia japonica, Borrelia andersonii, Borrelia bissettii, Borrelia sinica, Borrelia turdi, Borrelia tanukii, Borrelia valaisiana, Borrelia lusitaniae, Borrelia spielmanii, Borrelia miyamotoi, a Borrelia Lonestar peptidoglycan or any combination thereof over other types of bacterial peptidoglycans. In certain embodiments, the other type of bacterial peptidoglycan comprises peptidoglycan belonging to Escherichia coli, Bacillus subtilis, or Staphylococcus aureus. In some embodiments, the binding of the antibody or fragment thereof of the present disclosure to a Borreliae peptidoglycan reduces the pro-inflammatory activity of the peptidoglycan. In some embodiments, the Borreliae peptidoglycan is a Borrelia burgdorferi, Borrelia afzelii, Borrelia garinii, Borrelia japonica, Borrelia andersonii, Borrelia bissettii, Borrelia sinica, Borrelia turdi, Borrelia tanukii, Borrelia valaisiana, Borrelia lusitaniae, Borrelia spielmanii, Borrelia miyamotoi, a Borrelia Lonestar peptidoglycan or any combination thereof.

In certain example embodiments, the antibody or fragment thereof described herein comprises a Fab, a Fab′, a F(ab′)2, a Fv, a single chain Fv (scFv), or combination thereof. In certain example embodiments, the antibody or fragment thereof described herein is selected from the group consisting of an IgG1, an IgG2, an IgG3, an IgG4, a variant thereof, and any combination thereof. In certain example embodiments, the antibody or fragment thereof described herein is linked to an agent, thereby forming an immunoconjugate. In certain example embodiments, the antibody or fragment thereof is a monoclonal antibody, optionally selected from 1A4-1, 1E10, 1G11, or 2H10. Without being bound by theory, the antibodies and fragments thereof described herein can be useful in methods of detecting and/or treating a Borreliae infection and/or a Borrelial disease, Lyme disease and/or Lyme disease-related pathologiesas described elsewhere herein.

In some embodiments, an anti-Borreliae peptidoglycan antibody or fragment thereof and formulations thereof disclosed herein are effective to protect against other species of Borrelia including, but not limited to, Borrelia japonica, Borrelia andersonii, Borrelia bissettii, Borrelia sinica, Borrelia turdi, Borrelia tanukii, Borrelia valaisiana, Borrelia lusitaniae, Borrelia spielmanii, Borrelia miyamotoi, Borrelia garinii, Borrelia afzelii, or Borrelia lonestar. Accordingly, in some embodiments, an antibody or fragment thereof of the present disclosure can cross-react with the peptidoglycan of multiple species of Borreliae, including variants thereof. It will be appreciated that the cross-reaction can occur via binding a peptidoglycan produced, excreted, or otherwise associated with Borrelia japonica, Borrelia andersonii, Borrelia bissettii, Borrelia sinica, Borrelia turdi, Borrelia tanukii, Borrelia valaisiana, Borrelia lusitaniae, Borrelia spielmanii, Borrelia miyamotoi, Borrelia garinii, Borrelia afzelii, or Borrelia lonestar. Cross-reactivity can be determined by any suitable method, including but not limited to immuno-PCR, which is shown at least in the Working Examples herein. The antibodies or fragments thereof of the present disclosure does not cross react with peptidoglycan of non-Borreliae bacteria. In other words, the antibodies or fragments thereof of the present disclosure specifically bind only peptidoglycan from Borreliae.

The terms “antibody” and “antibodies” are terms of art and can be used interchangeably herein and refer to a molecule with an antigen binding site that specifically binds an antigen. The terms as used to herein include whole (full-length) antibodies and any antigen binding fragments (i.e., “antigen-binding portions”) or single chains thereof. An “antibody” refers, in one embodiment, to a glycoprotein comprising at least two heavy (H) chains and two light (L) chains inter-connected by disulfide bonds, or an antigen binding portion thereof. In another embodiment, an “antibody” refers to a single chain antibody comprising a single variable domain, e.g., VHH domain. Each heavy chain is comprised of a heavy chain variable region (abbreviated herein as VH) and a heavy chain constant region. In certain naturally occurring antibodies, the heavy chain constant region is comprised of three domains, CH1, CH2 and CH3. In certain naturally occurring antibodies, each light chain is comprised of a light chain variable region (abbreviated herein as VL) and a light chain constant region. The light chain constant region is comprised of one domain, CL.

The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (C1q) of the classical complement system.

Antibodies can be of any type (e.g., IgG, IgE, IgM, IgD, IgA or IgY), any class (e.g., IgD, IgG2, IgG3, IgG4, IgAQ1 or IgA2), or any subclass (e.g., IgG1, IgG2, IgG3 and IgG4 in humans, and IgG1, IgG2a, IgG2b and IgG3 in mice) of immunoglobulin molecule. Immunoglobulins, e.g., IgG1, exist in several allotypes, which differ from each other in at most a few amino acids. An antibody disclosed herein can be from any of the commonly known isotypes, classes, subclasses, or allotypes. In certain embodiments, the antibodies described herein are of the IgG1, IgG2, IgG3 or IgG4 subclass or any hybrid thereof. In certain embodiments, the antibodies are of the IgG2, IgG4 or IgG2/IgG4 subclass.

“Antibody” includes, by way of example, both naturally occurring and non-naturally occurring antibodies; monoclonal and polyclonal antibodies; chimeric and humanized antibodies; human and nonhuman antibodies; wholly synthetic antibodies; single chain antibodies; monospecific antibodies; multispecific antibodies (including bispecific antibodies); tetrameric antibodies comprising two heavy chain and two light chain molecules; an antibody light chain monomer; an antibody heavy chain monomer; an antibody light chain dimer, an antibody heavy chain dimer; an antibody light chain-antibody heavy chain pair; intrabodies; heteroconjugate antibodies; monovalent antibodies; single chain antibodies; camelized antibodies; affibodies; anti-idiotypic (anti-Id) antibodies (including, e.g., anti-anti-Id antibodies), and single-domain antibodies (sdAbs), which include binding molecules consisting of a single monomeric variable antibody domain that are fully capable of antigen binding (e.g., a VH domain or a VL domain). Harmen M. M. and Haard H. J. Appl Microbiol Biotechnol. 77(1): 13-22 (2007)).

The term “antigen-binding portion” of an antibody, as used herein, refers to one or more fragments of an antibody that retain the ability to specifically bind to an antigen (e.g., B. burgdorferi peptidoglycan). Such “fragments” are, for example between about 8 and about 1500 amino acids in length, suitably between about 8 and about 745 amino acids in length, suitably about 8 to about 300, for example about 8 to about 200 amino acids, or about 10 to about 50 or 100 amino acids in length. It has been shown that the antigen-binding function of an antibody can be performed by fragments of a full-length antibody. Examples of binding fragments encompassed within the term “antigen-binding portion” of an antibody, e.g., an anti-peptidoglycan antibody described herein, include (i) a Fab fragment, a monovalent fragment consisting of the VL, VH, CL, and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, and disulfide-linked Fvs (sdFv) (v) a dAb fragment (Ward et al., (1989) Nature 341:544-546), which consists of a VH domain; and (vi) an isolated complementarity determining region (CDR) or (vii) a combination of two or more isolated CDRs which can optionally be joined by a synthetic linker. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see, e.g., Bird et al., (1988) Science 242:423-426; and Huston et al., (1988) Proc. Natl. Acad. Sci. USA 85:5879-5883). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies. Antigen-binding portions can be produced by recombinant DNA techniques, or by enzymatic or chemical cleavage of intact immunoglobulins.

As used herein, the terms “variable region” or “variable domain” are used interchangeably and are common in the art. The variable region typically refers to a portion of an antibody, generally, a portion of a light or heavy chain, typically about the amino-terminal 110 to 120 amino acids in the mature heavy chain and about 90 to 115 amino acids in the mature light chain, which differ extensively in sequence among antibodies and are used in the binding and specificity of a particular antibody for its particular antigen. The variability in sequence is concentrated in those regions called complementarity determining regions (CDRs) while the more highly conserved regions in the variable domain are called framework regions (FR).

Without wishing to be bound by any particular mechanism or theory, it is believed that the CDRs of the light and heavy chains are primarily responsible for the interaction and specificity of the antibody with antigen. In certain embodiments, the variable region is a human variable region. In certain embodiments, the variable region comprises rodent or murine CDRs and human framework regions (FRs). In particular embodiments, the variable region is a primate (e.g., non-human primate) variable region. In certain embodiments, the variable region comprises rodent or murine CDRs and primate (e.g., non-human primate) framework regions (FRs).

As used herein, the term “heavy chain” when used in reference to an antibody can refer to any distinct type, e.g., alpha (α), delta (δ), epsilon (ε), gamma (γ) and mu (μ), based on the amino acid sequence of the constant domain, which give rise to IgA, IgD, IgE, IgG and IgM classes of antibodies, respectively, including subclasses of IgG, e.g., IgG1, IgG2, IgG3 and IgG4.

As used herein, the term “light chain” when used in reference to an antibody can refer to any distinct type, e.g., kappa (κ) or lambda (λ) based on the amino acid sequence of the constant domains. Light chain amino acid sequences are well known in the art. In specific embodiments, the light chain is a human light chain.

The terms “VL” and “VL domain” are used interchangeably to refer to the light chain variable region of an antibody.

The terms “VH” and “VH domain” are used interchangeably to refer to the heavy chain variable region of an antibody.

As used herein, the term “constant region” or “constant domain” are interchangeable and have its meaning common in the art. The constant region is an antibody portion, e.g., a carboxyl terminal portion of a light and/or heavy chain which is not directly involved in binding of an antibody to antigen but which can exhibit various effector functions, such as interaction with the Fc receptor. The constant region of an immunoglobulin molecule generally has a more conserved amino acid sequence relative to an immunoglobulin variable domain.

An “Fc region” (fragment crystallizable region) or “Fc domain” or “Fc” refers to the C-terminal region of the heavy chain of an antibody that mediates the binding of the immunoglobulin to host tissues or factors, including binding to Fc receptors located on various cells of the immune system (e.g., effector cells) or to the first component (C1q) of the classical complement system. Thus, an Fc region comprises the constant region of an antibody excluding the first constant region immunoglobulin domain (e.g., CH1 or CL). In IgG, IgA and IgD antibody isotypes, the Fc region comprises two identical protein fragments, derived from the second (CH2) and third (CH3) constant domains of the antibody's two heavy chains; IgM and IgE Fc regions comprise three heavy chain constant domains (CH domains 2-4) in each polypeptide chain. For IgG, the Fc region comprises immunoglobulin domains Cγ2 and Cγ3 and the hinge between Cγ1 and Cγ2. Although the boundaries of the Fc region of an immunoglobulin heavy chain might vary, the human IgG heavy chain Fc region is usually defined to stretch from an amino acid residue at position C226 or P230 (or amino acid between these two amino acids) to the carboxy-terminus of the heavy chain, wherein the numbering is according to the EU index as in Kabat. The CH2 domain of a human IgG Fc region extends from about amino acid 231 to about amino acid 340, whereas the CH3 domain is positioned on C-terminal side of a Cm domain in an Fc region, i.e., it extends from about amino acid 341 to about amino acid 447 of an IgG. As used herein, the Fc region can be a native sequence Fc, including any allotypic variant, or a variant Fc (e.g., a non-naturally occurring Fc). Fc can also refer to this region in isolation or in the context of an Fc-comprising protein polypeptide such as a “binding protein comprising an Fc region,” also referred to as an “Fc fusion protein” (e.g., an antibody or immunoadhesin).

A “native sequence Fc region” or “native sequence Fc” comprises an amino acid sequence that is identical to the amino acid sequence of an Fc region found in nature. Native sequence human Fc regions include a native sequence human IgG1 Fc region; native sequence human IgG2 Fc region; native sequence human IgG3 Fc region; and native sequence human IgG4 Fc region as well as naturally occurring variants thereof. Native sequence Fc includes the various allotypes of Fes (see, e.g., Jefferis et al., (2009) mAbs 1:1; Vidarsson G. et al., Front Immunol. 5:520 (published online Oct. 20, 2014)).

An “Fc receptor” or “FcR” is a receptor that binds to the Fc region of an immunoglobulin. FcRs that bind to an IgG antibody comprise receptors of the FcγR family, including allelic variants and alternatively spliced forms of these receptors. The FcγR family consists of three activating (FcγRI, FcγRIII, and FcγRIV in mice; FcγRIA, FcγRIIA, and FcγRIIIA in humans) and one inhibitory (FcγRIIB) receptor. Human IgG1 binds to most human Fc receptors and elicits the strongest Fc effector functions. It is considered equivalent to murine IgG2a with respect to the types of activating Fc receptors that it binds to. Conversely, human IgG4 elicits the least Fc effector functions. Vidarsson G. et al., Front Immunol. 5:520 (published online Oct. 20, 2014).

The constant region can be manipulated, e.g., by recombinant technology, to eliminate one or more effector functions. An “effector function” refers to the interaction of an antibody Fc region with an Fc receptor or ligand, or a biochemical event that results therefrom. Exemplary “effector functions” include C1q binding, complement dependent cytotoxicity (CDC), Fc receptor binding, FcγR-mediated effector functions such as ADCC and antibody dependent cell-mediated phagocytosis (ADCP), and down regulation of a cell surface receptor (e.g., the B cell receptor; BCR). Such effector functions generally require the Fc region to be combined with a binding domain (e.g., an antibody variable domain). Accordingly, the term “a constant region without the Fc function” include constant regions with reduced or without one or more effector functions mediated by Fc region.

Effector functions of an antibody can be reduced or avoided by different approaches. Effector functions of an antibody can be reduced or avoided by using antibody fragments lacking the Fc region (e.g., such as a Fab, F(ab′)₂, single chain Fv (scFv), or a sdAb consisting of a monomeric VH or VL domain). Alternatively, the so-called aglycosylated antibodies can be generated by removing sugars that are linked to particular residues in the Fc region to reduce the effector functions of an antibody while retaining other valuable attributes of the Fc region (e.g., prolonged half-life and heterodimerization). Aglycosylated antibodies can be generated by, for example, deleting or altering the residue the sugar is attached to, removing the sugars enzymatically, producing the antibody in cells cultured in the presence of a glycosylation inhibitor, or by expressing the antibody in cells unable to glycosylate proteins (e.g., bacterial host cells). See, e.g., U.S. Pub. No. 20120100140. Another approach is to employ Fc regions from an IgG subclass that have reduced effector function, for example, IgG2 and IgG4 antibodies are characterized by having lower levels of Fc effector functions than IgG1 and IgG3. The residues most proximal to the hinge region in the CH2 domain of the Fc part are responsible for effector functions of antibodies as it contains a largely overlapping binding site for C1q (complement) and IgG-Fc receptors (FcγR) on effector cells of the innate immune system. Vidarsson G. et al., Front Immunol. 5:520 (published online Oct. 20, 2014). Accordingly, antibodies with reduced or without Fc effector functions can be prepared by generating, e.g., a chimeric Fc region which comprises a CH2 domain from an IgG antibody of the IgG4 isotype and a CH3 domain from an IgG antibody of the IgG1 isotype, or a chimeric Fc region which comprises hinge region from IgG2 and CH2 region from IgG4 (see, e.g., Lau C. et al., J. Immunol. 191:4769-4777 (2013)), or an Fc region with mutations that result in altered Fc effector functions, e.g., reduced or no Fc functions. Such Fc regions with mutations are known in the art. See, e.g., U.S. Pub. No. 20120100140 and U.S. and PCT applications cited therein and An et al., mAbs 1:6, 572-579 (2009); the disclosure of which are incorporated by reference to their entirety.

A “hinge”, “hinge domain” or “hinge region” or “antibody hinge region” refers to the domain of a heavy chain constant region that joins the CH1 domain to the CH2 domain and includes the upper, middle, and lower portions of the hinge (Roux et al., J. Immunol. 1998 161:4083). The hinge provides varying levels of flexibility between the binding and effector regions of an antibody and also provides sites for intermolecular disulfide bonding between the two heavy chain constant regions. As used herein, a hinge starts at Glu216 and ends at Gly237 for all IgG isotypes (Roux et al., 1998 J Immunol 161:4083). The sequences of wild-type IgG1, IgG2, IgG3 and IgG4 hinges known in the art. See, e.g., Kabat E A et al., (1991) Sequences of Proteins of Immunological Interest, Fifth Edition, U.S. Department of Health and Human Services, NIH Publication No. 91-3242; Vidarsson G. et al., Front Immunol. 5:520 (published online Oct. 20, 2014).

The term “CH1 domain” refers to the heavy chain constant region linking the variable domain to the hinge in a heavy chain constant domain. As used herein, a CH1 domain starts at A118 and ends at V215. The term “CH1 domain” includes wildtype CH1 domains, as well as naturally existing variants thereof (e.g., allotypes). CH1 domain sequences of IgG1, IgG2, IgG3, and IgG4 (including wildtype and allotypes) are known in the art. See, e.g., Kabat E A et al., (1991) supra and Vidarsson G. et al., Front Immunol. 5:520 (published online Oct. 20, 2014). Exemplary CH1 domains include CH1 domains with mutations that modify a biological activity of an antibody, e.g., half-life, e.g., described in U.S. Pub. No. 20120100140 and U.S. patents and publications and PCT publications cited therein.

The term “CH2 domain” refers to the heavy chain constant region linking the hinge to the CH3 domain in a heavy chain constant domain. As used herein, a CH2 domain starts at P238 and ends at K340. The term “CH2 domain” includes wildtype CH2 domains, as well as naturally existing variants thereof (e.g., allotypes). CH2 domain sequences of IgG1, IgG2, IgG3, and IgG4 (including wildtype and allotypes) are known in the art. See, e.g., Kabat E A et al., (1991) supra and Vidarsson G. et al., Front Immunol. 5:520 (published online Oct. 20, 2014). Exemplary CH2 domains include CH2 domains with mutations that modify a biological activity of an antibody, e.g., half-life and/or reduced Fc effector function, e.g., described in U.S. Pub. No. 20120100140 and U.S. patents and publications and PCT publications cited therein.

The term “CH3 domain” refers to the heavy chain constant region that is C-terminal to the CH2 domain in a heavy chain constant domain. As used herein, a CH3 domain starts at G341 and ends at K447. The term “CH3 domain” includes wildtype CH3 domains, as well as naturally existing variants thereof (e.g., allotypes). CH3 domain sequences of IgG1, IgG2, IgG3, and IgG4 (including wildtype and allotypes) are known in the art. See, e.g., Kabat E A et al., (1991) supra and Vidarsson G. et al., Front Immunol. 5:520 (published online Oct. 20, 2014). Exemplary CH3 domains include CH3 domains with mutations that modify a biological activity of an antibody, e.g., half-life, e.g., described in U.S. Pub. No. 20120100140 and U.S. patents and publications and PCT publications cited therein.

As used herein, “isotype” refers to the antibody class (e.g., IgG1, IgG2, IgG3, IgG4, IgM, IgAQ1 IgA2, IgD, and IgE antibody) that is encoded by the heavy chain constant region genes.

“Allotype” refers to naturally occurring variants within a specific isotype group, which variants differ in a few amino acids (see, e.g., Jefferis et al., (2009) mAbs 1:1). Antibodies described herein can be of any allotype. Allotypes of IgG1, IgG2, IgG3, and IgG4 are known in the art. See, e.g., Kabat E A et al., (1991) supra; Vidarsson G. et al., Front Immunol. 5:520 (published online Oct. 20, 2014); and Lefranc M P, mAbs 1:4, 1-7(2009).

The phrases “an antibody recognizing an antigen” and “an antibody specific for an antigen” are used interchangeably herein with the term “an antibody which binds specifically to an antigen.”

As used herein, “anti-idiotypic” or “anti-idiotypic antibody” refers to an antibody raised against a first/primary antibody which specifically binds to an idiotope of the first antibody. As used herein, the term “idiotope” or “idiotypic determinant” refers to an antigenic determinant or epitope expressed on a first/primary antibody. In some embodiments, the idiotope is unique to the immunoglobulin product of a single clone of cells. In some embodiments, the idiotope is found in the variable region of the primary antibody. In some embodiments, the idiotope is a framework-associated or regulatory idiotope in the primary antibody.

An “isolated antibody,” as used herein, is intended to refer to an antibody which is substantially free of other antibodies having different antigenic specificities (e.g., an isolated antibody that specifically binds to B. burgdorferi peptidoglycan is substantially free of antibodies that specifically bind antigens other than B. burgdorferi peptidoglycan).

“Binding affinity” generally refers to the strength of the sum total of non-covalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{D}). Affinity can be measured and/or expressed in a number of ways known in the art, including, but not limited to, equilibrium dissociation constant (K_{D}), and equilibrium association constant (K_{A}). The K_{D} is calculated from the quotient of k_{off}/kₒₙ and is expressed as a molar concentration (M), whereas K_{A} is calculated from the quotient of kₒₙ/k_{off}. kₒₙ refers to the association rate constant of, e.g., an antibody to an antigen, and k_{off} refers to the dissociation of, e.g., an antibody to an antigen. The kₒₙ and k_{off} can be determined by techniques known to one of ordinary skill in the art, such as immunoassays (e.g., enzyme-linked immunosorbent assay (ELISA)), BIACORE® or kinetic exclusion assay (KinExA).

As used herein, the terms “specifically binds,” “specifically recognizes,” “specific binding,” “selective binding,” and “selectively binds,” are analogous terms in the context of antibodies and refer to molecules (e.g., antibodies) that bind to an antigen (e.g., epitope or immune complex) as such binding is understood by one skilled in the art. For example, a molecule that specifically binds to an antigen can bind to other peptides or polypeptides, generally with lower affinity as determined by, e.g., immunoassays, BIACORE®, KinExA 3000 instrument (Sapidyne Instruments, Boise, ID), or other assays known in the art. In a specific embodiment, molecules that specifically bind to an antigen bind to the antigen with a K_{A} that is at least 2 logs, 2.5 logs, 3 logs, 4 logs or greater than the K_{A} when the molecules bind to another antigen.

As used herein, the term “binds to a B. burgdorferi peptidoglycan” refers to the binding of, e.g., an anti-peptidoglycan antibody disclosed herein, to one or more moieties or epitopes on a Borrelia burgdorferi peptidoglycan. The B. burgdorferi peptidoglycan can be a whole peptidoglycan or a fragment/remnant thereof.

Antibodies typically bind specifically to their cognate antigen with high affinity, reflected by a dissociation constant (K_{D}) of 10⁻⁵ to 10⁻¹¹ M or less. Any K_{D} greater than about 10⁻⁴ M is generally considered to indicate nonspecific binding. As used herein, an antibody that “binds specifically” to an antigen refers to an antibody that binds to the antigen and substantially identical antigens with high affinity, which means having a K_{D} of 10⁻⁷ M or less, preferably 10⁻⁸ M or less, even more preferably 10⁻⁹ M or less, and most preferably between 10⁻⁸ M and 10⁻¹⁰ M or less, when determined by, e.g., immunoassays (e.g., ELISA) or surface plasmon resonance (SPR) technology in a BIACORE 2000 instrument using the predetermined antigen, but does not bind with high affinity to unrelated antigens.

Unless specified otherwise, the term “anti-peptidoglycan antibody” used herein refers to an antibody that specifically binds to peptidoglycan of B. burgdorferi.

As used herein, the term “antigen” refers to any natural or synthetic immunogenic substance, such as a protein, peptide, or hapten. In some embodiments, an antigen comprises B. burgdorferi peptidoglycan. In other embodiments, an antigen comprises an antibody (e.g., variable region) directed against B. burgdorferi peptidoglycan (e.g., anti-idiotypic antibody).

As used herein, an “epitope” is a term in the art and refers to a localized region of an antigen to which an antibody can specifically bind. An epitope can be, for example, contiguous amino acids of a polypeptide (linear or contiguous epitope) or an epitope can, for example, come together from two or more non-contiguous regions of a polypeptide or polypeptides (conformational, non-linear, discontinuous, or non-contiguous epitope). Epitopes formed from contiguous amino acids are typically, but not always, retained on exposure to denaturing solvents, whereas epitopes formed by tertiary folding are typically lost on treatment with denaturing solvents. An epitope typically includes at least 3, 4, 5, 6, 7, 8, 9, 10, 11, 12, 13, 14, 15, or 20 amino acids in a unique spatial conformation. Methods for determining what epitopes are bound by a given antibody (i.e., epitope mapping) are well known in the art. Methods of determining spatial conformation of epitopes include techniques in the art and those described herein, for example, x-ray crystallography, 2-dimensional nuclear magnetic resonance and HDX-MS (see, e.g., Epitope Mapping Protocols in Methods in Molecular Biology, Vol. 66, G. E. Morris, Ed. (1996)).

In certain embodiments, the epitope to which an antibody binds can be determined by, e.g., NMR spectroscopy, X-ray diffraction crystallography studies, ELISA assays, hydrogen/deuterium exchange coupled with mass spectrometry (e.g., liquid chromatography electrospray mass spectrometry), array-based oligo-peptide scanning assays, and/or mutagenesis mapping (e.g., site-directed mutagenesis mapping). For X-ray crystallography, crystallization can be accomplished using any of the known methods in the art (e.g., Giege R et al., (1994) Acta Crystallogr D Biol Crystallogr 50 (Pt 4): 339-350; McPherson A (1990) Eur J Biochem 189:1-23; Chayen N E (1997) Structure 5:1269-1274; McPherson A (1976) J Biol Chem 251:6300-6303). Antibody: antigen crystals can be studied using well known X-ray diffraction techniques and can be refined using computer software such as X-PLOR (Yale University, 1992, distributed by Molecular Simulations, Inc.; see, e.g., Meth Enzymol (1985) volumes 114 & 115, eds Wyckoff H W et al.; U.S. 2004/0014194), and BUSTER (Bricogne G (1993) Acta Crystallogr D Biol Crystallogr 49 (Pt 1): 37-60; Bricogne G (1997) Meth Enzymol 276A: 361-423, ed Carter C W; Roversi P et al., (2000) Acta Crystallogr D Biol Crystallogr 56 (Pt 10): 1316-1323). Mutagenesis mapping studies can be accomplished using any method known to one of skill in the art. See, e.g., Champe M et al., (1995) J Biol Chem 270:1388-1394 and Cunningham B C & Wells J A (1989) Science 244:1081-1085 for a description of mutagenesis techniques, including alanine scanning mutagenesis techniques.

The term “binds to the same epitope” with reference to two or more antibodies means that the antibodies bind to the same segment of amino acid residues, as determined by a given method. Techniques for determining whether antibodies bind to the same epitope with the antibodies described herein include, for example, epitope mapping methods, such as, x-ray analyses of crystals of antigen: antibody complexes which provides atomic resolution of the epitope and hydrogen/deuterium exchange mass spectrometry (HDX-MS). Other methods monitor the binding of the antibody to antigen fragments or mutated variations of the antigen where loss of binding due to a modification of an amino acid residue within the antigen sequence is often considered an indication of an epitope component. In addition, computational combinatorial methods for epitope mapping can also be used. These methods rely on the ability of the antibody of interest to affinity isolate specific short peptides from combinatorial phage display peptide libraries. Antibodies having the same VH and VL or the same CDR1, 2 and 3 sequences are expected to bind to the same epitope.

Antibodies that “compete with another antibody for binding to a target” refer to antibodies that inhibit (partially or completely) the binding of the other antibody to the target. Whether two antibodies compete with each other for binding to a target, i.e., whether and to what extent one antibody inhibits the binding of the other antibody to a target, can be determined using known competition experiments. In certain embodiments, an antibody competes with, and inhibits binding of another antibody to a target by at least 10%, 20%, 30%, 40%, 50%, 60%, 70%, 80%, 90% or 100%. The level of inhibition or competition can be different depending on which antibody is the “blocking antibody” (i.e., the cold antibody that is incubated first with the target). Competition assays can be conducted as described, for example, in Ed Harlow and David Lane, Cold Spring Harb Protoc; 2006; doi: 10.1101/pdb.prot4277 or in Chapter 11 of “Using Antibodies” by Ed Harlow and David Lane, Cold Spring Harbor Laboratory Press, Cold Spring Harbor, NY, USA 1999. Competing antibodies bind to the same epitope, an overlapping epitope or to adjacent epitopes (e.g., as evidenced by steric hindrance).

Other competitive binding assays include: solid phase direct or indirect radioimmunoassay (RIA), solid phase direct or indirect enzyme immunoassay (EIA), sandwich competition assay (see Stahli et al., Methods in Enzymology 9:242 (1983)); solid phase direct biotin-avidin EIA (see Kirkland et al., J. Immunol. 137:3614 (1986)); solid phase direct labeled assay, solid phase direct labeled sandwich assay (see Harlow and Lane, Antibodies: A Laboratory Manual, Cold Spring Harbor Press (1988)); solid phase direct label RIA using 1-125 label (see Morel et al., Mol. Immunol. 25(1):7(1988)); solid phase direct biotin-avidin EIA (Cheung et al., Virology 176:546 (1990)); and direct labeled RIA. (Moldenhauer et al., Scand. J. Immunol. 32:77 (1990)).

The term “monoclonal antibody,” as used herein, refers to an antibody that displays a single binding specificity and affinity for a particular epitope or a composition of antibodies in which all antibodies display a single binding specificity and affinity for a particular epitope. Accordingly, the term “human monoclonal antibody” refers to an antibody or antibody composition that display(s) a single binding specificity and which has variable and optional constant regions derived from human germline immunoglobulin sequences. In one embodiment, human monoclonal antibodies are produced by a hybridoma which includes a B cell obtained from a transgenic non-human animal, e.g., a transgenic mouse, having a genome comprising a human heavy chain transgene and a light chain transgene fused to an immortalized cell.

The term “recombinant human antibody,” as used herein, includes all human antibodies that are prepared, expressed, created or isolated by recombinant means, such as (a) antibodies isolated from an animal (e.g., a mouse) that is transgenic or trans-chromosomal for human immunoglobulin genes or a hybridoma prepared therefrom, (b) antibodies isolated from a host cell transformed to express the antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies comprise variable and constant regions that utilize particular human germline immunoglobulin sequences are encoded by the germline genes, but include subsequent rearrangements and mutations which occur, for example, during antibody maturation. As known in the art (see, e.g., Lonberg (2005) Nature Biotech. 23(9): 1117-1125), the variable region contains the antigen binding domain, which is encoded by various genes that rearrange to form an antibody specific for a foreign antigen. In addition to rearrangement, the variable region can be further modified by multiple single amino acid changes (referred to as somatic mutation or hypermutation) to increase the affinity of the antibody to the foreign antigen. The constant region will change in further response to an antigen (i.e., isotype switch). Therefore, the rearranged and somatically mutated nucleic acid molecules that encode the light chain and heavy chain immunoglobulin polypeptides in response to an antigen cannot have sequence identity with the original nucleic acid molecules, but instead will be substantially identical or similar (i.e., have at least 80% identity).

A “human” antibody (HuMAb) refers to an antibody having variable regions in which both the framework and CDR regions are derived from human germline immunoglobulin sequences. Furthermore, if the antibody contains a constant region, the constant region also is derived from human germline immunoglobulin sequences. The antibodies described herein can include amino acid residues not encoded by human germline immunoglobulin sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences. The terms “human” antibodies and “fully human” antibodies and are used synonymously.

A “humanized” antibody refers to an antibody in which some, most or all of the amino acids outside the CDR domains of a non-human antibody are replaced with corresponding amino acids derived from human immunoglobulins. In one embodiment of a humanized form of an antibody, some, most or all of the amino acids outside the CDR domains have been replaced with amino acids from human immunoglobulins, whereas some, most or all amino acids within one or more CDR regions are unchanged. Small additions, deletions, insertions, substitutions or modifications of amino acids are permissible as long as they do not abrogate the ability of the antibody to bind to a particular antigen. A “humanized” antibody retains an antigenic specificity similar to that of the original antibody.

As used herein, the term “de-immunized” or “de-immunization” refers to a process in which an antibody, or an antigen binding portion thereof, is modified to reduce its immunogenicity, e.g., in a human subject. For example, the heavy chain variable region (VH) and the light chain variable region (VL) sequences from the original antibody can be analyzed and a human T cell epitope “map” can be generated from each V region showing the location of epitopes in relation to complementarity-determining regions (CDRs) and other key residues within the sequence. Individual T cell epitopes from the T cell epitope map are analyzed in order to identify alternative amino acid substitutions with a low risk of altering activity of the final antibody. A range of alternative VH and VL sequences are designed comprising combinations of amino acid substitutions and these sequences are subsequently incorporated into a range of Borreliae peptidoglycan-specific antibodies, or antigen binding portion thereof, for use in the diagnostic and treatment methods disclosed herein, which are then tested for function. Complete heavy and light chain genes comprising modified V and human C regions are then cloned into expression vectors and the subsequent plasmids introduced into cell lines for the production of whole antibody. The antibodies are then compared in appropriate biochemical and biological assays, and the optimal variant is identified. An antibody can be de-immunized by using methods described herein or by any other methods known in the art. See, e.g., WO 98/52976 or WO 00/34317.

A “chimeric antibody” refers to an antibody in which the variable regions are derived from one species and the constant regions are derived from another species, such as an antibody in which the variable regions are derived from a mouse antibody and the constant regions are derived from a human antibody.

### Encoding Polynucleotides

Described in several example embodiments herein are polynucleotide(s) that encode an antibody or fragment thereof capable of specifically binding a Borreliae peptidoglycan. In certain example embodiments, the polynucleotide(s) encode the antibody or fragment thereof described herein that is capable of binding a glycan component of the Borrelia peptidoglycan, an alpha-Orn linkage in a Borreliae peptidoglycan, or a gamma-Orn linkage in a Borreliae peptidoglycan. In certain example embodiments, the polynucleotide(s) encode the antibody or fragment thereof described herein comprising one or more of SEQ ID NO: 1-8. In certain example embodiments, the polynucleotide(s) encoding the antibody or fragment thereof comprise one or more of SEQ ID NO: 9-16. Described in several example embodiments herein are engineered polynucleotide(s) that encode a polypeptide having a sequence according to any one of SEQ ID NO: 1-8. Described in several example embodiments herein are engineered polynucleotide(s) that encode a polypeptide having a sequence that is 80%-100% identical to any one of SEQ ID NO: 1-8, such as 80%, to/or 81%, 82%, 83%, 84%, 85%, 86%, 87%, 88%, 89%, 90%, 91%, 92%, 93%, 94%, 95%, 96%, 97%, 98%, 99%, 100% identical to any one of SEQ ID NO: 1-8. In some embodiments, the engineered polynucleotide is codon optimized for expression in specific cell or tissue type. In some embodiments, the engineered polynucleotide is codon optimized for expression in specific species or organism. Codon optimization is described in further detail elsewhere herein. The engineered polynucleotides can be DNA or RNA or DNA/RNA hybrids.

Described in several example embodiments herein are engineered polynucleotide(s) each including or being entirely composed of a sequence that is 70-100% identical to any one of SEQ ID NO: 9-16, 70%, to/or 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100% identical to any one of SEQ ID NO: 9-16. In some embodiments, the engineered polynucleotide is codon optimized for expression in specific cell or tissue type. In some embodiments, the engineered polynucleotide is codon optimized for expression in specific species or organism. Codon optimization is described in further detail elsewhere herein. The engineered polynucleotides can be DNA or RNA or DNA/RNA hybrids.

In certain example embodiments, the polynucleotide(s) encode the antibody or fragment thereof described herein comprising a chimeric antibody, a human antibody, or a humanized antibody. In certain example embodiments, the polynucleotide(s) encode the antibody or fragment thereof described herein comprising a Fab, a Fab′, a F(ab′)2, a Fv, a single chain Fv (scFv), or combinations thereof. In certain example embodiments, the polynucleotide(s) encode the antibody or fragment thereof described herein selected from the group consisting of an IgG1, an IgG2, an IgG3, an IgG4, a variant thereof, and any combination thereof. In certain example embodiments, the polynucleotide(s) encode the antibody or fragment thereof described herein linked to an agent, thereby forming an immunoconjugate. In certain example embodiments, the polynucleotide(s) encode the antibody or fragment described herein wherein the antibody or fragment thereof is a monoclonal antibody, optionally selected from 1A4-1, 1E10, 1G11, or 2H10. In some embodiments, the polynucleotide is codon optimized for expression in specific cell or tissue type. In some embodiments, the polynucleotide is codon optimized for expression in specific species or organism. Codon optimization is described in further detail elsewhere herein. The polynucleotides can be DNA or RNA or DNA/RNA hybrids.

### Vectors and Vector Systems

Also provided herein are vectors that can contain one or more of the engineered polynucleotides described herein, such as those encoding an antibody or fragment thereof of the present disclosure. In some embodiments, one or more of the engineered polynucleotides(s) are operatively coupled to one or more regulatory elements, such as promoters. The vectors can be useful in producing bacterial, fungal, yeast, plant cells, animal cells, and transgenic animals that can express and/or produce one or more antibodies or fragments thereof of the present disclosure. The vectors and/or vector systems can be used, for example, to express one or more of the polynucleotides in a cell, such as a producer cell, to produce virus particles described elsewhere herein or to produce antibodies or fragments thereof. Other uses for the vectors and vector systems described herein are also within the scope of this disclosure. In general, and throughout this specification, the term “vector” refers to a tool that allows or facilitates the transfer of an entity from one environment to another. In some contexts which will be appreciated by those of ordinary skill in the art, “vector” can be a term of art to refer to a nucleic acid molecule capable of transporting another nucleic acid to which it has been linked. A vector can be a replicon, such as a plasmid, phage, or cosmid, into which another DNA segment may be inserted so as to bring about the replication of the inserted segment. Generally, a vector is capable of replication when associated with the proper control elements.

Vectors include, but are not limited to, nucleic acid molecules that are single-stranded, double-stranded, or partially double-stranded; nucleic acid molecules that comprise one or more free ends, no free ends (e.g., circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g. retroviruses, replication defective retroviruses, adenoviruses, replication defective adenoviruses, and adeno-associated viruses (AAVs)). Viral vectors also include polynucleotides carried by a virus for transfection into a host cell. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Common expression vectors of utility in recombinant DNA techniques are often in the form of plasmids.

Recombinant expression vectors can be composed of a nucleic acid (e.g., a polynucleotide) of the invention in a form suitable for expression of the nucleic acid in a host cell, which means that the recombinant expression vectors include one or more regulatory elements, which can be selected on the basis of the host cells to be used for expression, that is operatively-linked to the nucleic acid sequence to be expressed. Within a recombinant expression vector, “operably linked” and “operatively-linked” are used interchangeably herein and further defined elsewhere herein. In the context of a vector, the term “operably linked” is intended to mean that the nucleotide sequence of interest is linked to the regulatory element(s) in a manner that allows for expression of the nucleotide sequence (e.g., in an in vitro transcription/translation system or in a host cell when the vector is introduced into the host cell). Advantageous vectors include lentiviruses and adeno-associated viruses, and types of such vectors can also be selected for targeting particular types of cells. These and other embodiments of the vectors and vector systems are described elsewhere herein.

### Cell-Based Vector Amplification and Expression

Vectors may be introduced and propagated in a prokaryote or prokaryotic cell. In some embodiments, a prokaryote is used to amplify copies of a vector to be introduced into a eukaryotic cell or as an intermediate vector in the production of a vector to be introduced into a eukaryotic cell (e.g., amplifying a plasmid as part of a viral vector packaging system). The vectors can be viral-based or non-viral based. In some embodiments, a prokaryote is used to amplify copies of a vector and express one or more nucleic acids, such as to provide a source of one or more proteins for delivery to a host cell or host organism.

Vectors can be designed for expression of an antibody or fragment thereof in a suitable host cell. In some embodiments, the suitable host cell is a prokaryotic cell. Suitable host cells include, but are not limited to, bacterial cells, yeast cells, insect cells, and mammalian cells. In some embodiments, the suitable host cell is a eukaryotic cell.

In some embodiments, the suitable host cell is a suitable bacterial cell. Suitable bacterial cells include, but are not limited to, bacterial cells from the bacteria of the species Escherichia coli. Many suitable strains of E. coli are known in the art for expression of vectors. These include, but are not limited to Pir1, Stb12, Stb13, Stb14, TOP10, XL1 Blue, and XL10 Gold. In some embodiments, the host cell is a suitable insect cell. Suitable insect cells include those from Spodoptera frugiperda. Suitable strains of S. frugiperda cells include, but are not limited, to Sf9 and Sf21. In some embodiments, the host cell is a suitable yeast cell. In some embodiments, the yeast cell can be from Saccharomyces cerevisiae. In some embodiments, the host cell is a suitable mammalian cell. Many types of mammalian cells have been developed to express vectors. Suitable mammalian cells include, but are not limited to, HEK293, Chinese Hamster Ovary Cells (CHOs), mouse myeloma cells, HeLa, U2OS, A549, HT1080, CAD, P19, NIH 3T3, L929, N2a, MCF-7, Y79, SO-Rb50, HepG G2, DIKX-X11, J558L, Baby hamster kidney cells (BHK), and chicken embryo fibroblasts (CEFs). Suitable host cells are discussed further in Goeddel, GENE EXPRESSION TECHNOLOGY: METHODS IN ENZYMOLOGY 185, Academic Press, San Diego, Calif. (1990).

In some embodiments, the vector can be a yeast expression vector. Examples of vectors for expression in yeast Saccharomyces cerevisiae include pYepSec1 (Baldari, et al., 1987. EMBO J. 6:229-234), pMFa (Kuijan and Herskowitz, 1982. Cell 30:933-943), pJRY88 (Schultz et al., 1987. Gene 54:113-123), pYES2 (Invitrogen Corporation, San Diego, Calif.), and picZ (Invitrogen Corp, San Diego, Calif.). As used herein, a “yeast expression vector” refers to a nucleic acid that contains one or more sequences encoding an RNA and/or polypeptide and may further contain any desired elements that control the expression of the nucleic acid(s), as well as any elements that enable the replication and maintenance of the expression vector inside the yeast cell. Many suitable yeast expression vectors and features thereof are known in the art; for example, various vectors and techniques are illustrated in in Yeast Protocols, 2nd edition, Xiao, W., ed. (Humana Press, New York, 2007) and Buckholz, R. G. and Gleeson, M. A. (1991) Biotechnology (NY) 9(11): 1067-72. Yeast vectors can contain, without limitation, a centromeric (CEN) sequence, an autonomous replication sequence (ARS), a promoter, such as an RNA Polymerase III promoter, operably linked to a sequence or gene of interest, a terminator such as an RNA polymerase III terminator, an origin of replication, and a marker gene (e.g., auxotrophic, antibiotic, or other selectable markers). Examples of expression vectors for use in yeast may include plasmids, yeast artificial chromosomes, 2μ plasmids, yeast integrative plasmids, yeast replicative plasmids, shuttle vectors, and episomal plasmids.

In some embodiments, the vector is a baculovirus vector or expression vector and can be suitable for expression of polynucleotides and/or proteins in insect cells. In some embodiments, the suitable host cell is an insect cell. Baculovirus vectors available for expression of proteins in cultured insect cells (e.g., SF9 cells) include the pAc series (Smith, et al., 1983. Mol. Cell. Biol. 3:2156-2165) and the pVL series (Lucklow and Summers, 1989. Virology 170:31-39). rAAV (recombinant Adeno-associated viral) vectors are preferably produced in insect cells, e.g., Spodoptera frugiperda Sf9 insect cells, grown in serum-free suspension culture. Serum-free insect cells can be purchased from commercial vendors, e.g., Sigma Aldrich (EX-CELL 405).

In some embodiments, the vector is a mammalian expression vector. In some embodiments, the mammalian expression vector is capable of expressing one or more polynucleotides and/or polypeptides in a mammalian cell. Examples of mammalian expression vectors include, but are not limited to, pCDM8 (Seed, 1987. Nature 329:840) and pMT2PC (Kaufman, et al., 1987. EMBO J. 6:187-195). The mammalian expression vector can include one or more suitable regulatory elements capable of controlling expression of the one or more polynucleotides and/or proteins in the mammalian cell. For example, commonly used promoters are derived from polyoma, adenovirus 2, cytomegalovirus, simian virus 40, and others disclosed herein and known in the art. More detail on suitable regulatory elements are described elsewhere herein.

For other suitable expression vectors and vector systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In some embodiments, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1:268-277), lymphoid-specific promoters (Calame and Eaton, 1988. Adv. Immunol. 43:235-275), in particular promoters of T cell receptors (Winoto and Baltimore, 1989. EMBO J. 8:729-733) and immunoglobulins (Baneiji, et al., 1983. Cell 33:729-740; Queen and Baltimore, 1983. Cell 33:741-748), neuron-specific promoters (e.g., the neurofilament promoter; Byrne and Ruddle, 1989. Proc. Natl. Acad. Sci. USA 86:5473-5477), pancreas-specific promoters (Edlund, et al., 1985. Science 230:912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249:374-379) and the α-fetoprotein promoter (Campes and Tilghman, 1989. Genes Dev. 3:537-546). With regards to these prokaryotic and eukaryotic vectors, mention is made of U.S. Pat. No. 6,750,059, the contents of which are incorporated by reference herein in their entirety. Other embodiments can utilize viral vectors, with regards to which mention is made of U.S. patent application Ser. No. 13/092,085, the contents of which are incorporated by reference herein in their entirety. Tissue-specific regulatory elements are known in the art and in this regard, mention is made of U.S. Pat. No. 7,776,321, the contents of which are incorporated by reference herein in their entirety. In some embodiments, a regulatory element can be operably linked to one or more of the engineered polynucleotides of the present disclosure so as to drive expression of the of the engineered polynucleotides of the present disclosure.

In some embodiments, the vector can be a fusion vector or fusion expression vector. In some embodiments, fusion vectors add a number of amino acids to a protein encoded therein, such as to the amino terminus, carboxy terminus, or both of a recombinant protein. Such fusion vectors can serve one or more purposes, such as: (i) to increase expression of recombinant protein; (ii) to increase the solubility of the recombinant protein; and (iii) to aid in the purification of the recombinant protein by acting as a ligand in affinity purification. In some embodiments, expression of polynucleotides (such as non-coding polynucleotides) and proteins in prokaryotes can be carried out in Escherichia coli with vectors containing constitutive or inducible promoters directing the expression of either fusion or non-fusion polynucleotides and/or proteins. In some embodiments, the fusion expression vector can include a proteolytic cleavage site, which can be introduced at the junction of the fusion vector backbone or other fusion moiety and the recombinant polynucleotide or protein to enable separation of the recombinant polynucleotide or protein from the fusion vector backbone or other fusion moiety subsequent to purification of the fusion polynucleotide or protein. Such enzymes, and their cognate recognition sequences, include Factor Xa, thrombin and enterokinase. Example fusion expression vectors include pGEX (Pharmacia Biotech Inc; Smith and Johnson, 1988. Gene 67:31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein. Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

### Cell-Free Vector and Polynucleotide Expression

In some embodiments, the polynucleotide encoding an antibody or fragment thereof of the present disclosure is expressed from a vector or suitable polynucleotide in a cell-free in vitro system. In other words, the polynucleotide can be transcribed and optionally translated in vitro. In vitro transcription/translation systems and appropriate vectors are generally known in the art and commercially available. Generally, in vitro transcription and in vitro translation systems replicate the processes of RNA and protein synthesis, respectively, outside of the cellular environment. Vectors and suitable polynucleotides for in vitro transcription can include T7, SP6, T3, promoter regulatory sequences that can be recognized and acted upon by an appropriate polymerase to transcribe the polynucleotide or vector.

In vitro translation can be stand-alone (e.g., translation of a purified polyribonucleotide) or linked/coupled to transcription. In some embodiments, the cell-free (or in vitro) translation system can include extracts from rabbit reticulocytes, wheat germ, and/or E. coli. The extracts can include various macromolecular components that are needed for translation of exogenous RNA (e.g., 70S or 80S ribosomes, tRNAs, aminoacyl-tRNA, synthetases, initiation, elongation factors, termination factors, etc.). Other components can be included or added during the translation reaction, including but not limited to, amino acids, energy sources (ATP, GTP), energy regenerating systems (creatine phosphate and creatine phosphokinase (eukaryotic systems)) (phosphoenol pyruvate and pyruvate kinase for bacterial systems), and other co-factors (Mg2+, K+, etc.). As previously mentioned, in vitro translation can be based on RNA or DNA starting material. Some translation systems can utilize an RNA template as starting material (e.g., reticulocyte lysates and wheat germ extracts). Some translation systems can utilize a DNA template as a starting material (e.g., E coli-based systems). In these systems transcription and translation are coupled and DNA is first transcribed into RNA, which is subsequently translated.

Suitable standard and coupled cell-free translation systems are generally known in the art and are commercially available.

### Vector Features

The vectors can include additional features that can confer one or more functionalities to the vector, the polynucleotide to be delivered, a virus particle produced there from, or polypeptide expressed thereof. Such features include, but are not limited to, regulatory elements, selectable markers, molecular identifiers (e.g., molecular barcodes), stabilizing elements, and the like. It will be appreciated by those skilled in the art that the design of the expression vector and additional features included can depend on such factors as the choice of the host cell to be transformed, the level of expression desired, etc.

### Regulatory Elements

In certain embodiments, the engineered polynucleotides and/or vectors thereof described herein can include one or more regulatory elements that can be operatively linked to the polynucleotide. The term “regulatory element” is intended to include promoters, enhancers, internal ribosomal entry sites (IRES), other expression control elements (e.g., transcription termination signals, such as polyadenylation signals and poly-U sequences) and cellular localization signals (e.g., nuclear localization signals). Such regulatory elements are described, for example, in Goeddel, GENE EXPRESSION TECHNOLOGY: METHODS IN ENZYMOLOGY 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter can direct expression primarily in a desired tissue of interest, such as muscle, neuron, bone, skin, blood, specific organs (e.g., liver, pancreas), or particular cell types (e.g., lymphocytes). Regulatory elements may also direct expression in a temporal-dependent manner, such as in a cell-cycle dependent or developmental stage-dependent manner, which may or may not also be tissue or cell-type specific. In some embodiments, a vector comprises one or more pol III promoter (e.g., 1, 2, 3, 4, 5, or more pol III promoters), one or more pol II promoters (e.g., 1, 2, 3, 4, 5, or more pol II promoters), one or more pol I promoters (e.g., 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) (see, e.g., Boshart et al, Cell, 41:521-530 (1985)), the SV40 promoter, the dihydrofolate reductase promoter, the β-actin promoter, the phosphoglycerol kinase (PGK) promoter, and the EF1α promoter. Also encompassed by the term “regulatory element” are enhancer elements, such as WPRE; CMV enhancers; the R-U5′ segment in LTR of HTLV-I (Mol. Cell. Biol., Vol. 8(1), p. 466-472, 1988); SV40 enhancer; and the intron sequence between exons 2 and 3 of rabbit β-globin (Proc. Natl. Acad. Sci. USA., Vol. 78(3), p. 1527-31, 1981).

In some embodiments, the regulatory sequence can be a regulatory sequence described in U.S. Pat. No. 7,776,321, U.S. Pat. Pub. No. 2011/0027239, and International Patent Publication No. WO 2011/028929, the contents of which are incorporated by reference herein in their entirety. In some embodiments, the vector can contain a minimal promoter. In some embodiments, the minimal promoter is the Mecp2 promoter, tRNA promoter, or U6. In a further embodiment, the minimal promoter is tissue specific.

To express a polynucleotide, the vector can include one or more transcriptional and/or translational initiation regulatory sequences, e.g., promoters, that direct the transcription of the gene and/or translation of the encoded protein in a cell. In some embodiments a constitutive promoter may be employed. Suitable constitutive promoters for mammalian cells are generally known in the art and include, but are not limited to SV40, CAG, CMV, EF-1α, β-actin, RSV, and PGK. Suitable constitutive promoters for bacterial cells, yeast cells, and fungal cells are generally known in the art, such as a T-7 promoter for bacterial expression and an alcohol dehydrogenase promoter for expression in yeast.

In some embodiments, the regulatory element can be a regulated promoter. “Regulated promoter” refers to promoters that direct gene expression not constitutively, but in a temporally- and/or spatially-regulated manner, and includes tissue-specific, tissue-preferred and inducible promoters. Regulated promoters include conditional promoters and inducible promoters. In some embodiments, conditional promoters can be employed to direct expression of a polynucleotide in a specific cell type, under certain environmental conditions, and/or during a specific state of development. Suitable tissue specific promoters can include, but are not limited to, liver specific promoters (e.g. APOA2, SERPIN A1 (hAAT), CYP3A4, and MIR122), pancreatic cell promoters (e.g. INS, IRS2, Pdx1, Alx3, Ppy), cardiac specific promoters (e.g. Myh6 (alpha MHC), MYL2 (MLC-2v), TNI3 (cTnl), NPPA (ANF), Slc8a1 (Ncx1)), central nervous system cell promoters (SYN1, GFAP, INA, NES, MOBP, MBP, TH, FOXA2 (HNF3 beta)), skin cell specific promoters (e.g. FLG, K14, TGM3), immune cell specific promoters, (e.g. ITGAM, CD43 promoter, CD14 promoter, CD45 promoter, CD68 promoter), urogenital cell specific promoters (e.g. Pbsn, Upk2, Sbp, Fer114), endothelial cell specific promoters (e.g. ENG), pluripotent and embryonic germ layer cell specific promoters (e.g. Oct4, NANOG, Synthetic Oct4, T brachyury, NES, SOX17, FOXA2, MIR122), and muscle cell specific promoter (e.g. Desmin). Other tissue and/or cell specific promoters are generally known in the art and are within the scope of this disclosure.

Inducible/conditional promoters can be positively inducible/conditional promoters (e.g. a promoter that activates transcription of the polynucleotide upon appropriate interaction with an activated activator, or an inducer (compound, environmental condition, or other stimulus) or a negative/conditional inducible promoter (e.g., a promoter that is repressed (e.g., bound by a repressor) until the repressor condition of the promotor is removed (e.g., inducer binds a repressor bound to the promoter stimulating release of the promoter by the repressor or removal of a chemical repressor from the promoter environment). The inducer can be a compound, environmental condition, or other stimulus. Thus, inducible/conditional promoters can be responsive to any suitable stimuli such as chemical, biological, or other molecular agents, temperature, light, and/or pH. Suitable inducible/conditional promoters include, but are not limited to, Tet-On, Tet-Off, Lac promoter, pBad, AlcA, LexA, Hsp70 promoter, Hsp90 promoter, pDawn, XVE/OlexA, GVG, and pOp/LhGR.

Where expression in a plant cell is desired, the engineered polynucleotide(s) can be placed under control of a plant promoter, i.e., a promoter operable in plant cells. The use of different types of promoters is envisaged. A constitutive plant promoter is a promoter that is able to express the open reading frame (ORF) that it controls in all or nearly all of the plant tissues during all or nearly all developmental stages of the plant (referred to as “constitutive expression”). One non-limiting example of a constitutive promoter is the cauliflower mosaic virus 35S promoter. Different promoters may direct the expression of a gene in different tissues or cell types, or at different stages of development, or in response to different environmental conditions. In particular embodiments, one or more of the engineered polynucleotides are expressed under the control of a constitutive promoter, such as the cauliflower mosaic virus 35S promoter issue-preferred promoters can be utilized to target enhanced expression in certain cell types within a particular plant tissue, for instance vascular cells in leaves or roots or in specific cells of the seed. Examples of particular promoters for use for expression of the engineered polynucleotide(s) are found in Kawamata et al., (1997) Plant Cell Physiol 38:792-803; Yamamoto et al., (1997) Plant J 12:255-65; Hire et al, (1992) Plant Mol Biol 20:207-18, Kuster et al, (1995) Plant Mol Biol 29:759-72, and Capana et al., (1994) Plant Mol Biol 25:681-91.

Examples of promoters that are inducible and that can allow for spatiotemporal control of gene editing or gene expression may use a form of energy. The form of energy may include but is not limited to sound energy, electromagnetic radiation, chemical energy and/or thermal energy. Examples of inducible systems include tetracycline inducible promoters (Tet-On or Tet-Off), small molecule two-hybrid transcription activations systems (FKBP, ABA, etc.), or light inducible systems (Phytochrome, LOV domains, or cryptochrome), such as a Light Inducible Transcriptional Effector (LITE) that direct changes in transcriptional activity in a sequence-specific manner. The components of a light inducible system may include an engineered polynucleotide of the present disclosure a light-responsive cytochrome heterodimer (e.g. from Arabidopsis thaliana), and a transcriptional activation/repression domain. In some embodiments, the vector can include one or more of the inducible DNA binding proteins provided in International Patent Publication No. WO 2014/018423 and US Patent Publication Nos., 2015/0291966, 2017/0166903, 2019/0203212, which describe e.g., embodiments of inducible DNA binding proteins and methods of use and can be adapted for use with the present invention.

In some embodiments, transient or inducible expression can be achieved by including, for example, chemical-regulated promotors, i.e., whereby the application of an exogenous chemical induces gene expression. Modulation of gene expression can also be obtained by including a chemical-repressible promoter, where application of the chemical represses gene expression. Chemical-inducible promoters include, but are not limited to, the maize ln2-2 promoter, activated by benzene sulfonamide herbicide safeners (De Veylder et al., (1997) Plant Cell Physiol 38:568-77), the maize GST promoter (GST-11-27, WO93/01294), activated by hydrophobic electrophilic compounds used as pre-emergent herbicides, and the tobacco PR-1 a promoter (Ono et al., (2004) Biosci Biotechnol Biochem 68:803-7) activated by salicylic acid. Promoters which are regulated by antibiotics, such as tetracycline-inducible and tetracycline-repressible promoters (Gatz et al., (1991) Mol Gen Genet 227:229-37; U.S. Pat. Nos. 5,814,618 and 5,789,156) can also be used herein.

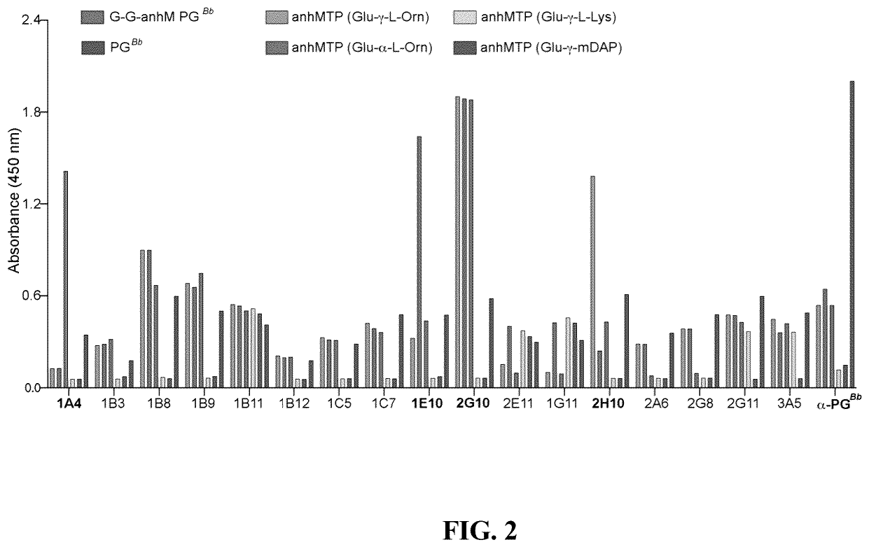

In some embodiments, the polynucleotide, vector or system thereof can include one or more elements capable of translocating and/or expressing an engineered polynucleotide to/in a specific cell component or organelle. Such organelles can include, but are not limited to, nucleus, ribosome, endoplasmic reticulum, Golgi apparatus, chloroplast, mitochondria, vacuole, lysosome, cytoskeleton, plasma membrane, cell wall, peroxisome, centrioles, etc. Such regulatory elements can include, but are not limited to, nuclear localization signals (examples of which are described in greater detail elsewhere herein), any such as those that are annotated in the LocSigDB database (see e.g., http://genome.unmc.edu/LocSigDB/and Negi et al., 2015. Database. 2015: bav003; doi: 10.1093/database/bav003), nuclear export signals (e.g., LXXXLXXLXL (SEQ ID NO: 17) and others described elsewhere herein), endoplasmic reticulum localization/retention signals (e.g., KDEL (SEQ ID NO: 18), KDXX, KKXX, KXX, and others described elsewhere herein; and see e.g., Liu et al. 2007 Mol. Biol. Cell. 18(3): 1073-1082 and Gorleku et al., 2011. J. Biol. Chem. 286:39573-39584), mitochondria (see e.g., Cell Reports. 22:2818-2826, particularly at FIG. 2; Doyle et al. 2013. PLOS ONE 8, e67938; Funes et al. 2002. J. Biol. Chem. 277:6051-6058; Matouschek et al. 1997. PNAS USA 85:2091-2095; Oca-Cossio et al., 2003. 165:707-720; Waltner et al., 1996. J. Biol. Chem. 271:21226-21230; Wilcox et al., 2005. PNAS USA 102:15435-15440; Galanis et al., 1991. FEBS Lett 282:425-430, peroxisome (e.g. (S/A/C)-(K/R/H)-(L/A), SLK, (R/K)-(L/V/I)-XXXXX-(H/Q)-(L/A/F). Suitable protein targeting motifs can also be designed or identified using any suitable database or prediction tool, including but not limited to Minimotif Miner (http: minimotifminer.org, http://mitominer.mrc-mbu.cam.ac.uk/release-4.0/embodiment.do?name=Protein % 20MTS), LocDB (see above), PTSs predictor( ), TargetP-2.0 (http://www.cbs.dtu.dk/services/TargetP/), ChloroP (http://www.cbs.dtu.dk/services/ChloroP/); NetNES (http://www.cbs.dtu.dk/services/NetNES/), Predotar (https://urgi.versailles.inra.fr/predotar/), and SignalP (http://www.cbs.dtu.dk/services/SignalP/).

### Selectable Markers and Tags

One or more of the engineered polynucleotides can be operably linked, fused to, or otherwise modified to include a polynucleotide that encodes or is a selectable marker or tag, which can be a polynucleotide or polypeptide. In some embodiments, the polypeptide encoding a polypeptide selectable marker can be incorporated in the engineered polynucleotide such that the selectable marker polypeptide, when translated, is inserted between two amino acids between the N- and C-terminus of the antibody or fragment thereof of the present disclosure or at the N- and/or C-terminus of the antibody or fragment thereof of the present disclosure. In some embodiments, the selectable marker or tag is a polynucleotide barcode or unique molecular identifier (UMI).

It will be appreciated that the polynucleotide encoding such selectable markers or tags can be incorporated into a polynucleotide encoding one or more components of the antibody or fragment thereof of the present disclosure in an appropriate manner to allow expression of the selectable marker or tag. Such techniques and methods are described elsewhere herein and will be instantly appreciated by one of ordinary skill in the art in view of this disclosure. Many such selectable markers and tags are generally known in the art and are intended to be within the scope of this disclosure.

Suitable selectable markers and tags include, but are not limited to, affinity tags, such as chitin binding protein (CBP), maltose binding protein (MBP), glutathione-S-transferase (GST), poly (His) tag; solubilization tags such as thioredoxin (TRX) and poly(NANP), MBP, and GST; chromatography tags such as those consisting of polyanionic amino acids, such as FLAG-tag; epitope tags such as V5-tag, Myc-tag, HA-tag and NE-tag; protein tags that can allow specific enzymatic modification (such as biotinylation by biotin ligase) or chemical modification (such as reaction with FLASH-EDT2 for fluorescence imaging), DNA and/or RNA segments that contain restriction enzyme or other enzyme cleavage sites; DNA segments that encode products that provide resistance against otherwise toxic compounds including antibiotics, such as, spectinomycin, ampicillin, kanamycin, tetracycline, Basta, neomycin phosphotransferase II (NEO), hygromycin phosphotransferase (HPT)) and the like; DNA and/or RNA segments that encode products that are otherwise lacking in the recipient cell (e.g., tRNA genes, auxotrophic markers); DNA and/or RNA segments that encode products which can be readily identified (e.g., phenotypic markers such as β-galactosidase, GUS; fluorescent proteins such as green fluorescent protein (GFP), cyan (CFP), yellow (YFP), red (RFP), luciferase, and cell surface proteins); polynucleotides that can generate one or more new primer sites for PCR (e.g., the juxtaposition of two DNA sequences not previously juxtaposed), DNA sequences not acted upon or acted upon by a restriction endonuclease or other DNA modifying enzyme, chemical, etc.; epitope tags (e.g. GFP, FLAG- and His-tags), and, DNA sequences that make a molecular barcode or unique molecular identifier (UMI), DNA sequences required for a specific modification (e.g., methylation) that allows its identification. Other suitable markers will be appreciated by those of skill in the art.

Selectable markers and tags can be operably linked to one or more components of the CRISPR-Cas system described herein via suitable linker, such as a glycine or glycine serine linkers as short as GS or GG up to (GGGGG) 3 (SEQ ID NO: 19) or (GGGGS) 3 (SEQ ID NO: 20). Other suitable linkers are described elsewhere herein.

The vector or vector system can include one or more polynucleotides encoding one or more targeting moieties. In some embodiments, the targeting moiety encoding polynucleotides can be included in the vector or vector system, such as a viral vector system, such that they are expressed within and/or on the virus particle(s) produced such that the virus particles can be targeted to specific cells, tissues, organs, etc. In some embodiments, the targeting moiety encoding polynucleotides can be included in the vector or vector system such that the engineered polynucleotide(s) and/or products expressed therefrom include the targeting moiety and can be targeted to specific cells, tissues, organs, etc. In some embodiments, such as non-viral carriers, the targeting moiety can be attached to the carrier (e.g., polymer, lipid, inorganic molecule etc.) and can be capable of targeting the carrier and any attached or associated engineered polynucleotide(s) and/or antibodies or fragments thereof of the present disclosure to specific cells, tissues, organs, etc.

### Codon Optimization of Polynucleotides and Vector Polynucleotides

As described elsewhere herein, the engineered polynucleotide(s) encoding one or more antibodies or fragments thereof of the present disclosure can be codon optimized. In some embodiments, one or more polynucleotides contained in a vector (“vector polynucleotides”) described herein that are in addition to an optionally codon optimized engineered polynucleotide encoding an antibody or fragment thereof of the present disclosure can be codon optimized. In general, codon optimization refers to a process of modifying a nucleic acid sequence for enhanced expression in the host cells of interest by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www.kazusa.orjp/codon/and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, PA), are also available. In some embodiments, one or more codons (e.g., 1, 2, 3, 4, 5, 10, 15, 20, 25, 50, or more, or all codons) in a sequence encoding a DNA/RNA-targeting Cas protein corresponds to the most frequently used codon for a particular amino acid. As to codon usage in yeast, reference is made to the online Yeast Genome database available at http://www.yeastgenome.org/community/codon_usage.shtml, or Codon selection in yeast, Bennetzen and Hall, J Biol Chem. 1982 Mar. 25; 257(6): 3026-31. As to codon usage in plants including algae, reference is made to Codon usage in higher plants, green algae, and cyanobacteria, Campbell and Gowri, Plant Physiol. 1990 January; 92(1): 1-11.; as well as Codon usage in plant genes, Murray et al, Nucleic Acids Res. 1989 Jan. 25; 17(2): 477-98; or Selection on the codon bias of chloroplast and cyanelle genes in different plant and algal lineages, Morton B R, J Mol Evol. 1998 April; 46(4): 449-59.

The vector polynucleotide can be codon optimized for expression in a specific cell-type, tissue type, organ type, and/or subject type. In some embodiments, a codon optimized sequence is a sequence optimized for expression in a eukaryote, e.g., humans (i.e., being optimized for expression in a human or human cell), or for another eukaryote, such as another animal (e.g., a mammal or avian) as is described elsewhere herein. Such codon optimized sequences are within the ambit of the ordinary skilled artisan in view of the description herein. In some embodiments, the polynucleotide is codon optimized for a specific cell type. Such cell types can include, but are not limited to, epithelial cells (including skin cells, cells lining the gastrointestinal tract, cells lining other hollow organs), nerve cells (nerves, brain cells, spinal column cells, nerve support cells (e.g., astrocytes, glial cells, Schwann cells etc.), muscle cells (e.g., cardiac muscle, smooth muscle cells, and skeletal muscle cells), connective tissue cells (fat and other soft tissue padding cells, bone cells, tendon cells, cartilage cells), blood cells, stem cells and other progenitor cells, immune system cells, germ cells, and combinations thereof. Such codon optimized sequences are within the ambit of the ordinary skilled artisan in view of the description herein. In some embodiments, the polynucleotide is codon optimized for a specific tissue type. Such tissue types can include, but are not limited to, muscle tissue, connective tissue, connective tissue, nervous tissue, and epithelial tissue. Such codon optimized sequences are within the ambit of the ordinary skilled artisan in view of the description herein. In some embodiments, the polynucleotide is codon optimized for a specific organ. Such organs include, but are not limited to, muscles, skin, intestines, liver, spleen, brain, lungs, stomach, heart, kidneys, gallbladder, pancreas, bladder, thyroid, bone, blood vessels, blood, and combinations thereof. Such codon optimized sequences are within the ambit of the ordinary skilled artisan in view of the description herein.

In some embodiments, a vector polynucleotide is codon optimized for expression in particular cells, such as prokaryotic or eukaryotic cells. The eukaryotic cells may be those of or derived from a particular organism, such as a plant or a mammal, including but not limited to human, or non-human eukaryote or animal or mammal as discussed herein, e.g., mouse, rat, rabbit, dog, livestock, or non-human mammal or primate.

### Vector Construction

The vectors described herein can be constructed using any suitable process or technique. In some embodiments, one or more suitable recombination and/or cloning methods or techniques can be used to the vector(s) described herein. Suitable recombination and/or cloning techniques and/or methods can include, but not limited to, those described in U.S. Patent Publication No. US 2004/0171156 A1. Other suitable methods and techniques are described elsewhere herein.

Construction of recombinant AAV vectors are described in a number of publications, including U.S. Pat. No. 5,173,414; Tratschin et al., Mol. Cell. Biol. 5:3251-3260(1985); Tratschin, et al., Mol. Cell. Biol. 4:2072-2081(1984); Hermonat & Muzyczka, PNAS 81:6466-6470(1984); and Samulski et al., J. Virol. 63:03822-3828(1989). Any of the techniques and/or methods can be used and/or adapted for constructing an AAV or other vector described herein. nAAV vectors are discussed elsewhere herein.

In some embodiments, a vector comprises one or more insertion sites, such as a restriction endonuclease recognition sequence (also referred to as a “cloning site”). In some embodiments, one or more insertion sites (e.g., about or more than about 1, 2, 3, 4, 5, 6, 7, 8, 9, 10, or more insertion sites) are located upstream and/or downstream of one or more sequence elements of one or more vectors. When multiple different guide polynucleotides are used, a single expression construct may be used to target nucleic acid-targeting activity to multiple different, corresponding target sequences within a cell. For example, a single vector may comprise about or more than about 1, 2, 3, 4, 5, 6, 7, 8, 9, 10, 15, 20, or more guide s polynucleotides. In some embodiments, about or more than about 1, 2, 3, 4, 5, 6, 7, 8, 9, 10, or more such guide-polynucleotide-containing vectors may be provided, and optionally delivered to a cell.

### Viral Vectors

In some embodiments, the vector is a viral vector. The term of art “viral vector” and as used herein in this context refers to polynucleotide-based vectors that contain one or more elements from or based upon one or more elements of a virus that can be capable of expressing and packaging a polynucleotide, such as an engineered polynucleotide of the present invention, into a virus particle and producing said virus particle when used alone or with one or more other viral vectors (such as in a viral vector system). Viral vectors and systems thereof can be used for producing viral particles for delivery of and/or expression of one or more antibodies or fragments thereof described herein. The viral vector can be part of a viral vector system involving multiple vectors. In some embodiments, systems incorporating multiple viral vectors can increase the safety of these systems. Suitable viral vectors can include retroviral-based vectors, lentiviral-based vectors, adenoviral-based vectors, adeno associated vectors, helper-dependent adenoviral (HdAd) vectors, hybrid adenoviral vectors, herpes simplex virus-based vectors, poxvirus-based vectors, and Epstein-Barr virus-based vectors. Other embodiments of viral vectors and viral particles produce therefrom are described elsewhere herein. In some embodiments, the viral vectors are configured to produce replication incompetent viral particles for improved safety of these systems.

The systems and compositions of the present disclosure may be delivered to plant cells using viral vehicles. In particular embodiments, the compositions and systems may be introduced in the plant cells using a plant viral vector (e.g., as described in Scholthof et al. 1996, Annu Rev Phytopathol. 1996; 34:299-323). Such viral vector may be a vector from a DNA virus, e.g., geminivirus (e.g., cabbage leaf curl virus, bean yellow dwarf virus, wheat dwarf virus, tomato leaf curl virus, maize streak virus, tobacco leaf curl virus, or tomato golden mosaic virus) or nanovirus (e.g., Faba bean necrotic yellow virus). The viral vector may be a vector from an RNA virus, e.g., tobravirus (e.g., tobacco rattle virus, tobacco mosaic virus), potexvirus (e.g., potato virus X), or hordeivirus (e.g., barley stripe mosaic virus). The replicating genomes of plant viruses may be non-integrative vectors.

Methods and techniques for virus production from viral vectors are generally known in the art.

### Non-Viral Vectors

In some embodiments, the vector is a non-viral vector or vector system. The term of art “Non-viral vector” and as used herein in this context refers to molecules and/or compositions that are vectors but that are not based on one or more component of a virus or virus genome (excluding any nucleotide to be delivered and/or expressed by the non-viral vector) that can be capable of incorporating the engineered polynucleotide(s) and delivering said engineered polynucleotide(s) to a cell and/or expressing the polynucleotide in the cell. It will be appreciated that this does not exclude vectors containing a polynucleotide designed to target a virus-based polynucleotide that is to be delivered.

### Naked Polynucleotides

In some embodiments one or more engineered polynucleotides described elsewhere herein can be included in a naked polynucleotide. The term of art “naked polynucleotide” as used herein refers to polynucleotides that are not associated with another molecule (e.g., proteins, lipids, and/or other molecules) that can often help protect it from environmental factors and/or degradation. As used herein, associated with includes, but is not limited to, linked to, adhered to, adsorbed to, enclosed in, enclosed in or within, mixed with, and the like. Naked polynucleotides that include one or more of the engineered polynucleotides described herein can be delivered directly to a host cell and optionally expressed therein. The naked polynucleotides can have any suitable two- and three-dimensional configurations. By way of non-limiting examples, naked polynucleotides can be single-stranded molecules, double stranded molecules, circular molecules (e.g., plasmids and artificial chromosomes), molecules that contain portions that are single stranded and portions that are double stranded (e.g., ribozymes), and the like. In some embodiments, the naked polynucleotide contains only the engineered polynucleotide(s) of the present invention. In some embodiments, the naked polynucleotide can contain other nucleic acids and/or polynucleotides in addition to the engineered polynucleotide(s) of the present invention. The naked polynucleotides can include one or more elements of a transposon system. Transposons and systems thereof are described in greater detail elsewhere herein.

### Non-Viral Polynucleotide Vectors

In some embodiments, one or more of the engineered polynucleotides can be included in a non-viral polynucleotide vector. Suitable non-viral polynucleotide vectors include, but are not limited to, transposon vectors and vector systems, plasmids, bacterial artificial chromosomes, yeast artificial chromosomes, AR (antibiotic resistance)-free plasmids and miniplasmids, circular covalently closed vectors (e.g. minicircles, minivectors, miniknots), linear covalently closed vectors (“dumbbell shaped”), MIDGE (minimalistic immunologically defined gene expression) vectors, MiLV (micro-linear vector) vectors, Ministrings, mini-intronic plasmids, PSK systems (post-segregationally killing systems), ORT (operator repressor titration) plasmids, and the like. See e.g., Hardee et al. 2017. Genes. 8(2): 65.

In some embodiments, the non-viral polynucleotide vector can have a conditional origin of replication. In some embodiments, the non-viral polynucleotide vector can be an ORT plasmid. In some embodiments, the non-viral polynucleotide vector can have a minimalistic immunologically defined gene expression. In some embodiments, the non-viral polynucleotide vector can have one or more post-segregationally killing system genes. In some embodiments, the non-viral polynucleotide vector is AR-free. In some embodiments, the non-viral polynucleotide vector is a minivector. In some embodiments, the non-viral polynucleotide vector includes a nuclear localization signal. In some embodiments, the non-viral polynucleotide vector can include one or more CpG motifs. In some embodiments, the non-viral polynucleotide vectors can include one or more scaffold/matrix attachment regions (S/MARs). See e.g., Mirkovitch et al. 1984. Cell. 39:223-232, Wong et al. 2015. Adv. Genet. 89:113-152, whose techniques and vectors can be adapted for use in the present invention. S/MARs are AT-rich sequences that play a role in the spatial organization of chromosomes through DNA loop base attachment to the nuclear matrix. S/MARs are often found close to regulatory elements such as promoters, enhancers, and origins of DNA replication. Inclusion of one or S/MARs can facilitate a once-per-cell-cycle replication to maintain the non-viral polynucleotide vector as an episome in daughter cells. In certain embodiments, the S/MAR sequence is located downstream of an actively transcribed polynucleotide (e.g., one or more CRISPR-Cas system polynucleotides of the present invention) included in the non-viral polynucleotide vector. In some embodiments, the S/MAR can be a S/MAR from the beta-interferon gene cluster. See e.g., Verghese et al. 2014. Nucleic Acid Res. 42: e53; Xu et al. 2016. Sci. China Life Sci. 59:1024-1033; Jin et al. 2016. 8:702-711; Koirala et al. 2014. Adv. Exp. Med. Biol. 801:703-709; and Nehlsen et al. 2006. Gene Ther. Mol. Biol. 10:233-244, whose techniques and vectors can be adapted for use in the present invention.

In some embodiments, the non-viral vector is a transposon vector or system thereof. As used herein, “transposon” (also referred to as transposable element) refers to a polynucleotide sequence that is capable of moving form location in a genome to another. There are several classes of transposons. Transposons include retrotransposons and DNA transposons. Retrotransposons require the transcription of the polynucleotide that is moved (or transposed) in order to transpose the polynucleotide to a new genome or polynucleotide. DNA transposons are those that do not require reverse transcription of the polynucleotide that is moved (or transposed) in order to transpose the polynucleotide to a new genome or polynucleotide. In some embodiments, the non-viral polynucleotide vector can be a retrotransposon vector. In some embodiments, the retrotransposon vector includes long terminal repeats. In some embodiments, the retrotransposon vector does not include long terminal repeats. In some embodiments, the non-viral polynucleotide vector can be a DNA transposon vector. DNA transposon vectors can include a polynucleotide sequence encoding a transposase. In some embodiments, the transposon vector is configured as a non-autonomous transposon vector, meaning that the transposition does not occur spontaneously on its own. In some of these embodiments, the transposon vector lacks one or more polynucleotide sequences encoding proteins required for transposition. In some embodiments, the non-autonomous transposon vectors lack one or more Ac elements.

In some embodiments a non-viral polynucleotide transposon vector system can include a first polynucleotide vector that contains the engineered polynucleotide(s) of the present invention flanked on the 5′ and 3′ ends by transposon terminal inverted repeats (TIRs) and a second polynucleotide vector that includes a polynucleotide capable of encoding a transposase coupled to a promoter to drive expression of the transposase. When both are expressed in the same cell the transposase can be expressed from the second vector and can transpose the material between the TIRs on the first vector (e.g., the engineered polynucleotide(s) of the present invention) and integrate it into one or more positions in the host cell's genome. In some embodiments the transposon vector or system thereof can be configured as a gene trap. In some embodiments, the TIRs can be configured to flank a strong splice acceptor site followed by a reporter and/or other gene (e.g., one or more of the engineered polynucleotide(s) of the present invention) and a strong poly A tail. When transposition occurs while using this vector or system thereof, the transposon can insert into an intron of a gene and the inserted reporter or other gene can provoke a mis-splicing process and as a result it in activates the trapped gene.

Any suitable transposon system can be used. Suitable transposon and systems thereof can include, without limitation, Sleeping Beauty transposon system (Tc1/mariner superfamily) (see e.g., Ivics et al. 1997. Cell. 91(4): 501-510), piggyBac (piggyBac superfamily) (see e.g., Li et al. 2013 110(25): E2279-E2287 and Yusa et al. 2011. PNAS. 108(4): 1531-1536), Tol2 (superfamily hAT), Frog Prince (Tc1/mariner superfamily) (see e.g., Miskey et al. 2003 Nucleic Acid Res. 31(23): 6873-6881) and variants thereof.

### Antibody Production

The anti-peptidoglycan antibodies and fragments thereof disclosed herein can be produced by any method known in the art for the synthesis of antibodies, for example, by chemical synthesis or by recombinant expression techniques. The methods described herein employ, unless otherwise indicated, conventional techniques in molecular biology, microbiology, genetic analysis, recombinant DNA, organic chemistry, biochemistry, PCR, oligonucleotide synthesis and modification, nucleic acid hybridization, and related fields within the skill of the art. These techniques are described, for example, in the references cited herein and are fully explained in the literature. See, e.g., Maniatis T et al., (1982) Molecular Cloning: A Laboratory Manual, Cold Spring Harbor Laboratory Press; Sambrook J et al., (1989), Molecular Cloning: A Laboratory Manual, Second Edition, Cold Spring Harbor Laboratory Press; Sambrook J et al., (2001) Molecular Cloning: A Laboratory Manual, Cold Spring Harbor Laboratory Press, Cold Spring Harbor, NY; Ausubel F M et al., Current Protocols in Molecular Biology, John Wiley & Sons (1987 and annual updates); Current Protocols in Immunology, John Wiley & Sons (1987 and annual updates) Gait (ed.) (1984) Oligonucleotide Synthesis: A Practical Approach, IRL Press; Eckstein (ed.) (1991) Oligonucleotides and Analogues: A Practical Approach, IRL Press; Birren B et al., (eds.) (1999) Genome Analysis: A Laboratory Manual, Cold Spring Harbor Laboratory Press.

In some embodiments, an antibody or fragment thereof described herein is an antibody (e.g., recombinant antibody) prepared, expressed, created, or isolated by any means that involves creation, e.g., via synthesis, genetic engineering of DNA sequences. In certain embodiments, such antibody or fragment thereof comprises sequences (e.g., DNA sequences or amino acid sequences) that do not naturally exist within the antibody germline repertoire of an animal or mammal (e.g., human) in vivo.

### Pharmaceutical Formulations

Also described herein are pharmaceutical formulations that can contain an amount, effective amount, and/or least effective amount, and/or therapeutically effective amount of one or more compounds, molecules, compositions, vectors, vector systems, cells, or a combination thereof (which are also referred to as the primary active agent or ingredient elsewhere herein) described in greater detail elsewhere herein and a pharmaceutically acceptable carrier or excipient. In some embodiments, the pharmaceutical formulation comprises an antibody of fragment thereof of the present disclosure as a primary or secondary active agent. As used herein, “pharmaceutical formulation” refers to the combination of an active agent, compound, or ingredient with a pharmaceutically acceptable carrier or excipient, making the composition suitable for diagnostic, therapeutic, or preventive use in vitro, in vivo, or ex vivo. As used herein, “pharmaceutically acceptable carrier or excipient” refers to a carrier or excipient that is useful in preparing a pharmaceutical formulation that is generally safe, non-toxic, and is neither biologically or otherwise undesirable, and includes a carrier or excipient that is acceptable for veterinary use as well as human pharmaceutical use. A “pharmaceutically acceptable carrier or excipient” as used in the specification and claims includes both one and more than one such carrier or excipient. When present, the compound can optionally be present in the pharmaceutical formulation as a pharmaceutically acceptable salt.

In some embodiments, the active ingredient is present as a pharmaceutically acceptable salt of the active ingredient. As used herein, “pharmaceutically acceptable salt” refers to any acid or base addition salt whose counter-ions are non-toxic to the subject to which they are administered in pharmaceutical doses of the salts. Suitable salts include, hydrobromide, iodide, nitrate, bisulfate, phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, napthalenesulfonate, propionate, malonate, mandelate, malate, phthalate, and pamoate.

The pharmaceutical formulations described herein can be administered to a subject in need thereof via any suitable method or route to a subject in need thereof. Suitable administration routes can include, but are not limited to auricular (otic), buccal, conjunctival, cutaneous, dental, electro-osmosis, endocervical, endosinusial, endotracheal, enteral, epidural, extra-amniotic, extracorporeal, hemodialysis, infiltration, interstitial, intra-abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal (dental), intracoronary, intracorporus cavernosum, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratympanic, intrauterine, intravascular, intravenous, intravenous bolus, intravenous drip, intraventricular, intravesical, intravitreal, iontophoresis, irrigation, laryngeal, nasal, nasogastric, occlusive dressing technique, ophthalmic, oral, oropharyngeal, other, parenteral, percutaneous, periarticular, peridural, perineural, periodontal, rectal, respiratory (inhalation), retrobulbar, soft tissue, subarachnoid, subconjunctival, subcutaneous, sublingual, submucosal, topical, transdermal, transmucosal, transplacental, transtracheal, transtympanic, ureteral, urethral, and/or vaginal administration, and/or any combination of the above administration routes, which typically depends on the disease to be treated and/or the active ingredient(s).

Where appropriate, compounds, molecules, compositions, vectors, vector systems, cells, or any combination thereof described in greater detail elsewhere herein can be provided to a subject in need thereof as an ingredient, such as an active ingredient or agent, in a pharmaceutical formulation. As such, also described are pharmaceutical formulations containing one or more of the compounds and salts thereof, or pharmaceutically acceptable salts thereof described herein. Suitable salts include, hydrobromide, iodide, nitrate, bisulfate, phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, napthalenesulfonate, propionate, malonate, mandelate, malate, phthalate, and pamoate.

In some embodiments, the subject in need thereof has or is suspected of having a Lyme disease and/or B. Burgdorferi infection, or a symptom thereof. As used herein, “agent” refers to any substance, compound, molecule, and the like, which can be biologically active or otherwise can induce a biological and/or physiological effect on a subject to which it is administered to. As used herein, “active agent” or “active ingredient” refers to a substance, compound, or molecule, which is biologically active or otherwise, induces a biological or physiological effect on a subject to which it is administered to. In other words, “active agent” or “active ingredient” refers to a component or components of a composition to which the whole or part of the effect of the composition is attributed. An agent can be a primary active agent, or in other words, the component(s) of a composition to which the whole or part of the effect of the composition is attributed. An agent can be a secondary agent, or in other words, the component(s) of a composition to which an additional part and/or other effect of the composition is attributed.

### Pharmaceutically Acceptable Carriers and Secondary Ingredients and Agents

The pharmaceutical formulation can include a pharmaceutically acceptable carrier. Suitable pharmaceutically acceptable carriers include, but are not limited to water, salt solutions, alcohols, gum arabic, vegetable oils, benzyl alcohols, polyethylene glycols, gelatin, carbohydrates such as lactose, amylose or starch, magnesium stearate, talc, silicic acid, viscous paraffin, perfume oil, fatty acid esters, hydroxy methylcellulose, and polyvinyl pyrrolidone, which do not deleteriously react with the active composition.

The pharmaceutical formulations can be sterilized, and if desired, mixed with agents, such as lubricants, preservatives, stabilizers, wetting agents, emulsifiers, salts for influencing osmotic pressure, buffers, coloring, flavoring and/or aromatic substances, and the like which do not deleteriously react with the active compound.

In some embodiments, the pharmaceutical formulation can also include an effective amount of secondary active agents, including but not limited to, biologic agents or molecules including, but not limited to, e.g., polynucleotides, amino acids, peptides, polypeptides, antibodies, aptamers, ribozymes, hormones, immunomodulators, antipyretics, anxiolytics, antipsychotics, analgesics, antispasmodics, anti-inflammatories, anti-histamines, anti-infectives, chemotherapeutics, and any combinations thereof.

In some embodiments, the secondary active agent can include one or more agents conventionally used to treat Lyme disease or a symptom or secondary condition thereof.

In some embodiments, the secondary active agent is or includes, but is not limited to, polynucleotides, amino acids, peptides, polypeptides, antibodies, aptamers, ribozymes, hormones, immunomodulators, antipyretics, anxiolytics, antipsychotics, analgesics, antispasmodics, anti-inflammatories, anti-histamines, anti-infectives, hydrolases, chemotherapeutics, and combinations thereof.

In some embodiments, the secondary active agent is or includes an anti-infective.

In certain embodiments, the secondary active agent is or includes a peptidoglycan-specific hydrolase.

In certain embodiments, the secondary active agent is or includes an analgesic.

In certain embodiments, the secondary active agent is or includes an anti-pyretic.

In some embodiments, the secondary active agent comprises an anti-infective and a peptidoglycan-specific hydrolase.

In some embodiments, the secondary active agent comprises, a peptidoglycan-specific hydrolase, an anti-infective, and an analgesic.

In some embodiments, the secondary active agent comprises, a peptidoglycan-specific hydrolase, an anti-infective, an analgesic, and an anti-pyretic.

In some embodiments, the anti-infective agent is or includes doxycycline, amoxicillin, cefuroxime, cefotaxime, azlocillin, penicillin, erythromycin, ceftriaxone, or any active derivative or analogue thereof, or any combination thereof.

Exemplary peptidoglycan-specific hydrolases include an amidase or a lysozyme.

Exemplary antipyretics include, but are not limited to, non-steroidal anti-inflammatories (e.g., ibuprofen, naproxen, ketoprofen, and nimesulide), aspirin and related salicylates (e.g., choline salicylate, magnesium salicylate, and sodium salicylate), paracetamol/acetaminophen, metamizole, nabumetone, phenazone, and quinine.

Exemplary analgesics include, but are not limited to, paracetamol/acetaminophen, nonsteroidal anti-inflammantories (e.g., ibuprofen, naproxen, ketoprofen, and nimesulide), COX-2 inhibitors (e.g., rofecoxib, celecoxib, and etoricoxib), opioids (e.g. morphine, codeine, oxycodone, hydrocodone, dihydromorphine, pethidine, buprenorphine), tramadol, norepinephrine, flupirtine, nefopam, orphenadrine, pregabalin, gabapentin, cyclobenzaprine, scopolamine, methadone, ketobemidone, piritramide, and aspirin and related salicylates (e.g., choline salicylate, magnesium salicylate, and sodium salicylate).

As used herein, “anti-infective” refers to compounds or molecules that can either kill an infectious agent and/or modulate or inhibit its activity, infectivity, replication, and/or spreading such that its infectivity is reduced or eliminated and/or the disease or symptom thereof that it is associated is less severe or eliminated. Anti-infectives include, but are not limited to, antibiotics, antibacterials, antifungals, antivirals, and antiprotozoals. Suitable anti-infectives include, but are not limited to, amebicides (e.g., nitazoxanide, paromomycin, metronidazole, tinidazole, chloroquine, miltefosine, amphotericin b, and iodoquinol), aminoglycosides (e.g., paromomycin, tobramycin, gentamicin, amikacin, kanamycin, and neomycin), anthelmintics (e.g., pyrantel, mebendazole, ivermectin, praziquantel, albendazole, thiabendazole, oxamniquine), antifungals (e.g., azole antifungals (e.g., itraconazole, fluconazole, posaconazole, ketoconazole, clotrimazole, miconazole, and voriconazole), echinocandins (e.g., caspofungin, anidulafungin, and micafungin), griseofulvin, terbinafine, flucytosine, and polyenes (e.g., nystatin, and amphotericin b), antimalarial agents (e.g., pyrimethamine/sulfadoxine, artemether/lumefantrine, atovaquone/proquanil, quinine, hydroxychloroquine, mefloquine, chloroquine, doxycycline, pyrimethamine, and halofantrine), antituberculosis agents (e.g., aminosalicylates (e.g., amino salicylic acid), isoniazid/rifampin, isoniazid/pyrazinamide/rifampin, bedaquiline, isoniazid, ethambutol, rifampin, rifabutin, rifapentine, capreomycin, and cycloserine), antivirals (e.g., amantadine, rimantadine, abacavir/lamivudine, emtricitabine/tenofovir, cobicistat/elvitegravir/emtricitabine/tenofovir, efavirenz/emtricitabine/tenofovir, abacavir/lamivudine/zidovudine, lamivudine/zidovudine, emtricitabine/tenofovir, emtricitabine/lopinavir/ritonavir/tenofovir, interferon alfa-2v/ribavirin, peginterferon alfa-2b, maraviroc, raltegravir, dolutegravir, enfuvirtide, foscarnet, fomivirsen, oseltamivir, zanamivir, nevirapine, efavirenz, etravirine, rilpivirine, delavirdine, nevirapine, entecavir, lamivudine, adefovir, sofosbuvir, didanosine, tenofovir, abacavir, zidovudine, stavudine, emtricitabine, zalcitabine, telbivudine, simeprevir, boceprevir, telaprevir, lopinavir/ritonavir, fosamprenavir, darunavir, ritonavir, tipranavir, atazanavir, nelfinavir, amprenavir, indinavir, saquinavir, ribavirin, valacyclovir, acyclovir, famciclovir, ganciclovir, and valganciclovir), carbapenems (e.g., doripenem, meropenem, ertapenem, and cilastatin/imipenem), cephalosporins (e.g., cefadroxil, cephradine, cefazolin, cephalexin, cefepime, ceflaroline, loracarbef, cefotetan, cefuroxime, cefprozil, loracarbef, cefoxitin, cefaclor, ceftibuten, ceftriaxone, cefotaxime, cefpodoxime, cefdinir, cefixime, cefditoren, cefizoxime, and ceftazidime), glycopeptide antibiotics (e.g., vancomycin, dalbavancin, oritavancin, and telavancin), glycylcyclines (e.g., tigecycline), leprostatics (e.g., clofazimine and thalidomide), lincomycin and derivatives thereof (e.g., clindamycin and lincomycin), macrolides and derivatives thereof (e.g., telithromycin, fidaxomicin, erythromycin, azithromycin, clarithromycin, dirithromycin, and troleandomycin), linezolid, sulfamethoxazole/trimethoprim, rifaximin, chloramphenicol, fosfomycin, metronidazole, aztreonam, bacitracin, penicillins (amoxicillin, ampicillin, bacampicillin, carbenicillin, piperacillin, ticarcillin, amoxicillin/clavulanate, ampicillin/sulbactam, piperacillin/tazobactam, clavulanate/ticarcillin, penicillin, procaine penicillin, oxacillin, dicloxacillin, and nafcillin), quinolones (e.g., lomefloxacin, norfloxacin, ofloxacin, moxifloxacin, ciprofloxacin, levofloxacin, Gemifloxacin, moxifloxacin, cinoxacin, nalidixic acid, enoxacin, grepafloxacin, gatifloxacin, trovafloxacin, and sparfloxacin), sulfonamides (e.g., sulfamethoxazole/trimethoprim, sulfasalazine, and sulfasoxazole), tetracyclines (e.g., doxycycline, demeclocycline, minocycline, doxycycline/salicylic acid, doxycycline/omega-3 polyunsaturated fatty acids, and tetracycline), and urinary anti-infectives (e.g., nitrofurantoin, methenamine, fosfomycin, cinoxacin, nalidixic acid, trimethoprim, and methylene blue).

### Effective Amounts

In some embodiments, the amount of the primary active agent and/or optional secondary agent can be an effective amount, least effective amount, and/or therapeutically effective amount. As used herein, “effective amount”, “effective concentration”, and/or the like refers to the amount, concentration, etc. of the primary and/or optional secondary agent included in the pharmaceutical formulation that achieve one or more therapeutic effects or desired effect. As used herein, “least effective”, “least effective concentration”, and/or the like amount refers to the lowest amount, concentration, etc. of the primary and/or optional secondary agent that achieves the one or more therapeutic or other desired effects. As used herein, “therapeutically effective amount”, “therapeutically effective concentration” and/or the like refers to the amount, concentration, etc. of the primary and/or optional secondary agent included in the pharmaceutical formulation that achieves one or more therapeutic effects. In some embodiments, the one or more therapeutic effects are to treat or prevent infection with B. Burgdorferi, treat or prevent Lyme disease, or a symptom thereof.

The effective amount, least effective amount, and/or therapeutically effective amount of the primary and optional secondary active agent described elsewhere herein contained in the pharmaceutical formulation can be any non-zero amount ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1,000 μg, ng, μg, mg, or g or be any numerical value or subrange within any of these ranges.

In some embodiments, the effective amount, least effective amount, and/or therapeutically effective amount can be an effective concentration, least effective concentration, and/or therapeutically effective concentration, which can each be any non-zero amount ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1,000 pM, nM, μM, mM, or M or be any numerical value or subrange within any of these ranges.

In other embodiments, the effective amount, least effective amount, and/or therapeutically effective amount of the primary and optional secondary active agent be any non-zero amount ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1,000 IU or be any numerical value or subrange within any of these ranges.

In some embodiments, the primary and/or the optional secondary active agent present in the pharmaceutical formulation can be any non-zero amount ranging from about 0 to 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.9, to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, or 99.9% w/w, v/v, or w/v of the pharmaceutical formulation or be any numerical value or subrange within any of these ranges.

In some embodiments where a cell or cell population is present in the pharmaceutical formulation (e.g., as a primary and/or or secondary active agent), the effective amount of cells can be any amount ranging from about 1 or 2 cells to 1×10¹ cells/mL, 1×10²⁰ cells/mL or more, such as about 1×10¹ cells/mL, 1×10² cells/mL, 1×10³ cells/mL, 1×10⁴ cells/mL, 1×10⁵ cells/mL, 1×10⁶ cells/mL, 1×10⁷ cells/mL, 1×10⁸ cells/mL, 1×10⁹ cells/mL, 1×10¹⁰ cells/mL, 1×10¹¹ cells/mL, 1×10¹² cells/mL, 1×10¹³ cells/mL, 1×10¹⁴ cells/mL, 1×10¹⁵ cells/mL, 1×10¹⁶ cells/mL, 1×10¹⁷ cells/mL, 1×10¹⁸ cells/mL, 1×10¹⁹ cells/mL, to/or about 1×10²⁰ cells/mL, or any numerical value or subrange within any of these ranges.

In some embodiments, the amount or effective amount, particularly where an infective particle is being delivered (e.g., a virus particle having the primary or secondary agent as a cargo), the effective amount of virus particles can be expressed as a titer (plaque forming units per unit of volume) or as a MOI (multiplicity of infection). In some embodiments, the effective amount can be about 1×10¹ particles per pL, nL, μL, mL, or L to 1×10²⁰/particles per pL, nL, μL, mL, or L or more, such as about 1×10¹, 1×10², 1×10³, 1×10⁴, 1×1⁰⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, 1×10¹⁸, 1×10¹⁹, to/or about 1×10²⁰ particles per pL, nL, μL, mL, or L. In some embodiments, the effective titer can be about 1×10¹ transforming units per pL, nL, μL, mL, or L to 1×10²⁰/transforming units per pL, nL, μL, mL, or L or more, such as about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×1¹³, 1×10¹⁴, 1×10¹⁵, 1×10¹⁶, 1×10¹⁷, 1×10¹⁸, 1×10¹⁹, to/or about 1×10²⁰ transforming units per pL, nL, μL, mL, or L or any numerical value or subrange within these ranges. In some embodiments, the MOI of the pharmaceutical formulation can range from about 0.1 to 10 or more, such as 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, or more or any numerical value or subrange within these ranges.

In some embodiments, the amount or effective amount of the one or more of the active agent(s) described herein contained in the pharmaceutical formulation can range from about 1 μg/kg to about 10 mg/kg based upon the bodyweight of the subject in need thereof or average bodyweight of the specific patient population to which the pharmaceutical formulation can be administered.

In embodiments where there is a secondary agent contained in the pharmaceutical formulation, the effective amount of the secondary active agent will vary depending on the secondary agent, the primary agent, the administration route, subject age, disease, stage of disease, among other things, which will be one of ordinary skill in the art.

When optionally present in the pharmaceutical formulation, the secondary active agent can be included in the pharmaceutical formulation or can exist as a stand-alone compound or pharmaceutical formulation that can be administered contemporaneously or sequentially with the compound, derivative thereof, or pharmaceutical formulation thereof.

In some embodiments, the effective amount of the secondary active agent, when optionally present, is any non-zero amount ranging from about 0 to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, or 99.9% w/w, v/v, or w/v of the total active agents present in the pharmaceutical formulation or any numerical value or subrange within these ranges. In additional embodiments, the effective amount of the secondary active agent is any non-zero amount ranging from about 0 to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, or 99.9% w/w, v/v, or w/v of the total pharmaceutical formulation or any numerical value or subrange within these ranges.

### Dosage Forms

In some embodiments, the pharmaceutical formulations described herein can be provided in a dosage form. The dosage form can be administered to a subject in need thereof. The dosage form can be effective generate specific concentration, such as an effective concentration, at a given site in the subject in need thereof. As used herein, “dose,” “unit dose,” or “dosage” can refer to physically discrete units suitable for use in a subject, each unit containing a predetermined quantity of the primary active agent, and optionally present secondary active ingredient, and/or a pharmaceutical formulation thereof calculated to produce the desired response or responses in association with its administration. In some embodiments, the given site is proximal to the administration site. In some embodiments, the given site is distal to the administration site. In some cases, the dosage form contains a greater amount of one or more of the active ingredients present in the pharmaceutical formulation than the final intended amount needed to reach a specific region or location within the subject to account for loss of the active components such as via first and second pass metabolism.

The dosage forms can be adapted for administration by any appropriate route. Appropriate routes include, but are not limited to, oral (including buccal or sublingual), rectal, intraocular, inhaled, intranasal, topical (including buccal, sublingual, or transdermal), vaginal, parenteral, subcutaneous, intramuscular, intravenous, internasal, and intradermal. Other appropriate routes are described elsewhere herein. Such formulations can be prepared by any method known in the art.

Dosage forms adapted for oral administration can discrete dosage units such as capsules, pellets or tablets, powders or granules, solutions, or suspensions in aqueous or non-aqueous liquids; edible foams or whips, or in oil-in-water liquid emulsions or water-in-oil liquid emulsions. In some embodiments, the pharmaceutical formulations adapted for oral administration also include one or more agents which flavor, preserve, color, or help disperse the pharmaceutical formulation. Dosage forms prepared for oral administration can also be in the form of a liquid solution that can be delivered as a foam, spray, or liquid solution. The oral dosage form can be administered to a subject in need thereof. Where appropriate, the dosage forms described herein can be microencapsulated.

The dosage form can also be prepared to prolong or sustain the release of any ingredient. In some embodiments, compounds, molecules, compositions, vectors, vector systems, cells, or a combination thereof described herein can be the ingredient whose release is delayed. In some embodiments the primary active agent is the ingredient whose release is delayed. In some embodiments, an optional secondary agent can be the ingredient whose release is delayed. Suitable methods for delaying the release of an ingredient include, but are not limited to, coating or embedding the ingredients in material in polymers, wax, gels, and the like. Delayed release dosage formulations can be prepared as described in standard references such as “Pharmaceutical dosage form tablets,” eds. Liberman et. al. (New York, Marcel Dekker, Inc., 1989), “Remington—The science and practice of pharmacy”, 20th ed., Lippincott Williams & Wilkins, Baltimore, MD, 2000, and “Pharmaceutical dosage forms and drug delivery systems”, 6th Edition, Ansel et al., (Media, PA: Williams and Wilkins, 1995). These references provide information on excipients, materials, equipment, and processes for preparing tablets and capsules and delayed release dosage forms of tablets and pellets, capsules, and granules. The delayed release can be anywhere from about an hour to about 3 months or more.

Examples of suitable coating materials include, but are not limited to, cellulose polymers such as cellulose acetate phthalate, hydroxypropyl cellulose, hydroxypropyl methylcellulose, hydroxypropyl methylcellulose phthalate, and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Coatings may be formed with a different ratio of water-soluble polymer, water insoluble polymers, and/or pH dependent polymers, with or without water insoluble/water soluble non-polymeric excipient, to produce the desired release profile. The coating is either performed on the dosage form (matrix or simple) which includes, but is not limited to, tablets (compressed with or without coated beads), capsules (with or without coated beads), beads, particle compositions, “ingredient as is” formulated as, but not limited to, suspension form or as a sprinkle dosage form.

Where appropriate, the dosage forms described herein can be a liposome. In these embodiments, primary active ingredient(s), and/or optional secondary active ingredient(s), and/or pharmaceutically acceptable salt thereof where appropriate are incorporated into a liposome. In embodiments where the dosage form is a liposome, the pharmaceutical formulation is thus a liposomal formulation. The liposomal formulation can be administered to a subject in need thereof.

Dosage forms adapted for topical administration can be formulated as ointments, creams, suspensions, lotions, powders, solutions, pastes, gels, sprays, aerosols, or oils. In some embodiments for treatments of the eye or other external tissues, for example the mouth or the skin, the pharmaceutical formulations are applied as a topical ointment or cream. When formulated in an ointment, a primary active ingredient, optional secondary active ingredient, and/or pharmaceutically acceptable salt thereof where appropriate can be formulated with a paraffinic or water-miscible ointment base. In other embodiments, the primary and/or secondary active ingredient can be formulated in a cream with an oil-in-water cream base or a water-in-oil base. Dosage forms adapted for topical administration in the mouth include lozenges, pastilles, and mouth washes.

Dosage forms adapted for nasal or inhalation administration include aerosols, solutions, suspension drops, gels, or dry powders. In some embodiments, a primary active ingredient, optional secondary active ingredient, and/or pharmaceutically acceptable salt thereof where appropriate can be in a dosage form adapted for inhalation is in a particle-size-reduced form that is obtained or obtainable by microionization. In some embodiments, the particle size of the size reduced (e.g., micronized) compound or salt or solvate thereof, is defined by a D50 value of about 0.5 to about 10 microns as measured by an appropriate method known in the art. Dosage forms adapted for administration by inhalation also include particle dusts or mists. Suitable dosage forms wherein the carrier or excipient is a liquid for administration as a nasal spray or drops include aqueous or oil solutions/suspensions of an active (primary and/or secondary) ingredient, which may be generated by various types of metered dose pressurized aerosols, nebulizers, or insufflators. The nasal/inhalation formulations can be administered to a subject in need thereof.

In some embodiments, the dosage forms are aerosol formulations suitable for administration by inhalation. In some of these embodiments, the aerosol formulation contains a solution or fine suspension of a primary active ingredient, secondary active ingredient, and/or pharmaceutically acceptable salt thereof where appropriate and a pharmaceutically acceptable aqueous or non-aqueous solvent. Aerosol formulations can be presented in single or multi-dose quantities in sterile form in a sealed container. For some of these embodiments, the sealed container is a single dose or multi-dose nasal or an aerosol dispenser fitted with a metering valve (e.g., metered dose inhaler), which is intended for disposal once the contents of the container have been exhausted.

Where the aerosol dosage form is contained in an aerosol dispenser, the dispenser contains a suitable propellant under pressure, such as compressed air, carbon dioxide, or an organic propellant, including but not limited to a hydrofluorocarbon. The aerosol formulation dosage forms in other embodiments are contained in a pump-atomizer. The pressurized aerosol formulation can also contain a solution or a suspension of a primary active ingredient, optional secondary active ingredient, and/or pharmaceutically acceptable salt thereof. In further embodiments, the aerosol formulation also contains co-solvents and/or modifiers incorporated to improve, for example, the stability and/or taste and/or fine particle mass characteristics (amount and/or profile) of the formulation. Administration of the aerosol formulation can be once daily or several times daily, for example 2, 3, 4, or 8 times daily, in which 1, 2, 3 or more doses are delivered each time. The aerosol formulations can be administered to a subject in need thereof.

For some dosage forms suitable and/or adapted for inhaled administration, the pharmaceutical formulation is a dry powder inhalable-formulation. In addition to a primary active agent, optional secondary active ingredient, and/or pharmaceutically acceptable salt thereof where appropriate, such a dosage form can contain a powder base such as lactose, glucose, trehalose, mannitol, and/or starch. In some of these embodiments, a primary active agent, secondary active ingredient, and/or pharmaceutically acceptable salt thereof where appropriate is in a particle-size reduced form. In further embodiments, a performance modifier, such as L-leucine or another amino acid, cellobiose octaacetate, and/or metals salts of stearic acid, such as magnesium or calcium stearate. In some embodiments, the aerosol formulations are arranged so that each metered dose of aerosol contains a predetermined amount of an active ingredient, such as the one or more of the compositions, compounds, vector(s), molecules, cells, and combinations thereof described herein.

Dosage forms adapted for vaginal administration can be presented as pessaries, tampons, creams, gels, pastes, foams, or spray formulations. Dosage forms adapted for rectal administration include suppositories or enemas. The vaginal formulations can be administered to a subject in need thereof.

Dosage forms adapted for parenteral administration and/or adapted for injection can include aqueous and/or non-aqueous sterile injection solutions, which can contain antioxidants, buffers, bacteriostats, solutes that render the composition isotonic with the blood of the subject, and aqueous and non-aqueous sterile suspensions, which can include suspending agents and thickening agents. The dosage forms adapted for parenteral administration can be presented in a single-unit dose or multi-unit dose containers, including but not limited to sealed ampoules or vials. The doses can be lyophilized and re-suspended in a sterile carrier to reconstitute the dose prior to administration. Extemporaneous injection solutions and suspensions can be prepared in some embodiments, from sterile powders, granules, and tablets. The parenteral formulations can be administered to a subject in need thereof.

For some embodiments, the dosage form contains a predetermined amount of a primary active agent, secondary active ingredient, and/or pharmaceutically acceptable salt thereof where appropriate per unit dose. In an embodiment, the predetermined amount of primary active agent, secondary active ingredient, and/or pharmaceutically acceptable salt thereof where appropriate can be an effective amount, a least effect amount, and/or a therapeutically effective amount. In other embodiments, the predetermined amount of a primary active agent, secondary active agent, and/or pharmaceutically acceptable salt thereof where appropriate, can be an appropriate fraction of the effective amount of the active ingredient.

### Co-Therapies and Combination Therapies

In some embodiments, the pharmaceutical formulation(s) described herein are part of a combination treatment or combination therapy. The combination treatment can include the pharmaceutical formulation described herein and an additional treatment modality. The additional treatment modality can be a chemotherapeutic, a biological therapeutic, surgery, radiation, diet modulation, environmental modulation or therapy, a physical activity modulation, acupuncture, electrical stimulation, physical therapy, and any combination thereof.

In some embodiments, the co-therapy or combination therapy include, but is not limited to, polynucleotides, amino acids, peptides, polypeptides, antibodies, aptamers, ribozymes, hormones, immunomodulators, antipyretics, anxiolytics, antipsychotics, analgesics, antispasmodics, anti-inflammatoires, anti-histamines, anti-infectives, chemotherapeutics, and combinations thereof.

In some embodiments, the co-therapy or combination therapy is or includes an anti-infective. Exemplary anti-infectives are described in greater detail elsewhere herein.

In certain embodiments, the co-therapy or combination therapy is or includes a peptidoglycan-specific hydrolase.

In certain embodiments, the co-therapy or combination therapy is or includes an analgesic. In certain embodiments, the co-therapy or combination therapy is or includes an anti-pyretic. In some embodiments, the co-therapy or combination therapy is or includes an anti-infective and a peptidoglycan-specific hydrolase. In some embodiments, the co-therapy or combination therapy is or includes a peptidoglycan-specific hydrolase, an anti-infective, and an analgesic. In some embodiments, the co-therapy or combination therapy is or includes a peptidoglycan-specific hydrolase, an anti-infective, an analgesic, and an anti-pyretic. In some embodiments, the co-therapy or combination therapy includes an antibody or fragment thereof of the present disclosure and an anti-infective

In some embodiments, the anti-infective agent comprises doxycycline, amoxicillin, cefuroxime, cefotaxime, azlocillin, penicillin, erythromycin, ceftriaxone, or any combination thereof.

In some embodiments, the peptidoglycan-specific hydrolase is a bacterial peptidoglycan specific hydrolase. Exemplary peptidoglycan hydrolases include N-Acetylmuramyl-L-alanine amidases hydrolyses, N-acetylglucosaminidases, and N-acetyl-β-D-muramidases. See also, e.g., Vollmer et al., FEMS Microbiol. Rev. 2008, 32(2), 259-286; and Chodisetti et al., Proc. Nat. Acad. Sci. U.S.A. 2019, 116(16), 7825-7830. In some embodiments, the peptidoglycan-specific hydrolase includes an amidase or a lysozyme.

Suitable antipyretics include, but are not limited to, non-steroidal anti-inflammatoires (e.g., ibuprofen, naproxen, ketoprofen, and nimesulide), aspirin and related salicylates (e.g., choline salicylate, magnesium salicylate, and sodium salicylate), paracetamol/acetaminophen, metamizole, nabumetone, phenazone, and quinine.

Suitable analgesics include, but are not limited to, paracetamol/acetaminophen, nonsteroidal anti-inflammantories (e.g., ibuprofen, naproxen, ketoprofen, and nimesulide), COX-2 inhibitors (e.g., rofecoxib, celecoxib, and etoricoxib), opioids (e.g. morphine, codeine, oxycodone, hydrocodone, dihydromorphine, pethidine, buprenorphine), tramadol, norepinephrine, flupirtine, nefopam, orphenadrine, pregabalin, gabapentin, cyclobenzaprine, scopolamine, methadone, ketobemidone, piritramide, and aspirin and related salicylates (e.g., choline salicylate, magnesium salicylate, and sodium salicylate).

### Administration of the Pharmaceutical Formulations

The pharmaceutical formulations or dosage forms thereof described herein can be administered one or more times hourly, daily, monthly, or yearly (e.g., 1, 2, 3, 4, 5, 6, 7, 8, 9, 10, 11, 12, 13, 14, 15, 16, 17, 18, 19, 20, or more times hourly, daily, monthly, or yearly). In some embodiments, the pharmaceutical formulations or dosage forms thereof described herein can be administered continuously over a period of time ranging from minutes to hours to days. Devices and dosages forms are known in the art and described herein that are effective to provide continuous administration of the pharmaceutical formulations described herein. In some embodiments, the first one or a few initial amount(s) administered can be a higher dose than subsequent doses. This is typically referred to in the art as a loading dose or doses and a maintenance dose, respectively. In some embodiments, the pharmaceutical formulations can be administered such that the doses over time are tapered (increased or decreased) overtime so as to wean a subject gradually off of a pharmaceutical formulation or gradually introduce a subject to the pharmaceutical formulation.

As previously discussed, the pharmaceutical formulation can contain a predetermined amount of a primary active agent, secondary active agent, and/or pharmaceutically acceptable salt thereof where appropriate. In some of these embodiments, the predetermined amount can be an appropriate fraction of the effective amount of the active ingredient. Such unit doses may therefore be administered once or more than once a day, month, or year (e.g., 1, 2, 3, 4, 5, 6, or more times per day, month, or year). Such pharmaceutical formulations may be prepared by any of the methods well known in the art.

Where co-therapies or multiple pharmaceutical formulations are to be delivered to a subject, the different therapies or formulations can be administered sequentially or simultaneously. Sequential administration is administration where an appreciable amount of time occurs between administrations, such as more than about 15, 20, 30, 45, 60 minutes or more. The time between administrations in sequential administration can be on the order of hours, days, months, or even years, depending on the active agent present in each administration. Simultaneous administration refers to administration of two or more formulations at the same time or substantially at the same time (e.g., within seconds or just a few minutes apart), where the intent is that the formulations be administered together at the same time.

### Kits

Described in example embodiments herein are kits, such as combination kits, that contain one or more compounds, compositions, formulations, devices, for performing a method described herein. As used herein, the terms “combination kit” or “kit of parts” refers to the compounds, compositions, formulations, particles, devices, and any additional components that are used to collect samples, store samples, ship samples, test samples, package, sell, market, deliver, and/or administer an element, combination of elements or a single element, such as the active ingredient, contained therein. Such additional components include, but are not limited to, packaging, syringes, blister packages, bottles, solutions, detection reagents, swabs, collection vials, collection tubes, labels and the like.

In some embodiments, the kit comprises (a) the antibody or fragment thereof capable of specifically binding a Borreliae peptidoglycan; (b) a polynucleotide encoding the antibody or fragment thereof; (c) a vector comprising the polynucleotide; (d) a cell or population comprising the antibody or fragment thereof, polynucleotide, vector, or any combination thereof; (e) a pharmaceutical formulation comprising the antibody or fragment thereof, polynucleotide, vector, cell or population thereof, or any combination thereof, and a pharmaceutically acceptable carrier; or (f) any combination of (a)-(e). In some embodiments, the Borreliae peptidoglycan to which the antibody or fragment thereof is capable of specifically binding is a Borrelia burgdorferi, Borrelia afzelii, Borrelia garinii, Borrelia japonica, Borrelia andersonii, Borrelia bissettii, Borrelia sinica, Borrelia turdi, Borrelia tanukii, Borrelia valaisiana, Borrelia lusitaniae, Borrelia spielmanii, Borrelia miyamotoi, and/or a Borrelia Lonestari peptidoglycan.

In some embodiments, the combination kit also includes instructions printed on or otherwise contained in a tangible medium of expression. The instructions can provide information regarding the content of the compounds, compositions, formulations, particles, and/or devices contained in the kit, safety information regarding the content of the compounds, compositions, formulations, particles, and/or devices contained in the kit information regarding the dosages, indications for use, and/or recommended treatment regimen(s) for the compounds, compositions, formulations, particles, and/or devices contained in the kit. In some embodiments, the instructions can provide directions for administering or otherwise using the compounds, compositions, formulations, particles, and devices described herein or a combination thereof to a subject in need thereof. In some embodiments, the subject in need thereof is in need of diagnosing, prognosing, monitoring, staging, and/or treating Lyme disease and/or a Borrelial disease and/or Borreliae infection (referred to herein as a “Borreliae infection”). In some embodiments, the Borrelial disease and/or Borreliae infection is a Borrelia burgdorferi, Borrelia afzelii, Borrelia garinii, Borrelia japonica, Borrelia andersonii, Borrelia bissettii, Borrelia sinica, Borrelia turdi, Borrelia tanukii, Borrelia valaisiana, Borrelia lusitaniae, Borrelia spielmanii, Borrelia miyamotoi, and/or a Borrelia Lonestar disease and/or infection.

### Methods of Use

Described in certain example embodiments herein are methods of detecting and/or quantifying Borreliae peptidoglycan, and/or diagnosing a Borreliae infection, Borrelial disease, and/or Lyme disease in a subject in need, using the antibodies or fragments thereof described herein. In some embodiments, the method of detecting, quantifying, and/or diagnosing comprises contacting the antibody or fragment thereof described herein with a biological sample from a subject in need and detecting and/or quantifying the amount of Borreliae peptidoglycan in the sample. In some embodiments, the method of detecting and/or diagnosing further comprises treating a subject with one or more therapeutic agents.

Described in certain example embodiments herein are methods of treating a Borreliae peptidoglycan disease or symptom thereof, a Borreliae infection or symptom thereof, a Borrelial disease, and/or Lyme disease or symptom thereof in a subject in need, as well as methods of reducing, inhibiting, or ameliorating one or more symptoms of Lyme and/or Borrelial disease in a subject in need, using the antibodies or fragments thereof described herein. In some embodiments, the Borrelial disease and/or Borreliae infection is a Borrelia burgdorferi, Borrelia afzelii, Borrelia garinii, Borrelia japonica, Borrelia andersonii, Borrelia bissettii, Borrelia sinica, Borrelia turdi, Borrelia tanukii, Borrelia valaisiana, Borrelia lusitaniae, Borrelia spielmanii, Borrelia miyamotoi, and/or a Borrelia Lonestar disease and/or infection.

### Diagnostic Applications

Disclosed herein are methods of diagnosing a Borrelialdisease, e.g., Lyme disease (including Lyme disease-related pathologies, e.g., arthritis, neurological disorder, and/or inflammatory condition) in a subject in need thereof comprising contacting an anti-peptidoglycan agent, e.g., an antibody or fragment thereof of the present disclosure that specifically binds to a Borreliae peptidoglycan with a biological sample of the subject and measuring the level of Borreliae peptidoglycan in the sample. In some embodiments, the Borreliae peptidoglycan is a Borrelia burgdorferi, Borrelia afzelii, Borrelia garinii, Borrelia japonica, Borrelia andersonii, Borrelia bissettii, Borrelia sinica, Borrelia turdi, Borrelia tanukii, Borrelia valaisiana, Borrelia lusitaniae, Borrelia spielmanii, Borrelia miyamotoi, and/or a Borrelia Lonestar peptidoglycan. In some embodiments, a method of diagnosing Lyme disease in a subject in need thereof comprises contacting an antibody against an anti-peptidoglycan antibody (i.e., anti-idiotypic antibody) with a biological sample of the subject and measuring the level of anti-peptidoglycan antibody in the sample.

Also disclosed herein are compositions, such as any of those described in greater detail elsewhere herein, for diagnosing a Borrelial disease or Borreliae infection, such as Lyme disease (including Lyme disease-related pathologies, e.g., arthritis, neurological disorder, and/or inflammatory condition) includes an agent for measuring the level of Borreliae peptidoglycan or the level of antibodies directed against Borreliae peptidoglycan. In some embodiments, the Borreliae peptidoglycan is a Borrelia burgdorferi, Borrelia afzelii, Borrelia garinii, Borrelia japonica, Borrelia andersonii, Borrelia bissettii, Borrelia sinica, Borrelia turdi, Borrelia tanukii, Borrelia valaisiana, Borrelia lusitaniae, Borrelia spielmanii, Borrelia miyamotoi, and/or a Borrelia Lonestar peptidoglycan. Non-limiting examples of such agents include antibodies, or antigen-binding fragments thereof, that specifically bind to Borreliae peptidoglycan or antibodies directed against the Borreliae peptidoglycan. In some embodiments, any assay that can measure antigen-antibody binding could be used to measure the level of Borreliae peptidoglycan or the level of antibodies directed against the Borreliae peptidoglycan. Non-limiting examples of such assays include Surface Plasmon Resonance, Standard Dot and line Immunoblot techniques, immuno-PCR, and PCR-ELISA. Additional assays that can be used with the present disclosure include colorimetric assay, gas liquid chromatography, and mass spectrometry techniques.

In some embodiments, the level of Borreliae peptidoglycan in a biological sample is increased by at least about 5%, at least about 10%, at least about 20%, at least about 30%, at least about 40%, at least about 50%, at least about 60%, at least about 70%, at least about 80%, at least about 90%, at least about 100%, at least about 150%, at least about 200%, at least about 300% or more compared to a corresponding level in a reference sample or control (e.g., sample of a subject who does not have Lyme disease). The subject who exhibits an increased level or amount of Borreliae peptidoglycan can be identified as a subject suspected of having a Borreliae infection, Borrelial disease, and/or Lyme disease. In some embodiments, the subject who exhibits an increased level or amount of Borrelia burgdorferi, Borrelia afzelii, Borrelia garinii, Borrelia japonica, Borrelia andersonii, Borrelia bissettii, Borrelia sinica, Borrelia turdi, Borrelia tanukii, Borrelia valaisiana, Borrelia lusitaniae, Borrelia spielmanii, Borrelia miyamotoi, and/or a Borrelia Lonestar peptidoglycan can be identified as a subject suspected of having a Borreliae infection, Borrelial disease, and/or Lyme disease. In some embodiments, the subject who exhibits an increased level of Borreliae peptidoglycan can be identified as a subject suspected of having Lyme disease. In some embodiments, the subject who exhibits an increased level or amount of Borrelia burgdorferi, Borrelia afzelii, and/or Borrelia garinii peptidoglycan can be identified as a subject suspected of having a Borrelial disease, a Borrelia infection, and/or Lyme disease.

In some embodiments, the level or amount of antibody directed against Borreliae species peptidoglycan (also referred to herein as an “anti-peptidoglycan antibody”) in a biological sample is increased by at least about 5%, at least about 10%, at least about 20%, at least about 30%, at least about 40%, at least about 50%, at least about 60%, at least about 70%, at least about 80%, at least about 90%, at least about 100%, at least about 150%, at least about 200%, at least about 300% or more compared to a corresponding level in a reference sample (e.g., sample of a subject who does not have Lyme disease or a Borrelial disease or infection). In some embodiments, a subject with an increased level or amount of antibody directed against Borreliae peptidoglycan has increased level or amount of Borreliae peptidoglycan. In some embodiments, the Borreliae peptidoglycan is a Borrelia burgdorferi, Borrelia afzelii, Borrelia garinii, Borrelia japonica, Borrelia andersonii, Borrelia bissettii, Borrelia sinica, Borrelia turdi, Borrelia tanukii, Borrelia valaisiana, Borrelia lusitaniae, Borrelia spielmanii, Borrelia miyamotoi, and/or a Borrelia Lonestar peptidoglycan. Accordingly, in certain embodiments, a subject who exhibits an increased level or amount of antibody directed against Borreliae peptidoglycan can be identified as a subject suspected of having a Borrelial disease, Borreliae infection, and/or Lyme disease.

In some embodiments, the reference sample (e.g., reference biological sample) can be obtained from the same subject wherein the reference sample was obtained prior to the onset of a Borrelial disease, Borreliae infection, and/or Lyme disease (i.e., prior to Borreliae infection (e.g., a Borrelia burgdorferi, Borrelia afzelii, Borrelia garinii, Borrelia japonica, Borrelia andersonii, Borrelia bissettii, Borrelia sinica, Borrelia turdi, Borrelia tanukii, Borrelia valaisiana, Borrelia lusitaniae, Borrelia spielmanii, Borrelia miyamotoi, and/or a Borrelia Lonestar infection). In other embodiments, the reference sample can be obtained from a control subject or a population of control subjects who do not have a Borrelial disease, Borreliae infection, and/or Lyme disease, e.g., health control subjects.

In some embodiments, a biological sample comprises a tissue, cell, or bodily fluid (e.g., blood, plasma, saliva, synovial fluid, cerebral spinal fluid, or urine). In certain embodiments, a biological sample comprises serum, plasma, synovial fluid, cerebral spinal fluid, urine, or any combination thereof.

In certain embodiments, the level or amount of Borreliae peptidoglycan (e.g., a Borrelia burgdorferi, Borrelia afzelii, Borrelia garinii, Borrelia japonica, Borrelia andersonii, Borrelia bissettii, Borrelia sinica, Borrelia turdi, Borrelia tanukii, Borrelia valaisiana, Borrelia lusitaniae, Borrelia spielmanii, Borrelia miyamotoi, and/or a Borrelia Lonestar peptidoglycan) in a synovial fluid of a subject suspected of having a Borrelial disease, Borreliae infection, and/or Lyme disease is increased by at least about 5%, at least about 10%, at least about 20%, at least about 30%, at least about 40%, at least about 50%, at least about 60%, at least about 70%, at least about 80%, at least about 90%, at least about 100%, at least about 150%, at least about 200%, at least about 300% or more compared to a corresponding level in a reference sample (e.g., sample of a subject who does not have a Borrelial disease, Borreliae a infection, and/or Lyme disease).

In some embodiments, an increase in the level or amount of the anti-peptidoglycan antibody or fragment thereof of the present disclosure is observed both in the synovial fluid and/or serum. Accordingly, in certain embodiments, the level of anti-peptidoglycan antibody protein in a synovial fluid and/or serum of a subject suspected having a Borrelial disease, Borreliae infection, and/or Lyme disease is increased by at least about 5%, at least about 10%, at least about 20%, at least about 30%, at least about 40%, at least about 50%, at least about 60%, at least about 70%, at least about 80%, at least about 90%, at least about 100%, at least about 150%, at least about 200%, at least about 300% or more compared to a corresponding level or amount in a reference sample (e.g., sample of a subject who does not have a Borrelialdisease, Borreliae infection, and/or Lyme disease).

In some embodiments, an increase in the level or amount of the anti-peptidoglycan antibody or fragment thereof of the present disclosure is observed both in the synovial fluid, serum, and urine. Accordingly, in certain embodiments, the level of anti-peptidoglycan antibody protein in a synovial fluid, serum and/or urine of a subject suspected having a Borrelial disease, Borreliae infection, and/or Lyme disease is increased by at least about 5%, at least about 10%, at least about 20%, at least about 30%, at least about 40%, at least about 50%, at least about 60%, at least about 70%, at least about 80%, at least about 90%, at least about 100%, at least about 150%, at least about 200%, at least about 300% or more compared to a corresponding level or amount in a reference sample (e.g., sample of a subject who does not have a Borrelial disease, Borreliae infection, and/or Lyme disease).

In some embodiments, the level or amount of anti-peptidoglycan antibody in the serum is lower than the level or amount of anti-peptidoglycan antibody observed in the synovial fluid. In certain embodiments, the level of anti-peptidoglycan antibody in the serum is lower than the corresponding level in the synovial fluid by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or more.

In some embodiments, the level or amount of anti-peptidoglycan antibody in the serum is higher than the level or amount of anti-peptidoglycan antibody observed in the synovial fluid. In certain embodiments, the level or amount of anti-peptidoglycan antibody in the serum is higher than the corresponding level or amount in the synovial fluid by at least about 5%, at least about 10%, at least about 20%, at least about 30%, at least about 40%, at least about 50%, at least about 60%, at least about 70%, at least about 80%, at least about 90%, at least about 100%, at least about 150%, at least about 200%, at least about 300% or more.

In some embodiments, the level or amount of Borreliae peptidoglycan (e.g., Borreliae peptidoglycan (e.g., a Borrelia burgdorferi, Borrelia afzelii, Borrelia garinii, Borrelia japonica, Borrelia andersonii, Borrelia bissettii, Borrelia sinica, Borrelia turdi, Borrelia tanukii, Borrelia valaisiana, Borrelia lusitaniae, Borrelia spielmanii, Borrelia miyamotoi, and/or a Borrelia Lonestar peptidoglycan) in the synovial fluid of a subject suspected of having a Borrelial disease, Borreliae infection, and/or Lyme disease is at least about 0.5 μg/mL, at least about 1 μg/mL, at least about 5 μg/mL, at least about 10 μg/mL, at least about 20 μg/mL, at least about 30 μg/mL, at least about 40 μg/mL, at least about 50 μg/mL, at least about 60 μg/mL, at least about 70 μg/mL, at least about 80 μg/mL, at least about 90 μg/mL, at least about 100 μg/mL, at least about 150 μg/mL, at least about 200 μg/mL or more.

In some embodiments, methods disclosed herein can be used to diagnose the different stages of Lyme disease infection (i.e., Stages 1, 2, and 3, see supra). As shown in the Examples, Applicant has discovered that B. burgdorferi sheds large amounts of antigenic and immunogenic peptidoglycan fragments during its growth (almost 50% per generation). Accordingly, in some embodiments, by measuring the level of B. burgdorferi peptidoglycan (either directly or indirectly through the level of anti-peptidoglycan), diagnostic methods disclosed herein can be used to diagnose both early and late stage Lyme disease infection. The ability to diagnose early stage infection, for example within about 1 week, within about 2 weeks, within about 3 weeks is particularly advantageous as early treatment can mitigate and/or prevent the more serious symptoms of Lyme disease (e.g., arthritis or neuroborreliosis). Currently, there are no reliable methods for detecting early stage Lyme disease infection.

In some embodiments, in addition to increased levels of Borreliae peptidoglycan and/or antibodies directed against Borreliae peptidoglycan, a subject suspected of having a Borrelial disease, Borreliae infection, and/or Lyme disease has increased level of one or more pro-inflammatory mediators in a biological sample (e.g., synovial fluid, serum, or urine). In certain embodiments, the level of pro-inflammatory mediators is increased by at least about 5%, at least about 10%, at least about 20%, at least about 30%, at least about 40%, at least about 50%, at least about 60%, at least about 70%, at least about 80%, at least about 90%, at least about 100%, at least about 150%, at least about 200%, at least about 300% or more compared to a corresponding level in a reference sample (e.g., sample of a subject who does not have a Borrelial disease, Borreliae infection, and/or Lyme disease).

Non-limiting examples of inflammatory mediators include TNF-α, IL-1α, IL-1β, IL-6, IL-8, IFN-γ, IL-17A, IL-17F, or combinations thereof. In some embodiments, the level of pro-inflammatory mediators in the synovial fluid is greater than the corresponding level in the serum in a subject suspected of having a Borrelial disease, Borreliae infection, and/or Lyme disease.

In some embodiments, the level or amount of Borreliae peptidoglycan, the level of anti-peptidoglycan antibody, and/or the level of proinflammatory mediators is measured by an immunohistochemistry, a Western blotting, a radioimmunoassay, an enzyme linked immunosorbent assay (ELISA), a radioimmunodiffusion, an immunoprecipitation assay, an Ouchterlony immunodiffusion method, a rocket immunoelectrophoresis, a tissue immunostaining method, a complement fixation assay, FACS, a protein chip, Surface Plasmon Resonance, Standard Dot and line Immunoblot techniques, PCR-ELISA, immuno-PCR, colorimetric assay, gas liquid chromatography, and mass spectrometry techniques.

In some embodiments, the level of Borreliae peptidoglycan is measured by an assay using an anti-peptidoglycan antibody or innate immunity protein disclosed herein. In some embodiments, the Borreliae peptidoglycan is a Borrelia burgdorferi, Borrelia afzelii, Borrelia garinii, Borrelia japonica, Borrelia andersonii, Borrelia bissettii, Borrelia sinica, Borrelia turdi, Borrelia tanukii, Borrelia valaisiana, Borrelia lusitaniae, Borrelia spielmanii, Borrelia miyamotoi, and/or a Borrelia Lonestar peptidoglycan.

In some embodiments, the diagnosis of the present disclosure can be combined with one or more known diagnostic methods for a Borrelialdisease, Borreliae infection, and/or Lyme disease, such as total IgG in whole cell sonicate as measured by immunoblot and/or ELISA, or VIsE C6 ELISA.

In some embodiments, one or more diagnostic tests can be used to rule out other serious medical conditions that can cause similar symptoms of a Borrelial disease, Borreliae infection, and/or Lyme disease, e.g., other forms of arthritis, such as rheumatoid arthritis, osteoarthritis, ankylosing spondylitis, or gout. In a situation where the subject has other serious medical conditions, the subject can be treated for the other serious medical conditions only without the treatment for Lyme disease. In other embodiments, the subject can be treated for the other serious medical conditions along with the Borrelial disease, Borreliae infection, and/or Lyme disease.

In some embodiments, methods disclosed herein can be used to distinguish active Borreliae (e.g., a Borrelia burgdorferi, Borrelia afzelii, Borrelia garinii, Borrelia japonica, Borrelia andersonii, Borrelia bissettii, Borrelia sinica, Borrelia turdi, Borrelia tanukii, Borrelia valaisiana, Borrelia lusitaniae, Borrelia spielmanii, Borrelia miyamotoi, and/or a Borrelia Lonestar disease or infection) infection from previous exposure. Borrelia peptidoglycan fragments are released only during active growth of the bacteria, and detection of these unique epitopes can delineate current from previous exposure.

In some embodiments, the method further includes diagnosing, monitoring, staging, and/or prognosing a Borreliae infection, Borrelial disease and/or Lyme disease or a symptom thereof in a subject from which the sample was obtained. In some embodiments, the method includes obtaining two or more samples from the same subject, where one or more samples is/are obtained at two or more different time points. In some embodiments, any two points are 1-100 or more minutes, days, weeks, months, or years apart from each other. In some embodiments, the amount and/or types of peptidoglycans or fragment thereof detected in the sample is to determine a diagnosis, prognosis, and/or used to monitor Borreliae infection, Borrelial disease, and/or Lyme disease or Borreliae infection, Borrelial disease, and/or Lyme disease progression and/or response to a treatment or therapy.

The terms “diagnosis,” “prognosis”, and “monitoring” are commonplace and well-understood in medical practice. By means of further explanation and without limitation the term “diagnosis” generally refers to the process or act of recognising, deciding on or concluding on a disease or condition in a subject on the basis of symptoms and signs and/or from results of various diagnostic procedures (such as, for example, from knowing the presence, absence and/or quantity of one or more biomarkers characteristic of the diagnosed disease or condition). The terms “prognosing” or “prognosis” generally refer to an anticipation on the progression of a disease or condition and the prospect (e.g., the probability, duration, and/or extent) of recovery. A good prognosis of the diseases or conditions taught herein may generally encompass anticipation of a satisfactory partial or complete recovery from the diseases or conditions, preferably within an acceptable time period. A good prognosis of such may more commonly encompass anticipation of not further worsening or aggravating of such, preferably within a given time period. A poor prognosis of the diseases or conditions as taught herein may generally encompass anticipation of a substandard recovery and/or unsatisfactorily slow recovery, or to substantially no recovery or even further worsening of such.

In some embodiments, the method further includes staging a Borrelial disease and/or Lyme disease, infection with a Borrelia burgdorferi organism, or both, or a symptom thereof. The term “staging” is used in accordance with its commonplace use in the medical and/or veterinary fields, and refers to identifying what stage or phase of a disease progression a subject is in. In some embodiments, the level, amount, and/or type of Borreliae peptidoglycan indicates alone or when considered with one or more other disease indicators the stage of Borrelial disease, Lyme disease or B. burgdorferi infection as subject is in. In some embodiments, the subject is asymptomatic.

In some embodiments, the method is specific to detecting B. Burgdorferi. In some embodiments, the method does not detect other Borrelia species, other spirochetes, and/or other bacteria, and/or other microorganisms. In some embodiments, the method is specific to detecting Borreliae species against other spirochetes, bacteria, and/or other microorganisms. In some embodiments, the method is specific to detecting Borrelia against other spirochetes, bacteria, and/or other microorganisms.

In some embodiments, a method of diagnosing Borreliae infection (e.g., a Borrelia burgdorferi, Borrelia afzelii, Borrelia garinii, Borrelia japonica, Borrelia andersonii, Borrelia bissettii, Borrelia sinica, Borrelia turdi, Borrelia tanukii, Borrelia valaisiana, Borrelia lusitaniae, Borrelia spielmanii, Borrelia miyamotoi, and/or a Borrelia Lonestar infection) in a subject is provided, the method including; a) contacting a sample obtained from the individual with an agent specific for one or more Borreliae species peptidoglycan (e.g., an antibody or fragment thereof of the present disclosure), which agents are operably linked to a detectable moiety in a reaction mixture; b) washing the reaction mixture free of unbound agent; c) analyzing the reaction mixture for the presence of bound peptidoglycan-specific agent to determine the presence of Borreliae peptidoglycan, wherein the presence is indicative of a Borreliae infection; and d) diagnosing the individual with Borreliae infection if the presence of one peptidoglycan at a level greater than that of a reference sample is detected. In some embodiments, the method further includes collecting at least one biological sample from the subject prior to (a). In some embodiments, infection can be detected within about 1 week, within about 2 weeks, within about 3 weeks after the initial infection.

### Therapeutic Applications

Disclosed herein are also methods of treating, controlling, ameliorating, or reducing a Borrelial disease, Borreliae infection, and/or Lyme disease and/or a symptom thereof (e.g., one or more symptoms associated with Lyme disease, e.g., arthritis) in a subject in need thereof (e.g., a subject identified as having Lyme disease using the diagnostic methods disclosed herein). In some embodiments, the treatment of Borrelial disease and Lyme disease comprises administering to the subject an anti-body that binds Borrelia peptidoglycan (i.e., an anti-Borreliae peptidoglycan antibody or fragment thereof (e.g., an antigen binding fragment thereof) described in greater detail elsewhere herein), a polynucleotide encoding the an anti-Borreliae peptidoglycan antibody or fragment thereof, and/or a cell capable of producing the an anti-Borreliae peptidoglycan antibody or fragment thereof. In some embodiments the Borrelial disease, Borreliae infection, and/or Lyme disease is a Borrelia burgdorferi, Borrelia afzelii, Borrelia garinii, Borrelia japonica, Borrelia andersonii, Borrelia bissettii, Borrelia sinica, Borrelia turdi, Borrelia tanukii, Borrelia valaisiana, Borrelia lusitaniae, Borrelia spielmanii, Borrelia miyamotoi, and/or a Borrelia Lonestar disease or infection. In some embodiments, the Borreliae peptidoglycan is Lyme disease is a Borrelia burgdorferi, Borrelia afzelii, Borrelia garinii, Borrelia japonica, Borrelia andersonii, Borrelia bissettii, Borrelia sinica, Borrelia turdi, Borrelia tanukii, Borrelia valaisiana, Borrelia lusitaniae, Borrelia spielmanii, Borrelia miyamotoi, and/or a Borrelia Lonestar peptidoglycan.

In some embodiments, the antibody or fragment thereof (e.g., antigen-binding fragment thereof, specifically binds to the Borreliae peptidoglycan. In some embodiments, an anti-peptidoglycan antibody or fragment thereof binds to Borreliae peptidoglycan and reduces the activity of Borreliae peptidoglycan.

In some embodiments, the method includes treating a subject for infection or a symptom thereof or a secondary condition thereof with one or more other species of Borrelia including, but not limited to, Borrelia japonica, Borrelia andersonii, Borrelia bissettii, Borrelia sinica, Borrelia turdi, Borrelia tanukii, Borrelia valaisiana, Borrelia lusitaniae, Borrelia spielmanii, Borrelia miyamotoi, Borrelia garinii, Borrelia afzelii, or Borrelia lonestar. In some embodiments, this is in addition to treating the subject for B. burgdorferi for infection or a symptom thereof or a secondary condition thereof.

As shown in the Examples, Applicant has discovered that Borreliae peptidoglycan as exemplified by B. burgdorferi peptidoglycan can induce cells (e.g., human peripheral blood mononuclear cells) to produce pro-inflammatory mediators upon stimulation. Accordingly, in some embodiments, an antagonist (e.g., an anti-peptidoglycan antibody disclosed herein), when administered to a subject, can bind and neutralize the activity of B. burgdorferi peptidoglycan, and thereby, reduce the release of such pro-inflammatory mediators. And, by reducing the release of pro-inflammatory mediators, methods disclosed herein can treat, control, ameliorate, or reduce Lyme disease (e.g., one or more symptoms associated with Lyme disease, e.g., arthritis).

In some embodiments, the level of pro-inflammatory mediators in a subject suspected of having Lyme disease is reduced by at least about 5%, at least about 10%, at least about 20%, at least about 30%, at least about 40%, at least about 50%, at least about 60%, at least about 70%, at least about 80%, at least about 90% or more compared to a reference (e.g., corresponding level in a Lyme disease subject not treated with an antagonist disclosed herein (e.g., the anti-peptidoglycan antibody), e.g., corresponding level in the subject immediately prior to the antagonist administration).

Non-limiting examples of inflammatory mediators include TNF-α, IL-1α, IL-1β, IL-6, IL-8, IFN-γ, CCL2, CCL3, CXCL10, IL-17A, IL-17F, or combinations thereof. In some embodiments, the level of pro-inflammatory mediators in the synovial fluid is greater than the corresponding level in the serum in a subject suspected of having Lyme disease.

In some embodiments, therapeutic methods disclosed herein can comprise administering multiple agents to a subject (e.g., anti-peptidoglycan antibody and a secondary active agent (including but not limited to a peptidoglycan-specific hydrolase, an anti-infective, an analgesic, and/or an anti-pyretic). In such embodiments, the multiple antagonists can be administered concurrently or sequentially. In some embodiments, therapeutic methods disclosed herein can be used in conjunction with one or more additional agents for the treatment of Lyme disease. In certain embodiments, the one or more additional agents comprise an antibiotic. Non-limiting examples of antibiotics include doxycycline, amoxicillin, ceftriaxone, or combinations thereof. Other additional agents, co-therapies, and co-treatments are described in greater detail elsewhere herein.

In some embodiments, anti-peptidoglycan antibody) is administered to the subject intravenously, orally, parenterally, intrathecally, intra-cerebroventricularly, pulmonarily, intramuscularly, subcutaneously, intravitreally, or intraventricularly.

In some embodiments, the is further treated with a secondary active agent, co-therapy, and/or combination therapy, such as any of those described in greater detail elsewhere herein. In some embodiments, the anti-Borreliae peptidoglycan antibody, secondary active agent, co-treatment, and/or co-therapy is administered or otherwise applied or delivered to a site of inflammation in the subject in need thereof.

Further embodiments are illustrated in the following Examples which are given for illustrative purposes only and are not intended to limit the scope of the invention.

### EXAMPLES

Now having described the embodiments of the present disclosure, in general, the following Examples describe some additional embodiments of the present disclosure. While embodiments of the present disclosure are described in connection with the following examples and the corresponding text and figures, there is no intent to limit embodiments of the present disclosure to this description. On the contrary, the intent is to cover all alternatives, modifications, and equivalents included within the spirit and scope of embodiments of the present disclosure. The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

### Example 1

Rabbit Immunization Strategy and Response to Borrelia burgdorferi Muropeptides.

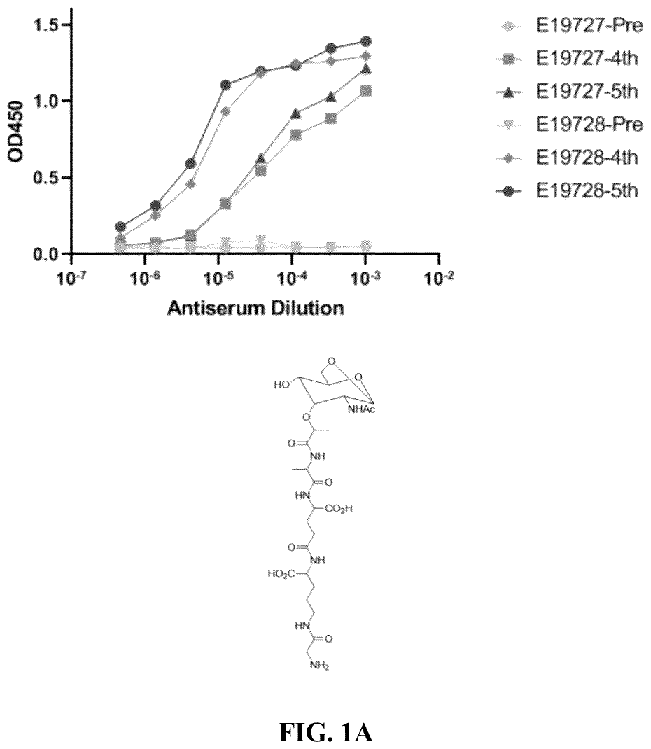

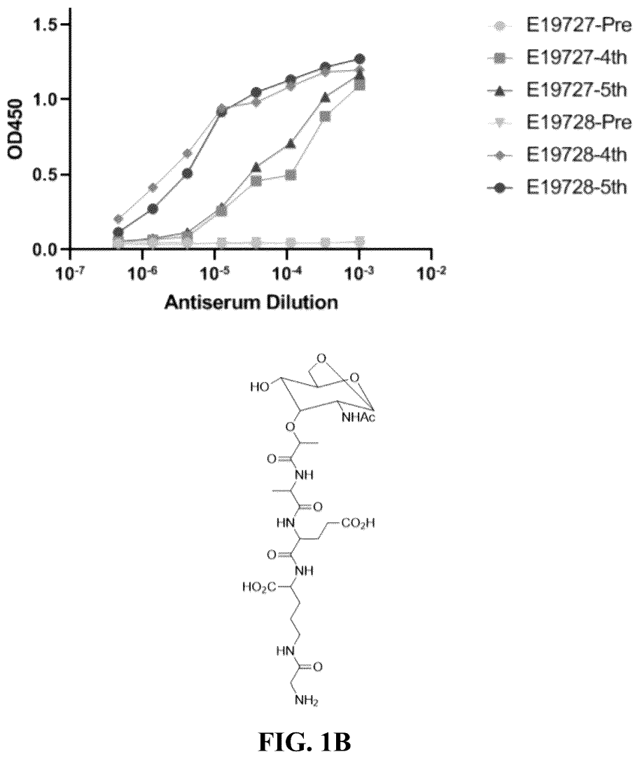

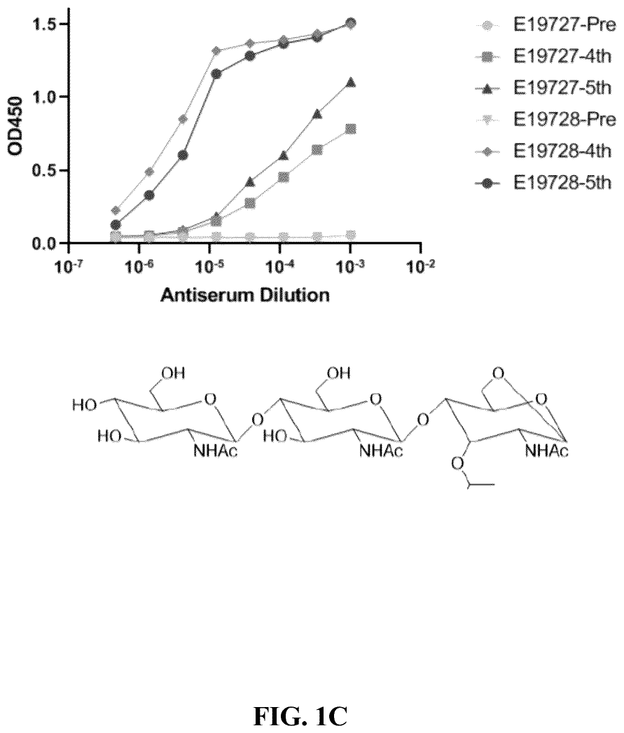

Two New Zeeland White Rabbits were immunized every 6 weeks with 5 doses of B. burgdorferi peptidoglycan that had been digested in the fragments (muropeptides). Both animals were tested for antibody titers against AnhMurNAc-L-Ala-D-Glu-alpha-L-Orn, (FIG. 1A) AnhMurNAc-L-Ala-D-Glu-gamma-L-Orn (FIG. 1B), or GlcNAc-GlcNAc-AnhMurNAc (FIG. 1C). Animal E19728 was used to collect splenocytes and test >500 B cell clones for specificity against each fragment above.

### Refinement of B Cell Clones Specific for Muropeptides of Interest and for Counterselection.

After screening >500 B cell clones, a subset was used for counter selection against the commonly released peptidoglycan fragments AnhMurNAc-L-Ala-D-Glu-L-Lys (FIG. 2, gray) or AnhMurNAc-L-Ala-D-Glu-mDAP (FIG. 2, black). 1A4, 1E10, 2G10, and 2H10 were selected for lab-scale recombinant mAb production based on their specificity for target antigens of interest.

Purification and Analysis of Select mAb on Released Peptidoglycan by Live B. burgdorferi.

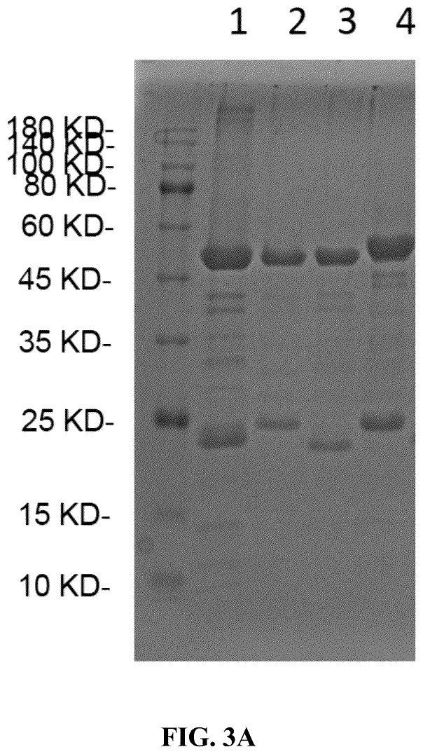

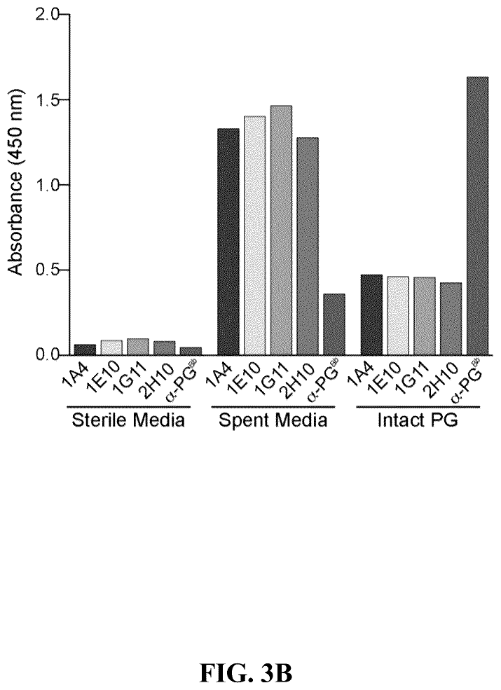

Purified mAb H and L chains were analyzed by SDS-PAGE. Lanes 1~4 correspond to mAbs 1A4, 1E10, 2G10, and 2H10, respectively (FIG. 3A). ELISAs using recombinant mAb to detect released peptidoglycan present in culture supernatants following culture expansion (spent media) or intact peptidoglycan from cells (Intact PG) (FIG. 3B). The results indicate that all four mAb are capable of detecting released peptidoglycan.

### Immuno-PCR to Detect Fragments of Lyme Disease Causing Borrelia Species Peptidoglycan.

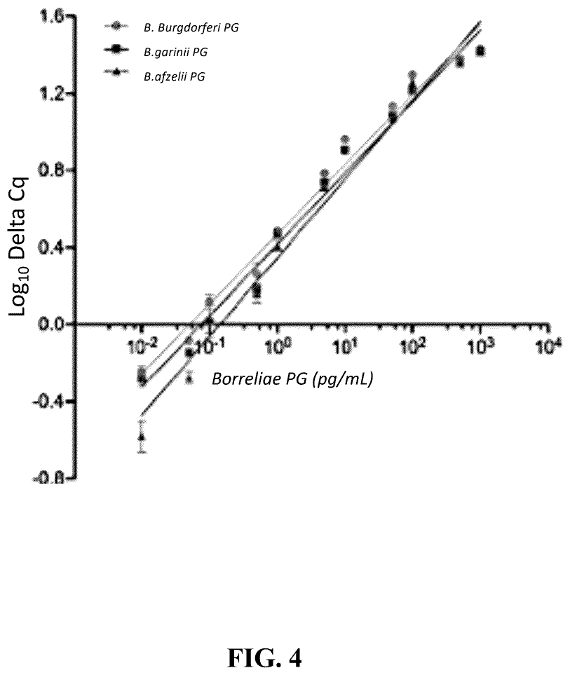

Applicant used a cocktail of 1A4, 1E10, 2G10, and 2H10 as both capture and detection monoclonal antibodies by immune-PCR. This was an effective strategy to detect fragments of peptidoglycan purified from the Lyme disease-causing organisms Borrelia burgdorferi, Borrelia afzelii, and Borrelia garinii (FIG. 4).

### Combinations of Recombinant Monoclonal Antibodies are Capable of Detecting Fragments of Released Peptidoglycan in the Serum of Experimentally Infected Mice.

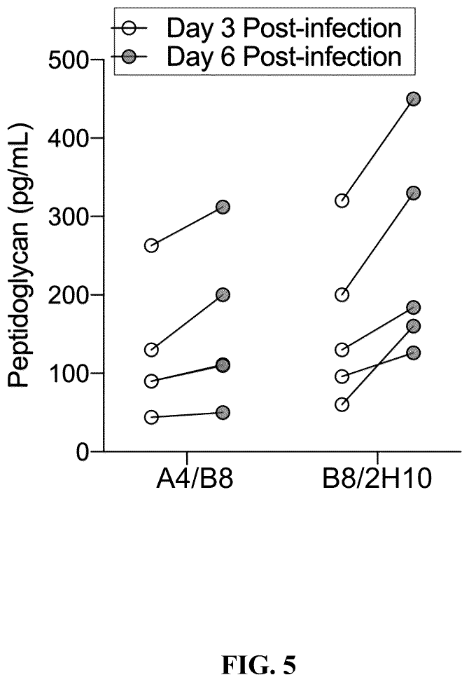

Animals were needle inoculated with B. burgdorferi and longitudinal serum samples were collected. A combination of 1A4, 1E10, 2G10, and 2H10 monoclonal antibodies were used to either capture antigen(s) in serum or detect the immobilized antigen(s) by ELISA (FIG. 5). These results indicate that a cocktail of all four antibodies is capable of detecting released peptidoglycan in Lyme disease-infected animals.

### Assay to Detect Peptidoglycan in Human Urine.

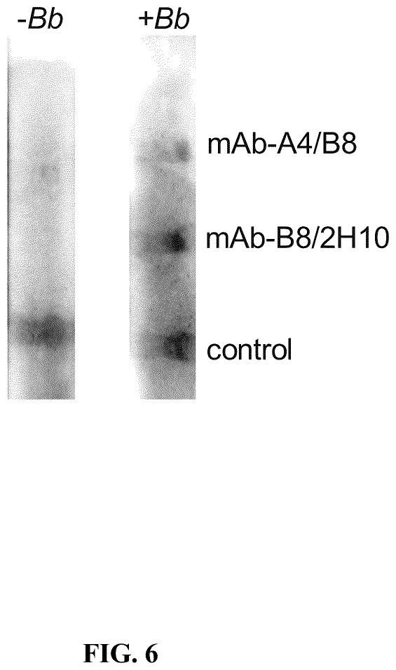

Nitrocellulose membranes were coated with a combination of A4, 1E10, 2G10, and 2H10 monoclonal antibodies or anti-human IgG (control). Pooled urine samples collected from human Lyme disease patients or uninfected, control individuals were incubated with coated membranes and detected using biotin-labeled antibodies using streptavidin: horseradish peroxidase by chemiluminescence (FIG. 6). This data at least suggests that it is possible to detect peptidoglycan in human urine.

### Example 2

Sequences related to the mAbs of Example 1. Recombinant polypeptide sequences of Table 1 were generated in silico from the polynucleotide sequences of Table 2.

TABLE 1
Recombinant mAb Polypeptide Sequences

| mAb | |
|---|---|
| Fragment | Amino Acid Sequence (5′ to 3′) |
| 1A4-H Heavy | METGLRWLLLVAVLKGVQCQSLEESGGRLVTPGTPLTLTCTVSGFSLS |
| Chain | SYAVIWVRQAPGEGLEYIGIIFGGGKTYYASWAKGRFTISKTSTTVDLR |
| | ITSPTTEDTATYFCVRDDWSRYLWGQGTLVTVSSGQPKAPSVFPLAPC |
| | CGDTPSSTVTLGCLVKGYLPEPVTVTWNSGTLINGVRTFPSVRQSSGL |
| | YSLSSVVSVTSSSQPVTCNVAHPATNTKVDKTVAPSTCSKPMCPPPELP |
| | GGPSVFIFPPKPKDTLMISRTPEVTCVVVDVSQDDPEVQFTWYINNEQ |
| | VRTARPPLREQQFNSTIRVVSTLPIAHQDWLRGKEFKCKVHNKALPAP |
| | IEKTISKARGQPLEPKVYTMGPPREELSSRSVSLTCMINGFYPSDISVEW |
| | EKNGKAEDNYKTTPTVLDSDGSYFLYSKLSVPTSEWQRGDVFTCSVM |
| | HEALHNHYTQKSISRSPGK |
| | (SEQ ID NO: 1) |
| 1A4-L Light | MDTRAPTQLLGLLLLWLPGATFAQVLTQTASPVSAAVGSTVTINCQSS |
| Chain | ESVYSNNYLSWFHQKPGQPPKQLIYSASTLASGVPSRFKGSGSGTQFT |
| | LTISDVQCDDAATYYCLGNYGCSSADCRAFGGGTEVVVKGDPVAPTV |
| | LIFPPAADQVATGTVTIVCVANKYFPDVTVTWEVDGTTQTTGIENSKT |
| | PQNSADCTYNLSSTLTLTSTQYNSHKEYTCKVTQGTTSVVQSFNRGDC |
| | (SEQ ID NO: 2) |
| 1E10-H Heavy | METGLRWLLLVAVLKGVQCQSVKESGGRLVTPGTPLTLTCTVSGMDL |
| Chain | SSNVVSWVRQAPGKGPEWIVTIGGSGRTFYATWAKGRFTVSKTSTSV |
| | DLKVTSLTTEDTATYFCARLWDIWGPGTLVTVSSGQPKAPSVFPLAPC |
| | CGDTPSSTVTLGCLVKGYLPEPVTVTWNSGTLTNGVRTFPSVRQSSGL |
| | YSLSSVVSVTSSSQPVTCNVAHPATNTKVDKTVAPSTCSKPMCPPPELP |
| | GGPSVFIFPPKPKDTLMISRTPEVTCVVVDVSQDDPEVQFTWYINNEQ |
| | VRTARPPLREQQFNSTIRVVSTLPIAHQDWLRGKEFKCKVHNKALPAP |
| | IEKTISKARGQPLEPKVYTMGPPREELSSRSVSLTCMINGFYPSDISVEW |
| | EKNGKAEDNYKTTPTVLDSDGSYFLYSKLSVPTSEWQRGDVFTCSVM |
| | HEALHNHYTQKSISRSPGK |
| | (SEQ ID NO: 3) |
| 1E10-L Light | MDTRAPTQLLGLLLLWLPGATFAQVLTQTPSSVSAAVGGTVTISCRSS |
| Chain | QSVYANNALSWYQQKPGQPPKLLIDSATTLPSGVPSRFRGFGSGTQFT |
| | LTISDLECDDAATYYCAGGYSGVIYVFGGGTEVVVKGDPVAPTVLIFP |
| | PAADQVATGTVTIVCVANKYFPDVTVTWEVDGTTQTTGIENSKTPQN |
| | SADCTYNLSSTLTLTSTQYNSHKEYTCKVTQGTTSVVQSFNRGDC |
| | (SEQ ID NO: 4) |
| 2H10-H | METGLRWLLLVAVLKGVQCQSVEESGGRLVTPGGSLTLTCTVSGFSL |
| Heavy Chain | SSYGMNWVRQAPGEGLKWIGTITTGGRAYYASWAKGRFTISRTSSTT |
| | VNLKMTSLTAEDTATYFCYRADYTTAIWGPGTLVTVSSGQPKAPSVFP |
| | LAPCCGDTPSSTVTLGCLVKGYLPEPVTVTWNSGTLINGVRTFPSVRQ |
| | SSGLYSLSSVVSVTSSSQPVTCNVAHPATNTKVDKTVAPSTCSKPMCP |
| | PPELPGGPSVFIFPPKPKDTLMISRTPEVTCVVVDVSQDDPEVQFTWYI |
| | NNEQVRTARPPLREQQFNSTIRVVSTLPIAHQDWLRGKEFKCKVHNK |
| | ALPAPIEKTISKARGQPLEPKVYTMGPPREELSSRSVSLTCMINGFYPSD |
| | ISVEWEKNGKAEDNYKTTPTVLDSDGSYFLYSKLSVPTSEWQRGDVF |
| | TCSVMHEALHNHYTQKSISRSPGK |
| | (SEQ ID NO: 5) |
| 2H10-L Light | MDTRAPTQLLGLLLLWLPGARCALVMTQTPSPVSAPVGGTVTISCQAS |
| Chain | QSVANNNDLAWFQQKPGQPPKLLIYGASTLASGVPSRFKGSGSGTQFT |
| | LTIDGVQCDDAASYYCVGYKSFSNDDFGFGGGTEVVVKGDPVAPTVL |
| | IFPPAADQVATGTVTIVCVANKYFPDVTVTWEVDGTTQTTGIENSKTP |
| | QNSADCTYNLSSTLTLTSTQYNSHKEYTCKVTQGTTSVVQSFNRGDC |
| | (SEQ ID NO: 6) |
| 2G10-H | METGLRWLLLVAVLKGVQCQSVEESGGRLVTPGTPLTLTCTVSGIDLS |
| Heavy Chain | RYAMSWVRQAPGEGLEYIGSISSGGITYYASWAKGRFTISKTSTTVDL |
| | KITSPTTEDTGTYFCGRVGTGDSVGIWGPGTLVTVSSGQPKAPSVFPLA |
| | PCCGDTPSSTVTLGCLVKGYLPEPVTVTWNSGTLTNGVRTFPSVRQSS |
| | GLYSLSSVVSVTSSSQPVTCNVAHPATNTKVDKTVAPSTCSKPMCPPP |
| | ELPGGPSVFIFPPKPKDTLMISRTPEVTCVVVDVSQDDPEVQFTWYINN |
| | EQVRTARPPLREQQFNSTIRVVSTLPIAHQDWLRGKEFKCKVHNKALP |
| | APIEKTISKARGQPLEPKVYTMGPPREELSSRSVSLTCMINGFYPSDISV |
| | EWEKNGKAEDNYKTTPTVLDSDGSYFLYSKLSVPTSEWQRGDVFTCS |
| | VMHEALHNHYTQKSISRSPGK |
| | (SEQ ID NO: 7) |
| 2G10-L Light | MDTRAPTQLLGLLLLWLPGATFAIVMTQTPSSKSVPVGGTVTINCQAS |
| Chain | QSVYKNNYLSWYQQKPGQPPKQLIYLASTLASGVPSRFKGSGSGTQFT |
| | LTITDVVCDDAATYYCVGWKTSNTDGYGFGGGTEVVVKGDPVAPTV |
| | LIFPPAADQVATGTVTIVCVANKYFPDVTVTWEVDGTTQTTGIENSKT |
| | PQNSADCTYNLSSTLTLTSTQYNSHKEYTCKVTQGTTSVVQSFNRGDC |
| | (SEQ ID NO: 8) |

TABLE 2
Recombinant mAb Nucleotide Sequences

| mAb Fragment | Nucleotide Sequence (5′ to 3′) |
|---|---|
| 1A4-H Heavy | ATGGAGACTGGGCTGCGCTGGCTTCTCCTGGTCGCTGTGCTCA |
| Chain | AAGGTGTCCAGTGTCAGTCGCTGGAGGAGTCCGGGGGTCGCCT |
| | GGTCACGCCTGGGACACCCCTGACACTCACCTGCACAGTCTCT |
| | GGATTCTCCCTCAGTAGCTATGCTGTGATCTGGGTCCGCCAGGC |
| | TCCAGGGGAGGGGCTGGAATACATCGGAATTATTTTTGGTGGT |
| | GGTAAGACATACTACGCGAGCTGGGCGAAAGGCCGATTCACC |
| | ATCTCCAAGACCTCGACCACGGTGGATCTGAGAATCACCAGTC |
| | CGACAACCGAGGACACGGCCACCTATTTCTGTGTCAGAGATGA |
| | TTGGAGTAGATATTTGTGGGGCCAAGGCACCCTGGTCACCGTC |
| | TCCTCAGGGCAACCTAAGGCTCCATCAGTCTTCCCACTGGCCCC |
| | CTGCTGCGGGGACACACCCAGCTCCACGGTGACCCTGGGCTGC |
| | CTGGTCAAAGGCTACCTCCCGGAGCCAGTGACCGTGACCTGGA |
| | ACTCGGGCACCCTCACCAATGGGGTACGCACCTTCCCGTCCGT |
| | CCGGCAGTCCTCAGGCCTCTACTCGCTGAGCAGCGTGGTGAGC |
| | GTGACCTCAAGCAGCCAGCCCGTCACCTGCAACGTGGCCCACC |
| | CAGCCACCAACACCAAAGTGGACAAGACCGTTGCGCCCTCGAC |
| | ATGCAGCAAGCCCATGTGCCCACCCCCTGAACTCCCGGGGGGA |
| | CCGTCTGTCTTCATCTTCCCCCCAAAACCCAAGGACACCCTCAT |
| | GATCTCACGCACCCCCGAGGTCACATGCGTGGTGGTGGACGTG |
| | AGCCAGGATGACCCCGAGGTGCAGTTCACATGGTACATAAACA |
| | ACGAGCAGGTGCGCACCGCCCGGCCGCCGCTACGGGAGCAGC |
| | AGTTCAACAGCACGATCCGCGTGGTCAGCACCCTCCCCATCGC |
| | GCACCAGGACTGGCTGAGGGGCAAGGAGTTCAAGTGCAAAGT |
| | CCACAACAAGGCACTCCCGGCCCCCATCGAGAAAACCATCTCC |
| | AAAGCCAGAGGGCAGCCCCTGGAGCCGAAGGTCTACACCATG |
| | GGCCCTCCCCGGGAGGAGCTGAGCAGCAGGTCGGTCAGCCTGA |
| | CCTGCATGATCAACGGCTTCTACCCTTCCGACATCTCGGTGGAG |
| | TGGGAGAAGAACGGGAAGGCAGAGGACAACTACAAGACCACG |
| | CCGACCGTGCTGGACAGCGACGGCTCCTACTTCCTCTACAGCA |
| | AGCTCTCAGTGCCCACGAGTGAGTGGCAGCGGGGCGACGTCTT |
| | CACCTGCTCCGTGATGCACGAGGCCTTGCACAACCACTACACG |
| | CAGAAGTCCATCTCCCGCTCTCCGGGTAAATAG |
| | (SEQ ID NO: 9) |
| 1A4-L Light Chain | ATGGACACGAGGGCCCCCACTCAGCTGCTGGGGCTCCTGCTGC |
| | TCTGGCTCCCAGGTGCCACATTTGCCCAAGTGCTGACCCAGAC |
| | TGCATCGCCCGTGTCTGCAGCTGTGGGAAGCACAGTCACCATC |
| | AATTGCCAGTCCAGTGAGAGTGTTTATAGTAACAACTACTTAT |
| | CCTGGTTTCACCAGAAACCAGGGCAGCCTCCCAAGCAACTGAT |
| | CTATTCTGCATCCACTCTGGCATCTGGGGTCCCATCGCGGTTCA |
| | AAGGCAGTGGATCTGGGACACAGTTCACTCTCACCATCAGCGA |
| | CGTGCAGTGTGACGATGCTGCCACTTACTACTGTCTGGGCAATT |
| | ATGGTTGTAGTAGTGCTGATTGTCGGGCTTTCGGCGGAGGGAC |
| | CGAGGTGGTGGTCAAAGGTGATCCAGTTGCACCTACTGTCCTC |
| | ATCTTCCCACCAGCTGCTGATCAGGTGGCAACTGGAACAGTCA |
| | CCATCGTGTGTGTGGCGAATAAATACTTTCCCGATGTCACCGTC |
| | ACCTGGGAGGTGGATGGCACCACCCAAACAACTGGCATCGAG |
| | AACAGTAAAACACCGCAGAATTCTGCAGATTGTACCTACAACC |
| | TCAGCAGCACTCTGACACTGACCAGCACACAGTACAACAGCCA |
| | CAAAGAGTACACCTGCAAGGTGACCCAGGGCACGACCTCAGTC |
| | GTCCAGAGCTTCAATAGGGGTGACTGTTAG |
| | (SEQ ID NO: 10) |
| 1E10-H Heavy | ATGGAGACTGGGCTGCGCTGGCTTCTCCTGGTCGCTGTGCTCA |
| Chain | AAGGTGTCCAGTGTCAGTCAGTGAAGGAGTCCGGGGGTCGCCT |
| | GGTCACGCCTGGGACACCCCTGACACTCACCTGCACAGTCTCT |
| | GGAATGGACCTCAGTAGTAATGTGGTGAGTTGGGTCCGCCAGG |
| | CTCCAGGGAAGGGGCCGGAATGGATCGTAACTATTGGTGGTAG |
| | TGGTAGGACATTTTACGCGACCTGGGCGAAAGGCCGATTCACC |
| | GTCTCCAAAACCTCGACCTCGGTGGATCTGAAAGTCACCAGTC |
| | TGACAACCGAGGACACGGCCACTTATTTCTGTGCCAGACTTTG |
| | GGACATCTGGGGCCCAGGCACCCTGGTCACCGTCTCCTCAGGG |
| | CAACCTAAGGCTCCATCAGTCTTCCCACTGGCCCCCTGCTGCGG |
| | GGACACACCCAGCTCCACGGTGACCCTGGGCTGCCTGGTCAAA |
| | GGCTACCTCCCGGAGCCAGTGACCGTGACCTGGAACTCGGGCA |
| | CCCTCACCAATGGGGTACGCACCTTCCCGTCCGTCCGGCAGTC |
| | CTCAGGCCTCTACTCGCTGAGCAGCGTGGTGAGCGTGACCTCA |
| | AGCAGCCAGCCCGTCACCTGCAACGTGGCCCACCCAGCCACCA |
| | ACACCAAAGTGGACAAGACCGTTGCGCCCTCGACATGCAGCAA |
| | GCCCATGTGCCCACCCCCTGAACTCCCGGGGGGACCGTCTGTC |
| | TTCATCTTCCCCCCAAAACCCAAGGACACCCTCATGATCTCACG |
| | CACCCCCGAGGTCACATGCGTGGTGGTGGACGTGAGCCAGGAT |
| | GACCCCGAGGTGCAGTTCACATGGTACATAAACAACGAGCAG |
| | GTGCGCACCGCCCGGCCGCCGCTACGGGAGCAGCAGTTCAACA |
| | GCACGATCCGCGTGGTCAGCACCCTCCCCATCGCGCACCAGGA |
| | CTGGCTGAGGGGCAAGGAGTTCAAGTGCAAAGTCCACAACAA |
| | GGCACTCCCGGCCCCCATCGAGAAAACCATCTCCAAAGCCAGA |
| | GGGCAGCCCCTGGAGCCGAAGGTCTACACCATGGGCCCTCCCC |
| | GGGAGGAGCTGAGCAGCAGGTCGGTCAGCCTGACCTGCATGAT |
| | CAACGGCTTCTACCCTTCCGACATCTCGGTGGAGTGGGAGAAG |
| | AACGGGAAGGCAGAGGACAACTACAAGACCACGCCGACCGTG |
| | CTGGACAGCGACGGCTCCTACTTCCTCTACAGCAAGCTCTCAG |
| | TGCCCACGAGTGAGTGGCAGCGGGGCGACGTCTTCACCTGCTC |
| | CGTGATGCACGAGGCCTTGCACAACCACTACACGCAGAAGTCC |
| | ATCTCCCGCTCTCCGGGTAAATAG |
| | (SEQ ID NO: 11) |
| 1E10-L Light | ATGGACACGAGGGCCCCCACTCAGCTGCTGGGGCTCCTGCTGC |
| Chain | TCTGGCTCCCAGGTGCCACATTCGCGCAAGTGCTGACCCAGAC |
| | TCCATCCTCCGTGTCTGCAGCTGTGGGAGGCACAGTCACCATA |
| | AGTTGCCGGTCCAGTCAGAGTGTTTATGCGAACAACGCCTTAT |
| | CCTGGTATCAGCAGAAACCAGGGCAGCCTCCCAAGCTCCTGAT |
| | CGATTCTGCTACCACTCTGCCATCTGGGGTCCCATCGCGATTCA |
| | GAGGCTTCGGATCTGGGACACAGTTCACTCTCACCATCAGCGA |
| | CCTGGAGTGTGACGATGCTGCCACTTACTACTGTGCAGGCGGT |
| | TATAGTGGTGTCATTTATGTTTTCGGCGGAGGGACCGAGGTGG |
| | TGGTCAAAGGTGATCCAGTTGCACCTACTGTCCTCATCTTCCCA |
| | CCAGCTGCTGATCAGGTGGCAACTGGAACAGTCACCATCGTGT |
| | GTGTGGCGAATAAATACTTTCCCGATGTCACCGTCACCTGGGA |
| | GGTGGATGGCACCACCCAAACAACTGGCATCGAGAACAGTAA |
| | AACACCGCAGAATTCTGCAGATTGTACCTACAACCTCAGCAGC |
| | ACTCTGACACTGACCAGCACACAGTACAACAGCCACAAAGAGT |
| | ACACCTGCAAGGTGACCCAGGGCACGACCTCAGTCGTCCAGAG |
| | CTTCAATAGGGGTGACTGTTAG |
| | (SEQ ID NO: 12) |
| 2H10-H Heavy | ATGGAGACTGGGCTGCGCTGGCTTCTCCTGGTCGCTGTGCTCA |
| Chain | AAGGTGTCCAGTGTCAGTCGGTGGAGGAGTCCGGGGGTCGCCT |
| | GGTCACGCCTGGAGGATCCCTGACACTCACCTGTACCGTCTCT |
| | GGATTCTCCCTCAGTAGCTATGGAATGAACTGGGTCCGCCAGG |
| | CTCCAGGTGAGGGACTGAAATGGATCGGAACCATTACTACTGG |
| | TGGTCGCGCATATTACGCGAGCTGGGCGAAAGGCCGATTCACC |
| | ATCTCCAGAACCTCGTCGACCACGGTGAATCTGAAAATGACCA |
| | GTCTGACAGCCGAGGACACGGCCACCTATTTTTGTTACAGAGC |
| | TGATTACACTACTGCCATCTGGGGCCCAGGCACCCTGGTCACC |
| | GTCTCCTCAGGGCAACCTAAGGCTCCATCAGTCTTCCCACTGGC |
| | CCCCTGCTGCGGGGACACACCCAGCTCCACGGTGACCCTGGGC |
| | TGCCTGGTCAAAGGCTACCTCCCGGAGCCAGTGACCGTGACCT |
| | GGAACTCGGGCACCCTCACCAATGGGGTACGCACCTTCCCGTC |
| | CGTCCGGCAGTCCTCAGGCCTCTACTCGCTGAGCAGCGTGGTG |
| | AGCGTGACCTCAAGCAGCCAGCCCGTCACCTGCAACGTGGCCC |
| | ACCCAGCCACCAACACCAAAGTGGACAAGACCGTTGCGCCCTC |
| | GACATGCAGCAAGCCCATGTGCCCACCCCCTGAACTCCCGGGG |
| | GGACCGTCTGTCTTCATCTTCCCCCCAAAACCCAAGGACACCCT |
| | CATGATCTCACGCACCCCCGAGGTCACATGCGTGGTGGTGGAC |
| | GTGAGCCAGGATGACCCCGAGGTGCAGTTCACATGGTACATAA |
| | ACAACGAGCAGGTGCGCACCGCCCGGCCGCCGCTACGGGAGC |
| | AGCAGTTCAACAGCACGATCCGCGTGGTCAGCACCCTCCCCAT |
| | CGCGCACCAGGACTGGCTGAGGGGCAAGGAGTTCAAGTGCAA |
| | AGTCCACAACAAGGCACTCCCGGCCCCCATCGAGAAAACCATC |
| | TCCAAAGCCAGAGGGCAGCCCCTGGAGCCGAAGGTCTACACC |
| | ATGGGCCCTCCCCGGGAGGAGCTGAGCAGCAGGTCGGTCAGCC |
| | TGACCTGCATGATCAACGGCTTCTACCCTTCCGACATCTCGGTG |
| | GAGTGGGAGAAGAACGGGAAGGCAGAGGACAACTACAAGACC |
| | ACGCCGACCGTGCTGGACAGCGACGGCTCCTACTTCCTCTACA |
| | GCAAGCTCTCAGTGCCCACGAGTGAGTGGCAGCGGGGCGACGT |
| | CTTCACCTGCTCCGTGATGCACGAGGCCTTGCACAACCACTAC |
| | ACGCAGAAGTCCATCTCCCGCTCTCCGGGTAAATAG |
| | (SEQ ID NO: 13) |
| 2H10-L Light | ATGGACACGAGGGCCCCCACTCAGCTGCTGGGGCTCCTGCTGC |
| Chain | TCTGGCTCCCAGGTGCCAGATGTGCCCTTGTGATGACCCAGAC |
| | TCCATCCCCCGTGTCTGCACCTGTGGGAGGCACAGTCACCATC |
| | AGTTGCCAGGCCAGTCAGAGTGTTGCTAATAACAACGACTTAG |
| | CCTGGTTTCAGCAGAAACCAGGGCAGCCTCCCAAGCTCCTGAT |
| | CTATGGTGCATCCACTCTGGCATCTGGGGTCCCATCGCGGTTCA |
| | AAGGCAGTGGATCTGGGACACAGTTCACTCTCACCATCGACGG |
| | CGTGCAGTGTGACGATGCTGCCAGTTACTATTGTGTAGGCTAT |
| | AAAAGTTTTAGTAATGATGATTTTGGTTTCGGCGGAGGGACCG |
| | AGGTGGTGGTCAAAGGTGATCCAGTTGCACCTACTGTCCTCAT |
| | CTTCCCACCAGCTGCTGATCAGGTGGCAACTGGAACAGTCACC |
| | ATCGTGTGTGTGGCGAATAAATACTTTCCCGATGTCACCGTCAC |
| | CTGGGAGGTGGATGGCACCACCCAAACAACTGGCATCGAGAA |
| | CAGTAAAACACCGCAGAATTCTGCAGATTGTACCTACAACCTC |
| | AGCAGCACTCTGACACTGACCAGCACACAGTACAACAGCCACA |
| | AAGAGTACACCTGCAAGGTGACCCAGGGCACGACCTCAGTCGT |
| | CCAGAGCTTCAATAGGGGTGACTGTTAG |
| | (SEQ ID NO: 14) |
| 2G10-H Heavy | ATGGAGACTGGGCTGCGCTGGCTTCTCCTGGTCGCTGTGCTCA |
| Chain | AAGGTGTCCAGTGTCAGTCGGTGGAGGAGTCCGGGGGTCGCCT |
| | GGTCACGCCTGGGACACCCCTGACACTCACCTGCACAGTCTCT |
| | GGAATCGACCTCAGTAGGTATGCAATGAGCTGGGTCCGCCAGG |
| | CTCCAGGGGAGGGCCTGGAATACATCGGATCCATTAGTAGTGG |
| | TGGTATCACATATTACGCGAGCTGGGCGAAAGGCCGATTCACC |
| | ATCTCCAAAACCTCGACCACGGTGGATCTCAAAATCACCAGTC |
| | CGACAACCGAGGACACGGGCACCTATTTCTGTGGCAGAGTCGG |
| | AACTGGTGATAGTGTTGGCATCTGGGGCCCAGGCACCCTGGTC |
| | ACCGTCTCCTCAGGGCAACCTAAGGCTCCATCAGTCTTCCCACT |
| | GGCCCCCTGCTGCGGGGACACACCCAGCTCCACGGTGACCCTG |
| | GGCTGCCTGGTCAAAGGCTACCTCCCGGAGCCAGTGACCGTGA |
| | CCTGGAACTCGGGCACCCTCACCAATGGGGTACGCACCTTCCC |
| | GTCCGTCCGGCAGTCCTCAGGCCTCTACTCGCTGAGCAGCGTG |
| | GTGAGCGTGACCTCAAGCAGCCAGCCCGTCACCTGCAACGTGG |
| | CCCACCCAGCCACCAACACCAAAGTGGACAAGACCGTTGCGCC |
| | CTCGACATGCAGCAAGCCCATGTGCCCACCCCCTGAACTCCCG |
| | GGGGGACCGTCTGTCTTCATCTTCCCCCCAAAACCCAAGGACA |
| | CCCTCATGATCTCACGCACCCCCGAGGTCACATGCGTGGTGGT |
| | GGACGTGAGCCAGGATGACCCCGAGGTGCAGTTCACATGGTAC |
| | ATAAACAACGAGCAGGTGCGCACCGCCCGGCCGCCGCTACGG |
| | GAGCAGCAGTTCAACAGCACGATCCGCGTGGTCAGCACCCTCC |
| | CCATCGCGCACCAGGACTGGCTGAGGGGCAAGGAGTTCAAGT |
| | GCAAAGTCCACAACAAGGCACTCCCGGCCCCCATCGAGAAAA |
| | CCATCTCCAAAGCCAGAGGGCAGCCCCTGGAGCCGAAGGTCTA |
| | CACCATGGGCCCTCCCCGGGAGGAGCTGAGCAGCAGGTCGGTC |
| | AGCCTGACCTGCATGATCAACGGCTTCTACCCTTCCGACATCTC |
| | GGTGGAGTGGGAGAAGAACGGGAAGGCAGAGGACAACTACAA |
| | GACCACGCCGACCGTGCTGGACAGCGACGGCTCCTACTTCCTC |
| | TACAGCAAGCTCTCAGTGCCCACGAGTGAGTGGCAGCGGGGCG |
| | ACGTCTTCACCTGCTCCGTGATGCACGAGGCCTTGCACAACCA |
| | CTACACGCAGAAGTCCATCTCCCGCTCTCCGGGTAAATAG |
| | (SEQ ID NO: 15) |
| 2G10-L Light | ATGGACACGAGGGCCCCCACTCAGCTGCTGGGGCTCCTGCTGC |
| Chain | TCTGGCTCCCAGGTGCCACATTTGCCATCGTGATGACCCAGACT |
| | CCATCTTCCAAATCTGTCCCTGTGGGAGGCACAGTCACCATCA |
| | ATTGCCAGGCCAGTCAGAGTGTTTATAAGAACAACTACTTATC |
| | CTGGTATCAGCAGAAACCAGGGCAGCCTCCCAAGCAACTGATC |
| | TATCTGGCATCCACTCTGGCATCTGGGGTCCCATCGCGGTTCAA |
| | AGGCAGTGGATCTGGGACACAGTTCACTCTCACCATCACCGAT |
| | GTGGTGTGTGACGATGCTGCCACTTACTACTGTGTAGGATGGA |
| | AAACTAGTAATACTGATGGGTATGGTTTCGGCGGAGGGACCGA |
| | GGTGGTGGTCAAAGGTGATCCAGTTGCACCTACTGTCCTCATCT |
| | TCCCACCAGCTGCTGATCAGGTGGCAACTGGAACAGTCACCAT |
| | CGTGTGTGTGGCGAATAAATACTTTCCCGATGTCACCGTCACCT |
| | GGGAGGTGGATGGCACCACCCAAACAACTGGCATCGAGAACA |
| | GTAAAACACCGCAGAATTCTGCAGATTGTACCTACAACCTCAG |
| | CAGCACTCTGACACTGACCAGCACACAGTACAACAGCCACAAA |
| | GAGTACACCTGCAAGGTGACCCAGGGCACGACCTCAGTCGTCC |
| | AGAGCTTCAATAGGGGTGACTGTTAG |
| | (SEQ ID NO: 16) |

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

Further attributes, features, and embodiments of the present invention can be understood by reference to the following numbered aspects of the disclosed invention. Reference to disclosure in any of the preceding aspects is applicable to any preceding numbered aspect and to any combination of any number of preceding aspects, as recognized by appropriate antecedent disclosure in any combination of preceding aspects that can be made. The following numbered aspects are provided:
1. An antibody or fragment there of capable of specifically binding a Borreliae peptidoglycan.
2. The The antibody or fragment thereof of aspect 1, wherein the Borreliae peptidoglycan is a Borrelia burgdorferi, Borrelia afzelii, Borrelia garinii, Borrelia japonica, Borrelia andersonii, Borrelia bissettii, Borrelia sinica, Borrelia turdi, Borrelia tanukii, Borrelia valaisiana, Borrelia lusitaniae, Borrelia spielmanii, Borrelia miyamotoi, or a Borrelia Lonestar peptidoglycan.
3. The antibody or fragment thereof of any one of aspects 1-2, wherein the antibody or fragment thereof is capable of binding (a) a glycan component of the Borreliae peptidoglycan, optionally a GlcNAc-GlcNAc-anhMurNAc, (b) an alpha-Orn linkage in a Borreliae peptidoglycan, (c) a gamma-Orn linkage in a Borreliae peptidoglycan, or (d) a Borreliae muropeptide comprising AnuMurNAc.
4. The antibody or fragment thereof of any one of aspects 1-3, wherein the antibody or fragment thereof comprises one or more polypeptides each independently having a sequence that is 80-100 percent identical to one or more of SEQ ID NO: 1-8.
5. The antibody or fragment thereof of any one of aspects 1 to 4, wherein the antibody is a chimeric antibody, a human antibody, or a humanized antibody.
6. The antibody or fragment thereof of any one of aspects 1 to 5, wherein the antibody or fragment thereof comprises a Fab, a Fab′, a F(ab′)2, a Fv, a single chain Fv (scFv), or combinations thereof.
7. The antibody or fragment thereof of any one of aspects 1 to 6, wherein the antibody or fragment thereof is selected from the group consisting of an IgG1, an IgG2, an IgG3, an IgG4, a variant thereof, and any combination thereof.
8. The antibody or fragment thereof of any one of aspects 1 to 7, wherein the antibody or fragment thereof is linked to an agent, thereby forming an immunoconjugate.
9. The antibody or fragment thereof of any one of the aspects 1-8, wherein the antibody or fragment thereof is a monoclonal antibody or fragment thereof.
10. The antibody or fragment thereof of any one of aspects 1-9, wherein the monoclonal antibody or fragment thereof is selected from 1A4-1, 1E10, 1G11, or 2H10.
11. A polynucleotide encoding the antibody or fragment thereof of any one of aspects 1-10.
12. The polynucleotide of aspect 11, wherein the polynucleotide comprises or consists of a sequence that is 70-100 percent identical to any one of SEQ ID NOs: 9-16.
13. A vector comprising: the polynucleotide of any one of aspects 11-12.
14. A cell or population thereof comprising: (a) the antibody or fragment thereof of any one of aspects 1-10; (b) the polynucleotide of any one of aspects 11-12; (c) the vector of aspect 13; or (d) any combination of (a)-(c).
15. A pharmaceutical formulation comprising: (a) the antibody or fragment thereof of any one of aspects 1-10; (b) the polynucleotide of any one of aspects 11-12; (c) the vector of aspect 13; (d) the cell or population thereof of aspect 14, or (e) any combination of (a)-(d); and a pharmaceutically acceptable carrier.
16. The pharmaceutical formulation of aspect 15, further comprising a co-therapeutic agent, wherein the co-therapeutic agent is effective to treat Lyme disease or a symptom thereof.
17. A kit comprising: (a) the antibody or fragment thereof of any one of aspects 1-10; (b) the polynucleotide of any one of aspects 11-12; (c) the vector of aspect 13; (d) the cell or population thereof of aspect 14; (e) the pharmaceutical formulation of any one of aspects 15-16; or (f) any combination of (a)-(e).
18. A method of detecting and/or quantifying a Borreliae peptidoglycan, and/or diagnosing a Borreliae infection, and/or Lyme disease in a subject in need thereof, the method comprising: contacting the antibody or fragment thereof of any one of aspects 1-10 with a biological sample from the subject in need thereof and detecting and/or quantifying the amount of Borreliae peptidoglycan in the sample.
19. The method of aspect 18, wherein the Borreliae peptidoglycan is a Borrelia burgdorferi, Borrelia afzelii, Borrelia garinii, Borrelia japonica, Borrelia andersonii, Borrelia bissettii, Borrelia sinica, Borrelia turdi, Borrelia tanukii, Borrelia valaisiana, Borrelia lusitaniae, Borrelia spielmanii, Borrelia miyamotoi, or a Borrelia Lonestar peptidoglycan.
20. The method of any one of aspects 18-19, wherein the biological sample of the subject has a greater amount of Borreliae peptidoglycan as compared to a reference or a control sample.
21. The method of any one of aspects 18-20, wherein the level of Borreliae peptidoglycan in the biological sample of the subject is greater than at least about 5%, at least about 10%, at least about 20%, at least about 30%, at least about 40%, at least about 50%, at least about 60%, at least about 70%, at least about 80%, at least about 90%, at least about 100%, at least about 150%, at least about 200%, at least about 300% or more compared to a corresponding level in the reference or control sample.
22. The method of any one of aspects 18-21, wherein the contacting, detecting and/or quantifying is performed in vitro.
23. The method of any one of aspects 18-22, wherein the biological sample comprises synovial fluid, serum, or urine.
24. The method of any one of aspects 18-23, further comprising treating the subject with one or more therapeutic agents.
25. The method of aspect 24, wherein the one or more therapeutic agents comprise (a) an antibody or fragment thereof of any one of aspects 1-10; (b) the polynucleotide of any one of aspects 11-12; (c) the vector of aspect 13; (c) the cell or population thereof of aspect 14; (d) the pharmaceutical formulation of any one of aspects 15-16; (e) a therapeutic agent effective to treat Lyme disease or a symptom thereof; or (f) any combination of (a)-(e).
26. The method of any of aspects 24-25, wherein the one or more therapeutic agents comprise an antibody or fragment thereof of any one of aspects 1-10, an antibiotic, a peptidoglycan-specific hydrolase, or any combination thereof.
27. The method of aspect 26, wherein the antibiotic comprises doxycycline, amoxicillin, ceftriaxone, or any combination thereof.
28. The method of any one of aspects 24-27, wherein treating occurs after detecting and/or diagnosing.
29. The method of any one of aspects 18-28, wherein the contacting is performed within about 3 weeks after suspected infection with Borreliae organism.
30. The method of aspect 29, wherein the Borreliae organism is Borrelia burgdorferi, Borrelia afzelii, Borrelia garinii, Borrelia japonica, Borrelia andersonii, Borrelia bissettii, Borrelia sinica, Borrelia turdi, Borrelia tanukii, Borrelia valaisiana, Borrelia lusitaniae, Borrelia spielmanii, Borrelia miyamotoi, or Borrelia Lonestar.
31. A method of treating a Borreliae peptidoglycan disease or symptom thereof, a Borreliae infection or a symptom thereof, and/or Lyme disease or a symptom thereof in a subject in need thereof, the method comprising: administering (a) an antibody or fragment thereof of any one of aspects 1-10; (b) the polynucleotide of any one of aspects 11-12; (c) the vector of aspect 13; (c) the cell or population thereof of aspect 14; (d) the pharmaceutical formulation of any one of aspects 15-16; or (e) any combination of (a)-(d) to the subject in need thereof.
32. A method of reducing, inhibiting, or ameliorating one or more symptoms of Lyme and/or Borreliae disease in a subject in need thereof comprising administering to the subject an amount of (a) an antibody or fragment thereof of any one of aspects 1-10; (b) the polynucleotide of any one of aspects 11-12; (c) the vector of aspect 13; (c) the cell or population thereof of aspect 14; (d) the pharmaceutical formulation of any one of aspects 15-16; or (e) any combination of (a)-(d).
33. The method of any one of aspects 31-32, wherein administering reduces the level of one or more pro-inflammatory mediators in the subject compared to a reference or a control.
34. The method of aspect 33, wherein the one or more pro-inflammatory mediators comprise TNF-α, IL-1α, IL-1β, IL-6, IL-8, IFN-γ, IL-17F, or combinations thereof.
35. The method of any one of aspects 33-34, wherein the level of one or more pro-inflammatory mediators is reduced by at least about 5%, at least about 10%, at least about 20%, at least about 30%, at least about 40%, at least about 50%, at least about 60%, at least about 70%, at least about 80%, at least about 90% or more compared to the reference.
36. The method of any one of aspects 31-35, wherein the subject is administered an additional therapeutic agent.
37. The method of aspect 36, wherein the additional therapeutic agent comprises an antibiotic or a peptidoglycan-specific hydrolase.
38. The method of aspect 37, (a) wherein the antibiotic comprises doxycycline, amoxicillin, ceftriaxone, or combinations thereof, (b) wherein the peptidoglycan-specific hydrolase comprises an amidase or a lysozyme, or (c) both (a) and (b).
39. The method of any one of aspects 31-38, wherein the subject has or is suspected of having Lyme arthritis, neuroborreliosis or other Lyme disease-related clinical manifestations.

1. An antibody or fragment thereof capable of specifically binding a Borreliae peptidoglycan.

2. The antibody or fragment thereof of claim 1, wherein the Borreliae peptidoglycan is a Borrelia burgdorferi, Borrelia afzelii, Borrelia garinii, Borrelia japonica, Borrelia andersonii, Borrelia bissettii, Borrelia sinica, Borrelia turdi, Borrelia tanukii, Borrelia valaisiana, Borrelia lusitaniae, Borrelia spielmanii, Borrelia miyamotoi, or a Borrelia Lonestar peptidoglycan.

3. The antibody or fragment thereof of claim 1, wherein the antibody or fragment thereof is capable of binding (a) a glycan component of the Borreliae peptidoglycan, optionally a GlcNAc-GlcNAc-anhMurNAc, (b) an alpha-Orn linkage in a Borreliae peptidoglycan, (c) a gamma-Orn linkage in a Borreliae peptidoglycan, or (d) a Borreliae muropeptide comprising AnuMurNAc.

4. The antibody or fragment thereof of claim 1, wherein the antibody or fragment thereof comprises one or more polypeptides each independently having a sequence that is 80-100 percent identical to one or more of SEQ ID NO: 1-8.

5. The antibody or fragment thereof of claim 1, wherein the antibody is a chimeric antibody, a human antibody, or a humanized antibody.

6. The antibody or fragment thereof of claim 1, wherein the antibody or fragment thereof comprises a Fab, a Fab′, a F(ab′)2, a Fv, a single chain Fv (scFv), or combinations thereof.

7. The antibody or fragment thereof of claim 1, wherein the antibody or fragment thereof is selected from the group consisting of: an IgG1, an IgG2, an IgG3, an IgG4, a variant thereof, and any combination thereof.

8. The antibody or fragment thereof of claim 1, wherein the antibody or fragment thereof is linked to an agent, thereby forming an immunoconjugate.

9. The antibody or fragment thereof of claim 1, wherein the antibody or fragment thereof is a monoclonal antibody or fragment thereof.

10. The antibody or fragment thereof of claim 9, wherein the monoclonal antibody is selected from 1A4-1, 1E10, 1G11, or 2H10.

11. A polynucleotide encoding the antibody or fragment thereof of any one of claim 1.

12. The polynucleotide of claim 11, wherein the polynucleotide comprises or consists of a sequence that is 70-100 percent identical to any one of SEQ ID NOs: 9-16.

13. A vector comprising:
the polynucleotide of any one of claims 11-12.

14. A cell or population thereof comprising:
a. the antibody or fragment thereof of any one of claims 1-10;
b. the polynucleotide of any one of claims 11-12;
c. the vector of claim 13; or
d. any combination of (a)-(c).

15. A pharmaceutical formulation comprising:
a. the antibody or fragment thereof of any one of claims 1-10;
b. the polynucleotide of any one of claims 11-12;
c. the vector of claim 13; or
d. the cell or population thereof of claim 14; or
e. any combination of (a)-(d); and
a pharmaceutically acceptable carrier.

16. The pharmaceutical formulation of claim 15, further comprising a co-therapeutic agent, wherein the co-therapeutic agent is effective to treat Lyme's disease or a symptom thereof.

17. A kit comprising:
a. the antibody or fragment thereof of any one of claims 1-10;
b. the polynucleotide of any one of claims 11-12;
c. the vector of claim 13; or
d. the cell or population thereof of claim 14;
e. the pharmaceutical formulation of any one of claims 15-16; or
f. any combination of (a)-(e).

18. A method of detecting and/or quantifying a Borreliae peptidoglycan, and/or diagnosing a Borreliae infection, and/or Lyme's disease in a subject in need thereof, the method comprising:
contacting the antibody or fragment thereof of any one of claims 1-10 with a biological sample from the subject in need thereof and detecting and/or quantifying the amount of Borreliae peptidoglycan in the sample.

19. The method of claim 18, wherein the Borreliae peptidoglycan is a Borrelia burgdorferi, Borrelia afzelii, Borrelia garinii, Borrelia japonica, Borrelia andersonii, Borrelia bissettii, Borrelia sinica, Borrelia turdi, Borrelia tanukii, Borrelia valaisiana, Borrelia lusitaniae, Borrelia spielmanii, Borrelia miyamotoi, or a Borrelia Lonestar peptidoglycan.

20. The method of claim 18, wherein the biological sample of the subject has a greater amount of Borreliae peptidoglycan as compared to a reference or control sample.

21. The method of claim 18, wherein the level of Borreliae peptidoglycan in the biological sample of the subject is greater than at least about 5%, at least about 10%, at least about 20%, at least about 30%, at least about 40%, at least about 50%, at least about 60%, at least about 70%, at least about 80%, at least about 90%, at least about 100%, at least about 150%, at least about 200%, at least about 300% or more compared to a corresponding level in the reference or control sample.

22. The method of claim 18, wherein the contacting and detecting and/or quantifying is performed in vitro.

23. The method of claim 18, wherein the biological sample comprises synovial fluid, serum, or urine.

24. The method of claim 18, further comprising treating the subject with one or more therapeutic agents.

25. The method of claim 24, wherein the one or more therapeutic agents comprise (a) an antibody or fragment thereof of any one of claims 1-10; (b) the polynucleotide of any one of claims 11-12; (c) the vector of claim 13; (c) the cell or population thereof of claim 14; (d) the pharmaceutical formulation of any one of claims 15-16; (e) a therapeutic agent effective to treat Lyme's disease or a symptom thereof; or (f) any combination of (a)-(e).

26. The method of any of claims 24-25, wherein the one or more therapeutic agents comprise an antibody of any one of claims 1-10, an antibiotic, a peptidoglycan-specific hydrolase, or any combination thereof.

27. The method of claim 26, wherein the antibiotic comprises doxycycline, amoxicillin, ceftriaxone, or any combination thereof.

28. The method of any of claims 24-27, wherein treating occurs after detecting and/or diagnosing.

29. The method of any one of claims 18-28, wherein the contacting is performed within about 3 weeks after suspected infection with a Borreliae organism.

30. The method of claim 29, wherein the Borreliae organism is Borrelia burgdorferi, Borrelia afzelii, Borrelia garinii, Borrelia japonica, Borrelia andersonii, Borrelia bissettii, Borrelia sinica, Borrelia turdi, Borrelia tanukii, Borrelia valaisiana, Borrelia lusitaniae, Borrelia spielmanii, Borrelia miyamotoi, or Borrelia Lonestar.

31. A method of treating a Borreliae peptidoglycan disease or symptom thereof, a Borreliae infection or a symptom thereof, and/or Lyme's disease or a symptom thereof in a subject in need thereof, the method comprising:
administering (a) an antibody or fragment thereof of any one of claims 1-10; (b) the polynucleotide of any one of claims 11-12; (c) the vector of claim 13; (c) the cell or population thereof of claim 14; (d) the pharmaceutical formulation of any one of claims 15-16; or (e) any combination of (a)-(d) to the subject in need thereof.

32. A method of reducing, inhibiting, or ameliorating one or more symptoms of Lyme's and/or Borrelia peptidoglycan disease in a subject in need thereof comprising administering to the subject an amount (a) an antibody or fragment thereof of any one of claims 1-10; (b) the polynucleotide of any one of claims 11-12; (c) the vector of claim 13; (c) the cell or population thereof of claim 14; (d) the pharmaceutical formulation of any one of claims 15-16; or (e) any combination of (a)-(d).

33. The method of any one of claims 31-32, wherein administering reduces the level of one or more pro-inflammatory mediators in the subject compared to a reference or a control.

34. The method of claim 33, wherein the one or more pro-inflammatory mediators comprise TNF-α, IL-1α, IL-1β, IL-6, IL-8, IFN-γ, IL-17F, or combinations thereof.

35. The method of any one of claims 33-34, wherein the level of one or more pro-inflammatory mediators is reduced by at least about 5%, at least about 10%, at least about 20%, at least about 30%, at least about 40%, at least about 50%, at least about 60%, at least about 70%, at least about 80%, at least about 90% or more compared to the reference or the control.

36. The method of any one of claims 31-35, wherein the subject is administered an additional therapeutic agent.

37. The method of claim 36, wherein the additional therapeutic agent comprises an antibiotic or a peptidoglycan-specific hydrolase.

38. The method of claim 37, (a) wherein the antibiotic comprises doxycycline, amoxicillin, ceftriaxone, or combinations thereof, (b) wherein the peptidoglycan-specific hydrolase comprises an amidase or a lysozyme, or (c) both (a) and (b).

39. The method of any one of claims 31-38, wherein the subject has or is suspected of having Lyme arthritis, neuroborreliosis or other Lyme disease-related clinical manifestations.


SEQUENCE LISTING (20 sequences)

SEQ ID NO: 1 - protein, 454 aa, synthetic construct
  source 1..454: note = 1A4-H Heavy Chain
METGLRWLLLVAVLKGVQCQSLEESGGRLVTPGTPLTLTCTVSGFSLSSYAVIWVRQAPGEGLEYIGIIFGGGKTYYASWAKGRFTISKTSTTVDLRITSPTTEDTATYFCVRDDWSRYLWGQGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPMCPPPELPGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPTVLDSDGSYFLYSKLSVPTSEWQRGDVFTCSVMHEALHNHYTQKSISRSPGK

SEQ ID NO: 2 - protein, 239 aa, synthetic construct
  source 1..239: note = 1A4-L Light Chain
MDTRAPTQLLGLLLLWLPGATFAQVLTQTASPVSAAVGSTVTINCQSSESVYSNNYLSWFHQKPGQPPKQLIYSASTLASGVPSRFKGSGSGTQFTLTISDVQCDDAATYYCLGNYGCSSADCRAFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 3 - protein, 451 aa, synthetic construct
  source 1..451: note = 1E10-H Heavy Chain
METGLRWLLLVAVLKGVQCQSVKESGGRLVTPGTPLTLTCTVSGMDLSSNVVSWVRQAPGKGPEWIVTIGGSGRTFYATWAKGRFTVSKTSTSVDLKVTSLTTEDTATYFCARLWDIWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPMCPPPELPGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPTVLDSDGSYFLYSKLSVPTSEWQRGDVFTCSVMHEALHNHYTQKSISRSPGK

SEQ ID NO: 4 - protein, 236 aa, synthetic construct
  source 1..236: note = 1E10-L Light Chain
MDTRAPTQLLGLLLLWLPGATFAQVLTQTPSSVSAAVGGTVTISCRSSQSVYANNALSWYQQKPGQPPKLLIDSATTLPSGVPSRFRGFGSGTQFTLTISDLECDDAATYYCAGGYSGVIYVFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 5 - protein, 455 aa, synthetic construct
  source 1..455: note = 2H10-H Heavy Chain
METGLRWLLLVAVLKGVQCQSVEESGGRLVTPGGSLTLTCTVSGFSLSSYGMNWVRQAPGEGLKWIGTITTGGRAYYASWAKGRFTISRTSSTTVNLKMTSLTAEDTATYFCYRADYTTAIWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPMCPPPELPGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPTVLDSDGSYFLYSKLSVPTSEWQRGDVFTCSVMHEALHNHYTQKSISRSPGK

SEQ ID NO: 6 - protein, 238 aa, synthetic construct
  source 1..238: note = 2H10-L Light Chain
MDTRAPTQLLGLLLLWLPGARCALVMTQTPSPVSAPVGGTVTISCQASQSVANNNDLAWFQQKPGQPPKLLIYGASTLASGVPSRFKGSGSGTQFTLTIDGVQCDDAASYYCVGYKSFSNDDFGFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 7 - protein, 456 aa, synthetic construct
  source 1..456: note = 2G10-H Heavy Chain
METGLRWLLLVAVLKGVQCQSVEESGGRLVTPGTPLTLTCTVSGIDLSRYAMSWVRQAPGEGLEYIGSISSGGITYYASWAKGRFTISKTSTTVDLKITSPTTEDTGTYFCGRVGTGDSVGIWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPMCPPPELPGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPTVLDSDGSYFLYSKLSVPTSEWQRGDVFTCSVMHEALHNHYTQKSISRSPGK

SEQ ID NO: 8 - protein, 238 aa, synthetic construct
  source 1..238: note = 2G10-L Light Chain
MDTRAPTQLLGLLLLWLPGATFAIVMTQTPSSKSVPVGGTVTINCQASQSVYKNNYLSWYQQKPGQPPKQLIYLASTLASGVPSRFKGSGSGTQFTLTITDVVCDDAATYYCVGWKTSNTDGYGFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 9 - DNA, 1365 bp, synthetic construct
  source 1..1365: mol_type = other DNA; note = 1A4-H Heavy Chain
atggagactgggctgcgctggcttctcctggtcgctgtgctcaaaggtgtccagtgtcagtcgctggaggagtccgggggtcgcctggtcacgcctgggacacccctgacactcacctgcacagtctctggattctccctcagtagctatgctgtgatctgggtccgccaggctccaggggaggggctggaatacatcggaattatttttggtggtggtaagacatactacgcgagctgggcgaaaggccgattcaccatctccaagacctcgaccacggtggatctgagaatcaccagtccgacaaccgaggacacggccacctatttctgtgtcagagatgattggagtagatatttgtggggccaaggcaccctggtcaccgtctcctcagggcaacctaaggctccatcagtcttcccactggccccctgctgcggggacacacccagctccacggtgaccctgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccatgtgcccaccccctgaactcccggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccgaccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatag

SEQ ID NO: 10 - DNA, 720 bp, synthetic construct
  source 1..720: mol_type = other DNA; note = 1A4-L Light Chain
atggacacgagggcccccactcagctgctggggctcctgctgctctggctcccaggtgccacatttgcccaagtgctgacccagactgcatcgcccgtgtctgcagctgtgggaagcacagtcaccatcaattgccagtccagtgagagtgtttatagtaacaactacttatcctggtttcaccagaaaccagggcagcctcccaagcaactgatctattctgcatccactctggcatctggggtcccatcgcggttcaaaggcagtggatctgggacacagttcactctcaccatcagcgacgtgcagtgtgacgatgctgccacttactactgtctgggcaattatggttgtagtagtgctgattgtcgggctttcggcggagggaccgaggtggtggtcaaaggtgatccagttgcacctactgtcctcatcttcccaccagctgctgatcaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcaataggggtgactgttag

SEQ ID NO: 11 - DNA, 1356 bp, synthetic construct
  source 1..1356: mol_type = other DNA; note = 1E10-H Heavy Chain
atggagactgggctgcgctggcttctcctggtcgctgtgctcaaaggtgtccagtgtcagtcagtgaaggagtccgggggtcgcctggtcacgcctgggacacccctgacactcacctgcacagtctctggaatggacctcagtagtaatgtggtgagttgggtccgccaggctccagggaaggggccggaatggatcgtaactattggtggtagtggtaggacattttacgcgacctgggcgaaaggccgattcaccgtctccaaaacctcgacctcggtggatctgaaagtcaccagtctgacaaccgaggacacggccacttatttctgtgccagactttgggacatctggggcccaggcaccctggtcaccgtctcctcagggcaacctaaggctccatcagtcttcccactggccccctgctgcggggacacacccagctccacggtgaccctgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccatgtgcccaccccctgaactcccggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccgaccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatag

SEQ ID NO: 12 - DNA, 711 bp, synthetic construct
  source 1..711: mol_type = other DNA; note = 1E10-L Light Chain
atggacacgagggcccccactcagctgctggggctcctgctgctctggctcccaggtgccacattcgcgcaagtgctgacccagactccatcctccgtgtctgcagctgtgggaggcacagtcaccataagttgccggtccagtcagagtgtttatgcgaacaacgccttatcctggtatcagcagaaaccagggcagcctcccaagctcctgatcgattctgctaccactctgccatctggggtcccatcgcgattcagaggcttcggatctgggacacagttcactctcaccatcagcgacctggagtgtgacgatgctgccacttactactgtgcaggcggttatagtggtgtcatttatgttttcggcggagggaccgaggtggtggtcaaaggtgatccagttgcacctactgtcctcatcttcccaccagctgctgatcaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcaataggggtgactgttag

SEQ ID NO: 13 - DNA, 1368 bp, synthetic construct
  source 1..1368: mol_type = other DNA; note = 2H10-H Heavy Chain
atggagactgggctgcgctggcttctcctggtcgctgtgctcaaaggtgtccagtgtcagtcggtggaggagtccgggggtcgcctggtcacgcctggaggatccctgacactcacctgtaccgtctctggattctccctcagtagctatggaatgaactgggtccgccaggctccaggtgagggactgaaatggatcggaaccattactactggtggtcgcgcatattacgcgagctgggcgaaaggccgattcaccatctccagaacctcgtcgaccacggtgaatctgaaaatgaccagtctgacagccgaggacacggccacctatttttgttacagagctgattacactactgccatctggggcccaggcaccctggtcaccgtctcctcagggcaacctaaggctccatcagtcttcccactggccccctgctgcggggacacacccagctccacggtgaccctgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccatgtgcccaccccctgaactcccggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccgaccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatag

SEQ ID NO: 14 - DNA, 717 bp, synthetic construct
  source 1..717: mol_type = other DNA; note = 2H10-L Light Chain
atggacacgagggcccccactcagctgctggggctcctgctgctctggctcccaggtgccagatgtgcccttgtgatgacccagactccatcccccgtgtctgcacctgtgggaggcacagtcaccatcagttgccaggccagtcagagtgttgctaataacaacgacttagcctggtttcagcagaaaccagggcagcctcccaagctcctgatctatggtgcatccactctggcatctggggtcccatcgcggttcaaaggcagtggatctgggacacagttcactctcaccatcgacggcgtgcagtgtgacgatgctgccagttactattgtgtaggctataaaagttttagtaatgatgattttggtttcggcggagggaccgaggtggtggtcaaaggtgatccagttgcacctactgtcctcatcttcccaccagctgctgatcaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcaataggggtgactgttag

SEQ ID NO: 15 - DNA, 1371 bp, synthetic construct
  source 1..1371: mol_type = other DNA; note = 2G10-H Heavy Chain
atggagactgggctgcgctggcttctcctggtcgctgtgctcaaaggtgtccagtgtcagtcggtggaggagtccgggggtcgcctggtcacgcctgggacacccctgacactcacctgcacagtctctggaatcgacctcagtaggtatgcaatgagctgggtccgccaggctccaggggagggcctggaatacatcggatccattagtagtggtggtatcacatattacgcgagctgggcgaaaggccgattcaccatctccaaaacctcgaccacggtggatctcaaaatcaccagtccgacaaccgaggacacgggcacctatttctgtggcagagtcggaactggtgatagtgttggcatctggggcccaggcaccctggtcaccgtctcctcagggcaacctaaggctccatcagtcttcccactggccccctgctgcggggacacacccagctccacggtgaccctgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccatgtgcccaccccctgaactcccggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccgaccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatag

SEQ ID NO: 16 - DNA, 717 bp, synthetic construct
  source 1..717: mol_type = other DNA; note = 2G10-L Light Chain
atggacacgagggcccccactcagctgctggggctcctgctgctctggctcccaggtgccacatttgccatcgtgatgacccagactccatcttccaaatctgtccctgtgggaggcacagtcaccatcaattgccaggccagtcagagtgtttataagaacaactacttatcctggtatcagcagaaaccagggcagcctcccaagcaactgatctatctggcatccactctggcatctggggtcccatcgcggttcaaaggcagtggatctgggacacagttcactctcaccatcaccgatgtggtgtgtgacgatgctgccacttactactgtgtaggatggaaaactagtaatactgatgggtatggtttcggcggagggaccgaggtggtggtcaaaggtgatccagttgcacctactgtcctcatcttcccaccagctgctgatcaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcaataggggtgactgttag

SEQ ID NO: 17 - protein, 10 aa, synthetic construct
LXXXLXXLXL

SEQ ID NO: 18 - protein, 4 aa, synthetic construct
KDEL

SEQ ID NO: 19 - protein, 15 aa, synthetic construct
GGGGGGGGGGGGGGG

SEQ ID NO: 20 - protein, 15 aa, synthetic construct
GGGGSGGGGSGGGGS